(19) United States
(12) Patent Application Publication
(10) Pub. No.: US 2004/0072839 A1
(43) Pub. Date: Apr. 15, 2004

(54) 1-Phenylalkylpiperazines

(76) Inventors: Amedeo Leonardi, Milan (IT); Gianni Motta, Barlassina (IT); Carlo Riva, Varese (IT); Elena Poggesi, Milano (IT)

Correspondence Address: Mitchell Bernstein, Ph.D., DARBY & DARBY P.C., 805 Third Avenue, New York, NY, 10022, US

(21) Appl. No.: 10/463,196

(22) Filed: Jun. 16, 2003

Related U.S. Application Data
(60) Provisional application No. 60/505,350, filed on Jun. 14, 2002.

(30) Foreign Application Priority Data
Jun. 14, 2002 (IT) MI2002A 001327

(51) Int. Cl. A61K 31/496; A61K 31/495; C07D 241/04

(52) U.S. Cl. 514/252.12; 544/399; 514/252.13; 544/359

(57) ABSTRACT

Described are novel 1-phenylalkylpiperazines having affinity for serotonergic receptors. These compounds and their enantiomers, diastereoisomers, N-piperazine oxides, polymorphs, solvates and pharmaceutically acceptable salts are useful in the treatment of patients with neuromuscular dysfunction of the lower urinary tract and diseases related to 5-HT_{1A} receptor activity. Also described are the preparation of the compounds and the pharmaceutical compositions containing them.

### CROSS REFERENCE TO RELATED APPLICATIONS

The present application claims the benefit of priority under 35 U.S.C. §119(e) of provisional application No. 60/___,___, filed Jun. 14, 2002 (which is a provisional application converted from non-provisional application Ser. No. 10/172,794, filed Jun. 14, 2002), and the benefit of priority under 35 U.S.C. §119(a)-(d) of Italian patent application M12002A 001327, filed Jun. 14, 2002. Each of the foregoing applications is hereby incorporated herein by reference in its entirety.

### FIELD OF THE INVENTION

This invention relates to novel 1-phenylalkylpiperazines having affinity for serotonergic receptors, pharmaceutical compositions thereof and uses for such compounds and compositions.

### BACKGROUND OF THE INVENTION

In mammals, micturition (urination) is a complex process that requires the integrated action of the bladder, its internal and external sphincters, the musculature of the pelvic floor, and neurological control over these muscles at three levels (in the bladder wall or sphincter itself, in the autonomic centres of the spinal cord and in the central nervous system at the level of the pontine micturition centre (PMC) in the brainstem (pons) under the control of the cerebral cortex) (De Groat, Neurobiology of Incontinence, Ciba Foundation Symposium 151:27, 1990). Micturition results from contraction of the detrusor muscle, which consists of interlacing smooth-muscle fibres, under the control of the parasympathetic autonomic system originating from the sacral spinal cord. A simple voiding reflex is triggered by sensory nerves for pain, temperature and distension that run from the bladder to the sacral spinal cord. However, sensory tracts from the bladder reach the PMC too, generating nerve impulses that normally suppress the sacral spinal suppression of cortical inhibition of the reflex arc, and relaxing the muscles of the pelvic floor and external sphincter. Finally, the detrusor muscle contracts and voiding occurs. Abnormalities of lower-urinary tract function, e.g., dysuria, incontinence and enuresis, are common in the general population. Dysuria includes urinary frequency, nocturia and urgency, and may be caused by cystitis (including interstitial cystitis), prostatitis or benign prostatic hyperplasia (BPH) (which affects about 70% of elderly males), or by neurological disorders. Incontinence syndromes include stress incontinence, urgency incontinence, overflow incontinence and mixed incontinence. Enuresis refers to the involuntary passage of urine at night or during sleep.

Previously, treatment of neuromuscular dysfunction of the lower urinary tract involved administration of compounds that act directly on the bladder muscles, such as flavoxate, a spasmolytic drug (Ruffinan, J. Int. Med. Res. 16:317, 1988) which is also active on the PMC (Guarneri et al., Drugs of Today, 30:91, 1994), or anticholinergic compounds such as oxybutynin (Andersson, Drugs 36:477, 1988) and tolterodine (Nilvebrant, Life Sci. 68(22-23): 2549, 2001). The use of α1-adrenergic receptor antagonists for the treatment of BPH is common too, but is based on a different mechanism of action (Lepor, Urology, 42:483, 1993). However, treatments that involve direct inhibition of the pelvic musculature (including the detrusor muscle) may have unwanted side effects, such as incomplete voiding or accommodation paralysis, tachycardia and dry mouth (Andersson, Drugs 35:477, 1988). Thus, it would be preferable to utilize compounds that act via the central nervous system to affect for, example, the sacral spinal reflex and/or the PMC inhibition pathways in a manner that restores normal functioning of the micturition mechanism.

U.S. Pat. No. 5,346,896 discloses 5-HT_{1A} binding agents which may be used in the treatment of CNS disorders, such as, for example, anxiety.

U.S. Pat. Nos. 6,239,135; 6,358,958 and 6,514,976 disclose aryl piperazine compounds that bind to 5-HT_{1A} receptors.

International publication number WO 94/15919 discloses piperazine derivatives that are 5-HT_{1A} binding agents.

### SUMMARY OF THE INVENTION

The present invention provides compounds of formula I:

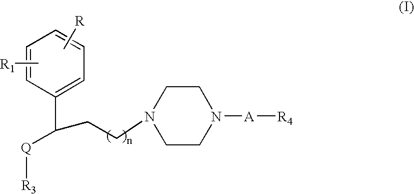

wherein

R represents hydrogen or one or more substituents selected from the group consisting of (C₁-C₆)-alkyl, (C₁-C₆)-alkoxy, (C₁-C₆)-alkylthio, hydroxy, halo, (C₂-C₆)-alkenyl, (C₂-C₆)-alkynyl, (C₁-C₆)-haloalkyl, (C₁-C₆)-haloalkoxy, (C₁-C₆)-hydroxyalkyl, alkoxyalkyl, nitro, amino, (C₁-C₆)-aminoalkyl, (C₁-C₆)-alkylamino-(C₁-C₆)-alkyl, (C₁-C₆)-alkylamino, di-(C₁-C₆)-alkylamino, acylamino, (C₁-C₆)-alkylsulphonylamino, aminosuiphonyl, (C₁-C₆)-alkylaminosuiphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups;

R₁ represents a member selected from the group consisting of hydrogen, cycloalkyl, aryl, aryloxy, aralkyl, aralkoxy, heterocyclic, heterocycloxy, heterocycloalkyl and heterocycloalkoxy groups, each group being optionally substituted with one or more substituent R, defined as above;

Q represents —C(O)— or —CH(OR₂)— where R₂ represents a member selected from the group consisting of hydrogen, (C₁-C₆)-alkyl, (C₂-C₆)-alkenyl, (C₂-C₆)-alkynyl and cycloalkyl groups, wherein each group is optionally substituted with one or more groups selected from R₅ and R₆, where R₅ is selected from the group consisting of halo, (C₁-C₆)-alkoxy, (C₁-C₆)-haloalkoxy, cyano, (C₁-C₆)-alkoxycarbonyl, (C₁-C₆)-alkylcarbonyl, alkoxyalkyl, aminocarbonyl, N-(C₁-C₆)-alkylaminocarbonyl, N,N-di-(C₁-C₆)-alkylaminocarbonyl groups and R₆ is selected from the group consisting of aryl, heteroaryl, aryloxy, heteroaryloxy, arylalkoxy, and heteroarylalkoxy groups, each optionally substituted with R, or R₂ represents —C(O)—(C₁-C₆)-alkyl, —C(O)O—(C₁-C₆)-alkyl, —C(O)NR₇R₈ or —C(S)NR₇R₈ wherein R₇ and R₈ are independently hydrogen or (C₁-C₆)-alkyl;

R₃ represents hydrogen or a (C₁-C₆)-alkyl, (C₂-C₆)-alkenyl, (C₂-C₆)-alkynyl, cycloalkyl, aryl or heterocycle group, each group being optionally substituted with one or more substituent R or R₁, defined as above;

R₄ represents an aryl or heterocyclic group, each being optionally substituted with one or more substituent R, defined as above;

A represents a bond or (CH₂)ₙ; and

n=1 or 2,

or an enantiomer, optical isomer, diastereomer, N-oxide (e.g., N-piperazine oxide), crystalline form, hydrate, solvate or pharmaceutically acceptable salt thereof.

As referred to in the definition of R₆, aryl, heteroaryl, aryloxy, heteroaryloxy, arylalkoxy and heteroarylalkoxy group may be optionally substituted with one or more substituents selected from the group consisting of, (C₁-C₆)-alkyl, (C₁-C₆)-alkoxy, (C₁-C₆)-alkylthio, hydroxy, halo, (C₂-C₆)-alkenyl, (C₂-C₆)-alkynyl, (C₁-C₆)-haloalkyl, (C₁-C₆)-haloalkoxy, (C₁-C₆)-hydroxyalkyl, alkoxyalkyl, nitro, amino, (C₁-C₆)aminoalkyl, (C₁-C₆)-alkylamino(C₁-C₆)-alkyl, (C₁-C₆)-alkylamino, di(C₁-C₆)-alkylamino, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, formyl, alkylcarbonylalkyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups.

In certain preferred embodiments, Q represents —CH(OR₂)—, where R₂ is defined as above.

In a preferred embodiment, the invention provides compounds of formula I as defined above, with the proviso that the substituents of formula I are not such that simultaneously R=hydrogen or (C₁-C₆)-alkyl, R₁=halogen, Q=—C(O)— or —CH(OR₂)— where R₂=hydrogen, R₃=cycloalkyl or alkyl and R₄=phenyl substituted with a member selected from the group consisting of (C₁-C₆)-alkyl, (C₁-C₆)-alkoxy and (C₁-C₆)-haloalkoxy groups, A is a bond and n=1.

In another preferred embodiment, the invention provides compounds of formula I as defined above, with the proviso that the substituents of formula I are not such that simultaneously Q=—CH(OR₂) where R₂=H; R₃=cycloalkyl; R=2-fluoro; R₁=H, R₄=2-methoxyphenyl or 2-(2,2,2-trifluoroethoxy)phenyl; A=bond; and n=1.

Also preferred is an embodiment wherein the invention provides compounds of formula I as defined above with the proviso that the substituents of formula I are not such that simultaneously Q=—C(O)— or —CH(OR₂)— where R₂=hydrogen; R₁=H, phenyl or phenyl substituted with halo, (C₁-C₆)-alkyl or (C₁-C₆)-alkoxy; R=H, (C₁-C₆)-alkyl, (C₁-C₆)-alkoxy, halo, haloalkyl, nitro, amino, (C₁-C₆)-alkylamino or di-(C₁-C₆)-alkylamino; R₄ is an unsubstituted aryl, unsubstituted heteroaryl or an aryl or heteroaryl group substituted with one or more substituent selected from the group consisting of (C₁-C₆)-alkyl, (C₁-C₆)-alkoxy, halo, (C₁-C₆)-haloalkyl, nitro, amino, (C₁-C₆)-alkylamino, di-(C₁-C₆)-alkylamino, hydroxy, (C₁-C₆)-hydroxyalkyl, —CONR₇R₈, wherein R₇ and R₈ are independently hydrogen or (C₁-C₆)-alkyl, and —NHSO₂—(C₁-C₆)-alkyl groups; A is a bond; and R₃ represents unsubstituted aryl, unsubstituted heteroaryl, or aryl or heteroaryl substituted with one more substituent selected from group consisting of (C₁-C₆)-alkyl, (C₁-C₆)-alkoxy, halo, (C₁-C₆)-haloalkyl, nitro, amino, (C₁-C₆)-alkylamino, di-(C₁-C₆)-alkylamino, phenyl, halophenyl, (C₁-C₆)-alkylphenyl and (C₁-C₆)-alkoxyphenyl groups.

Also preferred is an embodiment wherein the invention provides compounds of formula I as defined above with the proviso that the substituents of formula I are not such that simultaneously Q=—C(O)— or —CH(OR₂)— where R₂=hydrogen; R₁=H or unsubstituted cycloalkyl or cycloalkyl substituted with (C₁-C₆)-alkyl; R=H, (C₁-C₆)-alkyl, (C₁-C₆)-alkoxy, halo, (C₁-C₆)-haloalkyl, (C₁-C₆)-alklythio, (C₂-C₆)-alkenyl or (C₂-C₆)-alkynyl; R₄ is an unsubstituted aryl, unsubstituted heteroaryl, or an aryl or heteroaryl substituted with one to three substituents selected from the group consisting of (C₁-C₆)-alkyl, (C₁-C₆)-alkoxy, halo, (C₁-C₆)-haloalkyl, (C₁-C₆)-alklythio, (C₂-C₆)-alkenyl and (C₂-C₆)-alkynyl groups; A is a bond; and R₃ represents unsubstituted phenyl, unsubstituted naphthyl or unsubstituted cycloalkyl, or phenyl, naphthyl or cycloalkyl substituted with one to three substituents selected from the group consisting of (C₁-C₆)-alkyl, (C₁-C₆)-alkoxy, halo, (C₁-C₆)-haloalkyl, (C₁-C₆)-alklythio, (C₂-C₆)-alkenyl and (C₂-C₆)-alkynyl groups.

Also preferred is an embodiment wherein the invention provides compounds of formula I as defined above with the proviso that the substituents of formula I are not such that simultaneously Q represents —C(O)—; R represents one more substituents selected from the group consisting of (C₁-C₆)-alkyl, (C₁-C₆)-alkoxy, (C₁-C₆)-alkylthio, halo, (C₁-C₆)-polyhaloalkyl, nitro, amino, N-(C₁-C₆)-alkylamino, N,N-di-(C₁-C₆)-alkylamino and cyano groups; R₁ represents a hydrogen atom; R₄ is a radical selected from the group consisting of indolyl, isoindolyl, quinolinyl, isoquinolinlyl, indazolyl and benzotriazolyl, or one of the foregoing radicals substituted with one or more substituent selected from the group consisting of (C₁-C₆)-alkyl, (C₁-C₆)-alkoxy, hydroxy, halo, (C₁-C₆)-haloalkyl, (C₁-C₆)-hydroxyalkyl, alkoxyalkyl, nitro, amino, N-(C₁-C₆)-alkylamino, N,N-di-(C₁-C₆)-alkylamino, (C₁-C₆)-alkoxycarbonyl, (C₁-C₆)-alkylcarbonyl, alkylcarbonylalkyl and alkanoyloxyalkyl groups; and R₃ represents a saturated heterocyclic ring comprising a nitrogen atom, through which said saturated heterocyclic ring is bonded to the adjacent carbonyl group at Q, and which may optionally include a further hetero atom, and which may also be optionally substituted with an alkyl group.

In each of the preferred embodiments, it is further preferred that Q is —CH(OR₂)—.

Compounds of formula I can exist as four stereoisomers, which may be present in racemic mixtures or in any other combination. Racemic mixtures can be subjected to enantiomeric enrichment, to yield compositions enriched in a particular enantiomer, which can be incorporated into compositions comprising a single enantiomer. Enantiomeric enrichment can be expressed as ee (enantiomeric excess) as defined below.

Particular specific embodiments within the formula I include:

1-[4-cyclohexyl-3-(2-fluorophenyl)-4-methoxybutyl]-4-[2-(2,2,2-trifluoroethoxy)phenyl]piperazine;

1-(4-Fluoro-2-methoxyphenyl)-4-[4-oxo-3-(2-trifluoromethoxyphenyl)pentyl]piperazine;

1-(4-Fluoro-2-methoxyphenyl)-4-[4-hydroxy-3-(2-trifluoromethoxyphenyl)pentyl]piperazine;

1-[5-(2,3-Dihydro-1,4-benzodioxinyl)]-4-[4-oxo-3-(2-trifluoromethoxyphenyl)pentyl]piperazine;

1-[5-(2,3-Dihydro-1,4-benzodioxinyl)]-4-[4-hydroxy-3-(2-trifluoromethoxyphenyl)pentyl]piperazine;

1-[5-(2,3-Dihydro-1,4-benzodioxinyl)]-4-[4-hydroxy-3-(2-trifluoromethoxyphenyl)hexyl]piperazine;

1-[5-(2,3-Dihydro-1,4-benzodioxinyl)]-4-[4-hydroxy-3-(2-trifluoromethoxyphenyl)hex-5-enyl]piperazine;

1-[5-(2,3-Dihydro-1,4-benzodioxinyl)]-4-[4-hydroxy-5-methyl-3-(2-trifluoromethoxyphenyl)hexyl]piperazine;

1-[5-(2,3-Dihydro-1,4-benzodioxinyl)]-4-[4-methoxy-3-(2-trifluoromethoxyphenyl)-5-hexenyl]piperazine;

1-[5-(2,3-Dihydro-1,4-benzodioxinyl)]-4-[(4-methoxy-3-phenyl)heptyl]piperazine;

1-[5-(2,3-Dihydro-1,4-benzodioxinyl)]-4-[(4-methoxy-3-phenyl)pentyl]piperazine;

1-[5-(2,3-Dihydro-1,4-benzodioxinyl)]-4-[(4-propoxy-3-phenyl)heptyl]piperazine;

1-[3-(2-Cyanophenyl)-4-cyclohexyl-4-oxo-butyl]-4-[5-(2,3-dihydro-1,4-benzodioxinyl)]piperazine;

1-[3-(2-Cyanophenyl)-4-cyclohexyl-4-hydroxybutyl]-4-[5-(2,3-dihydro-1,4-benzodioxinyl)]piperazine;

1-[3-(2-Cyanophenyl)-4-cyclohexyl-4-hydroxybutyl]-4-[5-(2,3-dihydro-1,4-benzodioxinyl)]piperazine;

1-[3-(2-Cyanophenyl)-4-cyclohexyl-4-hydroxybutyl]-4-[5-(2,3-dihydro-1,4-benzodioxinyl)]piperazine;

1-[3-(2-Cyanophenyl)-4-cyclohexyl-4-oxobutyl]-4-(4-fluoro-2-methoxyphenyl)piperazine;

1-[3-(2-Cyanophenyl)-4-cyclohexyl-4-hydroxybutyl]-4-(4-fluoro-2-methoxyphenyl)piperazine;

1-(4-cyclohexyl-4-methoxy-3-phenylbutyl)-4-[5-(2,3-dihydro-1,4-benzodioxinyl)]piperazine;

1-(4-Cyclohexyl-4-methoxy-3-phenylbutyl)-4-(4-fluoro-2-methoxyphenyl)piperazine;

1-(4-Cyclohexyl-4-ethoxy-3-phenylbutyl)-4-[5-(2,3-dihydro-1,4-benzodioxinyl)]piperazine;

1-(4-Cyclohexyl-4-ethoxy-3-phenylbutyl)-4-(4-fluoro-2-methoxyphenyl)piperazine;

1-(4-Allyloxy-4-cyclohexyl-3-phenylbutyl)-4-[5-(2,3-dihydro-1,4-benzodioxinyl)]piperazine;

1-(4-Allyloxy-4-cyclohexyl-3-phenylbutyl)-4-(4-fluoro-2-methoxyphenyl)piperazine;

1-(4-Cyclohexyl-3-phenyl-4-propargyloxybutyl)-4-[5-(2,3-dihydro-1,4-benzodioxinyl)]piperazine;

1-(4-Cyclohexyl-3-phenyl-4-propargyloxybutyl)-4-(4-fluoro-2-methoxyphenyl)piperazine;

1-(4-Cyclohexyl-3-phenyl-4-propoxybutyl)-4-[5-(2,3-dihydro-1,4-benzodioxinyl)]piperazine;

1-[5-(2,3-Dihydro-1,4-benzodioxinyl)]-4-(4-hydroxy-3-phenyl)hexylpiperazine;

1-[5-(2,3-Dihydro-1,4-benzodioxinyl)]-4-(4-hydroxy-3-phenyl)heptyl]piperazine;

1-[5-(2,3-Dihydro-1,4-benzodioxinyl)]-4-(4-hydroxy-3-phenylhex-5-enyl]piperazine;

1-[5-(2,3-Dihydro-1,4-benzodioxinyl)]-4-(4-hydroxy-5-methyl-3-phenyl)hexyl]piperazine;

1-[5-(2,3-Dihydro-1,4-benzodioxinyl)]-4-(4-hydroxy-3-phenyl)pentyl]piperazine;

1-[5-(2,3-Dihydro-1,4-benzodioxinyl)]-4-(4-hydroxy-3-phenylhept-5-ynyl)piperazine;

1-[5-(2,3-Dihydro-1,4-benzodioxinyl)]-4-(4-hydroxy-3-phenylhept-5-enyl)piperazine;

1-[5-(2,3-Dihydro-1,4-benzodioxinyl)]-4-(4-hydroxy-3-phenylhex-5-ynyl)piperazine;

1-[5-(2,3-Dihydro-1,4-benzodioxinyl)]-4-(4-hydroxy-3-phenylhept-6-enyl)piperazine;

1-[5-(2,3-Dihydro-1,4-benzodioxinyl)]-4-(4-hydroxy-6-methyl-3-phenylhept-5-enyl)piperazine;

1-[5-(2,3-Dihydro-1,4-benzodioxinyl)]-4-(4-hydroxy-6-methyl-3-phenyl)heptyl]piperazine;

1-[5-(2,3-dihydro-1,4-benzodioxinyl)]-4-(4-hydroxy-3-phenylbutyl]piperazine;

1-[5-(2,3-Dihydro-1,4-benzodioxinyl)]-4-(4-hydroxy-3-phenylpentyl)piperazine;

1-[4-Cyclohexyl-3-(2-dimethylaminocarbonylphenyl)-4-oxobutyl]-4-[5-(2,3-dihydro-1,4-benzodioxinyl)]piperazine;

1-[4-Cyclohexyl-3-(2-dimethylaminocarbonylphenyl)-4-hydroxybutyl]-4-[5-(2,3-dihydro-1,4-benzodioxinyl)]piperazine;

1-[4-Cyclohexyl-3-(2-dimethylaminocarbonylphenyl)-4-oxobutyl]-4-(4-fluoro-2-methoxyphenyl)piperazine;

1-[4-Cyclohexyl-3-(2-dimethylaminocarbonylphenyl)-4-hydroxybutyl]-4-(4-fluoro-2-methoxyphenyl)piperzine;

1-[3-(2-Cyanophenyl)-4-oxopentyl]-4-[5-(2,3-dihydro-1,4-benzodioxinyl)]piperazine;

1-[4-Cyclohexyl-3-(2-trifluoromethoxyphenyl)-4-oxobutyl]-4-(4-indolyl)piperazine;

1-[4-Acetoxy-4-cyclohexyl-3-(2-fluorophenyl)butyl]-4-(2-methoxyphenyl)piperazine;

1-[4-Cyclohexyl-3-(2-fluorophenyl)-4-methoxycarbonyloxybutyl]-4-(2-methoxyphenyl)piperazine;

1-[4-Cyclohexyl-4-ethylaminocarbonyloxy-3-(2-fluorophenyl)butyl]-4-(2-methoxyphenyl)piperazine;

1-[4-Aminocarbonyloxy-4-cyclohexyl-3-(2-fluorophenyl)butyl]-4-(2-methoxyphenyl)piperazine;

1-[5-(2,3-Dihydro-1,4-benzodioxinyl)]-4-(4-hydroxy-5,5-dimethyl-3-phenyl)hexyl]piperazine;

1-(4-Fluoro-2-methoxyphenyl)-4-[(4-hydroxy-3-phenyl)hept-5-ynyl]piperazine;

1-(4-Fluoro-2-methoxyphenyl)-4-[(4-hydroxy-3-phenyl)hept-5-enyl]piperazine;

1-(4-Fluoro-2-methoxyphenyl)-4-[(4-hydroxy-3-phenyl)hept-5-enyl]piperazine;

1-[5-(2,3-Dihydro-1,4-benzodioxinyl)]-4-[(4-hydroxy-5-methyl-3-phenyl)hex-5-enyl]piperazine;

1-[5-(2,3-Dihydro-1,4-benzodioxinyl)]-4-[(4-hydroxy-6-methyl-3-phenyl)hept-6-enyl]piperazine;

1-[5-(2,3-Dihydro-1,4-benzodioxinyl)]-4-[[4-hydroxy-4-(2-thienyl)-3-phenyl]butyl]piperazine;

1-[5-(2,3-Dihydro-1,4-benzodioxinyl)]-4-[(4-hydroxy-3-phenyl)octyl]piperazine;

1-(4-Fluoro-2-methoxyphenyl)-4-[(4-methoxy-3-phenyl)hept-5-ynyl]piperazine;

1-(4-Fluoro-2-methoxyphenyl)-4-[(4-methoxy-3-phenyl)hept-5-enyl]piperazine;

1-[4-Cyclohexyl-3-(2-methoxymethylphenyl)-4-oxobutyl]-4-(4-fluoro-2-methoxyphenyl)piperazine;

1-[4-Cyclohexyl-4-hydroxy-3-(2-methoxymethylphenyl)-butyl]-4-(4-fluoro-2-methoxyphenyl)piperazine;

1-[5-(2,3-Dihydro-1,4-benzodioxinyl)]-4-[4-cyclohexyl-3-(2-methoxymethylphenyl)-4-oxobutyl]-piperazine;

1-[4-Cyclohexyl-4-hydroxy-3-(2-methoxymethylphenyl)-butyl]-4-(2,3-dihydro-1,4-benzodioxinyl)piperazine; and

1-[4-Cyclohexyl-4-methylaminothiocarbonyloxy-3-(2-fluorophenyl)butyl]-4-(2-methoxyphenyl)piperazine.

In certain embodiments, the invention provides isomers of the aforementioned compounds of formula I, as, for example, a pure enantiomer or, alternatively, a mixture of any two or more enantiomers in any proportion. Preferably, enantiomers of compounds of formula I are provided in predetermined amounts.

The invention also includes metabolites of the foregoing compounds having the same type of activity, hereinafter referred to as active metabolites.

In certain preferred embodiments, the invention provides the foregoing compounds that are antagonists of serotonergic 5-HT_{1A} receptors.

The present invention also contemplates prodrugs which are metabolized in the body to generate any of the foregoing compounds.

In another embodiment, the present invention provides pharmaceutical compositions comprising the foregoing compounds, enantiomers, diastereomers, N-piperazine oxides, crystalline forms, hydrates, solvates or pharmaceutically acceptable salts of such compounds of formula I, in admixture with pharmaceutically acceptable diluents or carriers such as those disclosed.

In another embodiment, the invention provides intermediates useful in the synthesis of compounds of formula I.

Yet another embodiment is a method for reducing the frequency of bladder contractions due to bladder distension in a mammal (such as a human) in need thereof by administering an effective amount of at least one compound of the present invention having affinity for serotonergic 5-HT_{1A} receptors (and preferably antagonist activity) reduce the frequency of bladder contractions due to bladder distension to the mammal.

Yet another embodiment is a method for increasing urinary bladder capacity in a mammal (such as a human) in need thereof by administering an effective amount of at least one compound of the present invention to increase urinary bladder capacity to the mammal.

Yet another embodiment is a method for treating disorders of the urinary tract in a mammal (such as a human) in need thereof by administering an effective amount of at least one compound of the present invention to ameliorate at least one condition among urinary urgency, overactive bladder, increased urinary frequency, decreased urinary compliance (decreased bladder storage capacity), cystitis (including interstitial cystitis), incontinence, urine leakage, enuresis, dysuria, urinary hesitancy and difficulty in emptying the bladder.

In yet other embodiments, the invention provides a method for treating a mammal suffering from a central nervous system (CNS) disorder due to serotonergic dysfunction by administering an effective amount of at least one compound of the present invention to treat the CNS disorder. Such dysfunctions include, but are not limited to, anxiety, depression, hypertension, sleep/wake cycle disorders, feeding, behaviour, sexual dysfunction and cognition disorders in mammals (particularly in humans) associated with stroke, injury, dementia, and originated by neurological development, attention-deficit hyperactivity disorders (ADHD), drug addiction, drug withdrawal, irritable-bowel syndrome and symptoms caused by withdrawal or partial withdrawal from the use of nicotine or tobacco.

In yet another embodiment, the invention provides a method for treating a disorder due to serotonergic dysfunction by delivering a compound of the invention to the environment of a 5-HT_{1A} serotonergic receptor, for example, to the extracellular medium (or by systemically or locally administering to a mammal possessing such a 5-HT_{1A} receptor) an amount of a compound of the invention effective in the treatment of said disorder due to serotonergic dysfunction.

In a preferred embodiment, the invention provides methods for treating a mammal (including a human) suffering from a urinary tract disorder by administering at least one compound of the invention to the environment of a 5-HT_{1A} receptor in an amount effective to increase the duration of bladder quiescence. More highly preferred are compounds and/or amounts administered which accomplish an increase in the duration of bladder quiescence is with little or no effect (e.g., decrease or increase) on micturition pressure.

In yet other embodiments, the invention provides for methods of treating the above disorders, by administering a compound of formula I in combination with other agents such as, for example, one or more additional 5-HT_{1A} antagonist, antimuscarinic drugs, α1-adrenergic antagonists, inhibitors of the cyclooxygenase enzyme, which may inhibit both COX1 and COX2 isozymes or which may, alternatively, be selective for COX2 isozyme, and NO donor derivatives thereof.

### DETAILED DESCRIPTION OF THE INVENTION

All patents, patent applications and literature references cited in the description are hereby incorporated herein by reference in their entireties. In the case of inconsistencies, the present disclosure, including definitions, will prevail.

### Compounds

The present invention is related to compounds of formula I as disclosed above. The invention includes the enantiomers, diastereoisomers, N-piperazine oxides, crystalline forms, hydrates, solvates or pharmaceutically acceptable salts of these compounds, as well as active metabolites of these compounds having the same type of activity.

The term “alkyl” refers, either alone or within other terms such as, for example and without limitation, “haloalkyl” and “alkylsulfonyl”, to saturated linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” refers to linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of such radicals include ethenyl, n-propenyl, butenyl, and the like.

The term “alkynyl” refers to linear or branched radicals having at least one carbon-carbon triple bond of two to about twenty carbon atoms or, preferably, two to about twelve carbon atoms. More preferred alkynyl radicals are “lower alkynyl” radicals having two to about six carbon atoms. Examples of such radicals include, n-propynyl, butynyl, and the like.

The terms “halo” and “halogen” are synonymous and refer to halogen atoms such as fluorine, chlorine, bromine or iodine atoms.

The term “haloalkyl” refers to radicals wherein any one or more of the alkyl carbon atoms are substituted independently with one or more halogen atoms. Specifically included are monohaloalkyl and polyhaloalkyl radicals. A monohaloalkyl radical, for one example, may have either an iodo, bromo, chloro or fluoro atom within the radical. Polyhaloalkyl radicals may have two or more of the same halo atoms or a combination of different halo atoms. Each carbon atom within a polyhaloalkyl radical may be substituted independently with one, two, or three halogen atoms, which may be the same or different, with respect to both the halogen atoms on a single carbon atom and the halogen atoms between or among different carbon atoms. Preferred haloalkyl radicals are “lower haloalkyl” radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” refers to linear or branched alkyl radicals having one to about ten carbon atoms any one of which may be substituted with one or more hydroxyl radicals. Preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “alkoxy” refers to linear or branched oxy-containing radicals each having alkyl portions of one to about ten carbon atoms, such as methoxy radical. Preferred alkoxy radicals are “lower alkoxy” radicals having one to six carbon atoms. Examples of such radicals include methoxy, ethoxy, propoxy, butoxy and tert-butoxy.

The term “alkoxyalkyl” refers to alkyl radicals having one or more alkoxy radicals attached to an alkyl radical, that is, to form, e.g., monoalkoxyalkyl or dialkoxyalkyl radicals. Preferred alkoxyalkyl radicals are “lower alkoxyalkyl” radicals having one to six carbon atoms and one or two alkoxy radicals. Examples of such radicals include methoxymethyl, methoxyethyl, ethoxyethyl, methoxybutyl and methoxypropyl.

The term “haloalkoxy” refers to “alkoxy” radicals further substituted with one (i.e., “monohaloalkoxy”) or more than one (i.e., “polyhaloalkoxy”) halo atoms, such as iodo, fluoro, chloro, or bromo. Examples of such radicals include fluoromethoxy, chloromethoxy, trifluoromethoxy, trifluoroethoxy, fluoroethoxy and fluoropropoxy.

The term “aryl”, alone or in combination, refers to a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl.

The terms “heterocyclic” and “heterocyclo” refer to saturated, partially saturated and unsaturated heteroatom-containing ring-shaped radicals, where the heteroatoms may be selected from nitrogen, sulfur and oxygen. Examples of saturated heterocyclic radicals include saturated heteromonocylic groups containing 1 to 4 nitrogen atoms (e.g., pyrrolidinyl, imidazolidinyl, piperidino, piperazinyl); saturated heteromonocyclic groups containing 1 to 2 oxygen atoms and 1 to 3 nitrogen atoms (e.g., morpholinyl); saturated heteromonocyclic groups containing 1 to 2 sulfur atoms and 1 to 3 nitrogen atoms (e.g., thiazolidinyl). Examples of partially saturated heterocyclic radicals include dihydrothiophene, dihydropyran, dihydrofuran and dihydrothiazole.

The terms “heterocyclo” and “heterocyclic” encompass the term “heteroaryl,” which refers to unsaturated heterocyclic radicals. Examples of “heteroaryl” radicals include unsaturated 5 to 6 membered heteromonocyclic groups containing 1 to 4 nitrogen atoms, for example, pyrrolyl, pyrrolinyl, imidazolyl, pyrazolyl, 2-pyridyl, 3-pyridyl, 4-pyridyl, pyrimidyl, pyrazinyl, pyridazinyl, triazolyl (e.g., 4H-1,2,4-triazolyl, 1H-1,2,3-triazolyl, 2H-1,2,3-triazolyl) tetrazolyl (e.g., 1H-tetrazolyl, 2H-tetrazolyl); unsaturated condensed heterocyclic groups containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl); unsaturated 3 to 6-membered heteromonocyclic groups containing an oxygen atom, for example, pyranyl, 2-furyl, 3-furyl; unsaturated 5 to 6-membered heteromonocyclic groups containing a sulfur atom, for example, 2-thienyl, 3-thienyl; unsaturated 5- to 6-membered heteromonocyclic groups containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl); unsaturated condensed heterocyclic groups containing 1 to 2 oxygen atoms and 1 to 3 nitrogen atoms (e.g., benzoxazolyl, benzoxadiazolyl); unsaturated 5 to 6-membered heteromonocyclic groups containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl); unsaturated condensed heterocyclic groups containing 1 to 2 sulfur atoms and 1 to 3 nitrogen atoms (e.g., benzothiazolyl, benzothiadiazolyl) and the like. The term “heteroaryl” also refers to radicals where heterocyclic radicals are fused with aryl radicals. Examples of such fused bicyclic radicals include benzofuran, benzothiophene, and the like. Said “heterocyclic group” may have 1 to 3 substituents such as, for example and without limitation, lower alkyl, hydroxy, oxo, amino and lower alkylamino. Preferred heterocyclic radicals include five to ten membered fused or unfused radicals. Examples of heteroaryl radicals include benzofuryl, 2,3-dihydrobenzofuryl, benzothienyl, indolyl, dihydroindolyl, chromanyl, benzopyran, thiochromanyl, benzothiopyran, benzodioxolyl, benzodioxanyl, pyridyl, thienyl, thiazolyl, oxazolyl, furyl, and pyrazinyl.

The term “sulfonyl”, whether used alone or linked to other terms such as alkylsulfonyl, denotes divalent radicals —SO₂—. “Alkylsulfonyl” refers to alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The term “aminosulfonyl” denotes a sulfonyl radical substituted with an amine radical, forming a sulfonamide (—SO₂NH₂). The terms “N-alkylaminosulfonyl” and “N,N-dialkylaminosulfonyl” denote aminosulfonyl radicals substituted on the amino group, respectively, with one alkyl radical, or two alkyl radicals. Aminosulfonyl radicals may also be substituted on the amine group with, e.g., an aryl group. More preferred alkylaminosulfonyl radicals are “lower alkylaminosulfonyl” radicals having one to six carbon atoms. Examples of such lower alkylaminosulfonyl radicals include N-methylaminosulfonyl, N-ethylaminosulfonyl and N-methyl-N-ethylaminosulfonyl.

The term “alkanoyl” refers to radicals having a carbonyl radical as defined below, attached to an alkyl radical. Preferred alkanoyl radicals are “lower alkanoyl” radicals having 1-6 carbon atoms. The alkanoyl radicals may be substituted or unsubstituted, such as formyl, acetyl, propionyl (propanoyl), butanoyl (butyryl), isobutanoyl (isobutyryl), valeryl (pentanoyl), isovaleryl, pivaloyl, hexanoyl or the like.

The term “carbonyl”, whether used alone or with other terms, such as “alkylcarbonyl”, denotes —(C═O)—. The term “alkylcarbonyl” refers to radicals having a carbonyl radical substituted with an alkyl radical. More preferred alkylcarbonyl radicals are “lower alkylcarbonyl” radicals having one to six carbon atoms. Examples of such radicals include methylcarbonyl and ethylcarbonyl. There is an overlap between the terms “alkanoyl” and “alkylcarbonyl”.

The term “alkylcarbonylalkyl”, denotes an alkyl radical substituted with an “alkylcarbonyl” radical.

The term “alkoxycarbonyl” refers to a radical containing an alkoxy radical, as defined above, attached via an oxygen atom to a carbonyl radical, i.e., an ester radical, —C(O)O-Alk. Preferred alkoxycarbonyl radicals are “lower alkoxycarbonyl” radicals having alkoxy radicals of one to six carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl.

The term “alkanoyloxy” refers to an “alkanoyl” radical as defined above linked to an oxygen radical, to generate an ester radical.

The term “alkanoyloxylalkyl” refers to radicals having “alkanoyloxy”, as defined above substituted to an alkyl radical. Preferred alkanoyloxyalkyl radicals are “lower alkanoyloxyalkyl” radicals having lower alkanoyloxy radicals as defined above attached to alkyl radicals of one to six carbon atoms. Examples of such lower alkanoyloxyalkyl radicals include acetoxymethyl, formyloxyethyl, acetyloxyethyl.

The term “aminocarbonyl” when used by itself or with other terms such as “aminocarbonylalkyl”, “N-alkylaminocarbonyl”, “N,N-dialkylaminocarbonyl”, “N-alkyl-N-arylaminocarbonyl”, “N-alkyl-N-hydroxyaminocarbonyl” and “N-alkyl-N-hydroxyaminocarbonylalkyl”, denotes an amide group of the formula —C(═O)NH₂. The terms “N-alkylaminocarbonyl” and “N,N-dialkylaminocarbonyl” denote aminocarbonyl radicals in which the amino groups have been substituted with one alkyl radical and two alkyl radicals, respectively. Preferred are “lower alkylaminocarbonyl” having lower alkyl radicals as described above attached to an aminocarbonyl radical

The term “cycloalkyl” refers to saturated carbocyclic radicals having three to ten carbon atoms. Preferred cycloalkyl radicals are “lower cycloalkyl” radicals having three to seven carbon atoms. Examples include radicals such as cyclopropyl, cyclobutyl, cyclopentyl, cyclohexyl and cycloheptyl. A most preferred cycloalkyl group is cyclohexyl.

The term “alkylthio” refers to radicals containing a linear or branched alkyl radical, of one to ten carbon atoms, attached to a divalent sulfur atom. An example of “alkylthio” is methylthio, (CH₃—S—).

The term “alkylsulfinyl” refers to radicals containing a linear or branched alkyl radical, of one to ten carbon atoms, attached to a divalent —S(═O)— radical.

The term “amino” refers to the radical —NH₂.

The term “aminoalkyl” refers to alkyl radicals substituted with amino radicals. More preferred aminoalkyl radicals are “lower aminoalkyl” having one to six carbon atoms. Examples include aminomethyl, aminoethyl and aminobutyl.

The term “alkylaminoalkyl” refers to aminoalkyl radicals having the nitrogen atom substituted with at least one alkyl radical. More preferred alkylaminoalkyl radicals are “lower alkylaminoalkyl” having one or two one to six carbon atoms radicals attached to a lower aminoalkyl radical as described above.

The terms “N-alkylamino” and “N,N-dialkylamino” denote amino groups which have been substituted with one alkyl radical and with two alkyl radicals, respectively. More preferred alkylamino radicals are “lower alkylamino” radicals having one or two alkyl radicals of one to six carbon atoms, attached to the nitrogen atom. Examples of “alkylamino” include N-methylamino, N-ethylamino, N,N-dimethylamino, N,N-diethylamino or the like.

The term “acyl”, whether used alone, or within a term such as “acylamino”, denotes a radical provided by the residue after removal of hydroxyl from an organic acid. The term “acylamino” refers an amino radical substituted with an acyl group. An examples of an “acylamino” radical is acetylamino or acetamido (CH₃C(═O)—NH—) where the amine may be further substituted with alkyl, aryl or aralkyl.

The term “aralkyl” refers to aryl-substituted alkyl radicals. Preferable aralkyl radicals are “lower aralkyl” radicals having aryl radicals attached to alkyl radicals having one to six carbon atoms. Examples of such radicals include benzyl, diphenylmethyl, triphenylmethyl, phenylethyl and diphenylethyl. The aryl in said aralkyl may be additionally substituted with halo, alkyl, alkoxy, halkoalkyl and haloalkoxy. The terms benzyl and phenylmethyl are interchangeable.

The term “aryloxy” refers to the radical —O-aryl. Examples of such radicals include phenoxy.

The term “aralalkoxy” refers to the radical -alkoxy-aryl (i.e., —O-alkyl-aryl). Preferred aralkoxy radicals are “lower aralkoxy” radicals having phenyl radicals attached to a lower alkoxy radical as described above. An example of such radical includes benzyloxy.

The term “cyano” refers to the radical —C≡N.

The term “nitro” refers to the radical —NO₂.

The term “heterocycloalkyl” refers to the radical -Alkyl-Heterocycle.

The term “heterocycloxy” refers to the radical —O-Heterocycle.

The term “heterocycloalkoxy” refers to the radical -Alkoxy-Heterocycle (i.e., —O-Alkyl-Heterocycle).

The term “heteroaryloxy” refers to the radical —O-heteroaryl.

The term “heteroarylalkoxy” refers to the radical -Alkoxy-Heteroaryl (i.e., —O-Alkyl-Heteroaryl).

The term “alkylaminocarbonylamino” refers to the radical —NH—C(═O)—NH-Alkyl.

The term “alkanoyloxyalklyl” refers to the radical -alkyl-O(O═)C-alkly.

The term “alkylsulphonylamino” refers to the radical —NH—SO₂-Alkyl.

The term “alkylaminosulphonyl” refers to the radical —SO₂—NH-Alkyl.

The term “hydroxy” refers to the radical —OH.

A “metabolite” of a compound disclosed herein is a derivative of a compound which is formed when the compound is metabolised. The term “active metabolite” refers to a biologically active derivative of a compound that is formed when the compound is metabolised. The term “metabolised” refers to the sum of the processes by which a particular substance is changed in the living body. All compounds present in the body are manipulated by enzymes within the body in order to derive energy and/or to remove them from the body. Specific enzymes produce specific structural alterations to the compound. Cytochrome P450, for example, catalyzes a variety of oxidative and reductive reactions. Uridine diphosphate glucuronyltransferases, for example, catalyze the transfer of an activated glucuronic-acid molecule to aromatic alcohols, aliphatic alcohols, carboxylic acids, amines and free sulphhydryl groups. Further information on metabolism may be obtained from The Pharmacological Basis of Therapeutics, 9^{th} Edition, McGraw-Hill (1996), pages 11-17.

The metabolites of the compounds disclosed herein can be identified either by administration of compounds to a mammalian (e.g., human) host and analysis of tissue samples from the host, or by incubation of compounds with hepatic cells or other in vitro systems such as cytochromes or microsomes, and analysis of the resulting compounds. Both methods are well known in the art.

As used herein, the term “stercoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. As used herein, the term “optical isomer” is equivalent to the term “enantiomer”. Compounds that are stercoisomers of one another, but are not enantiomers of one another, are called diastereoisomers. The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ee = \frac{E1 - E2}{E1 + E2} * 100$$

wherein E1 is the amount of the first enantiomer and E2 is the amount of the second enantiomer. Thus, if the initial ratio of the two enantiomers is 50:50, such as is present in a racemic mixture, and an enantiomeric enrichment sufficient to produce a final ratio of 50:30 is achieved, the ee with respect to the first enantiomer is 25%. However, if the final ratio is 90:10, the ee with respect to the first enantiomer is 80%. In certain embodiments, the invention provides any of the compounds set forth above haveing an ee of greater than zero. More preferably, the compounds have an ee of greater than about 25%, or an ee of greater than about 50%. According to further embodiments of the invention, an ee of greater than about 80% or an ee of greater than about 90% is further preferred, an ee of greater than about 95% is still further preferred and an ee of greater than about 99% is most preferred. Enantiomeric enrichment is readily determined by one of ordinary skill in the art using standard techniques and procedures, such as high performance liquid chromatography with a chiral column. Choice of the appropriate chiral column, eluent and conditions necessary to effect separation of the enantiomeric pair is within the knowledge of one of ordinary skill in the art. In addition, the enantiomers of compounds of formula I can be resolved by one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization techniques or chiral chromatography.

Diastereisomers differ in both physical properties and chemical reactivity. A mixture of diastereomers can be separated into enantiomeric pairs based on solubility, fractional crystallization or chromatographic properties, e.g., thin layer chromatograph, column chromatography or HPLC.

Purification of complex mixtures of diastereomers into enantiomers typically requires two steps. In a first step, the mixture of diastereomers is resolved into enantiomeric pairs, as described above. In a second step, enantiomeric pairs are further purified into compositions enriched for one or the other enantiomer or, more preferably resolved into composition comprising pure enantiomers. Resolution of enantiomers typically requires reaction or molecular interaction with a chiral agent, e.g., solvent or column matrix. Resolution may be achieved, for example, by converting the mixture of enantiomers, e.g., a racemic mixture, into a mixture of diastereomers by reaction with a pure enantiomer of a second agent, i.e., a resolving agent. The two resulting diasteromeric products can then be separated. The separated diastereomers are then reconverted to the pure enantiomers by reversing the initial chemical transformation.

Resolution of enantiomers can also be accomplished by differences in their non-covalent binding to a chiral substance, e.g., by chromatography on homochiral absorbants. The noncovalent binding between enantiomers and the chromatographic adsorbant establishes diastereomeric complexes, leading to differential partitioning in the mobile and bound states in the chromatographic system. The two enantiomers therefore move through the chromatographic system, e.g, column, at different rates, allowing for their separation.

Chiral resolving columns are well known in the art and are commercially available (e.g., from MetaChem Technologies Inc., a division of ANSYS Technologies, Inc., Lake Forest, Calif.). Enantiomers can be analyzed and purified using, for example, chiral stationary phases (CSPs) for HPLC. Chiral HPLC columns typically contain one form of an enantiomeric compound immobilized to the surface of a silica packing material. For chiral resolution to occur, there must be at least three points of simultaneous interaction between the CSP and one analyte enantiomer, with one or more of these interactions being stereochemically dependent.

D-phenylglycine and L-leucine are Type I CSPs and use combinations of p-p interactions, hydrogen bonds, dipole-dipole interactions, and steric interactions to achieve chiral recognition. To be resolved on a Type I column, analyte enantiomers must contain functionality complementary to that of the CSP so that the analyte undergoes essential interactions with the CSP. The sample should preferably contain one of the following functional groups: p-acid or p-base, hydrogen bond donor and/or acceptor, or an amide dipole. Derivatization is sometimes used to add the interactive sites to those compounds lacking them. The most common derivatives involve the formation of amides from amines and carboxylic acids.

The MetaChiral ODM™ is a type II CSP. The primary mechanisms for the formation of solute-CSP complexes is through attractive interactions, but inclusion complexes also play an important role. Hydrogen bonding, pi-pi, and dipole stacking are important for chiral resolution on the MetaChiral™ ODM. Derivatization is often necessary when the solute molecule does not contain the groups required for solute-column interactions. Derivatization, usually to benzylamides, is also required of some strongly polar molecules like amines and carboxylic acids, which would otherwise interact too strongly with the stationary phase through non-stereo-specific interactions.

U.S. Pat. Nos. 5,346,896; 6,239,135; 6,358,958 and 6,514,976, and international publication number WO 94/15919, disclose compounds that bind to 5-HT_{1A} receptors. In certain embodiments, some or all of the compounds disclosed in the aforementioned patents and publication are excluded from formula I.

Preferred groups that R represent are a hydrogen or halogen atom or (C₁-C₆)-alkoxy, (C₁-C₆)-haloalkoxy, N,N-di-(C₁-C₆)-aminocarbonyl or cyano group. A preferred haloalkoxy the R is a polyhaloalkoxy, more preferably trifluoromethoxy. A preferred halogen atom that R represents is a fluorine atom. The preferred position for the aforementioned atoms and groups is on the 2-position of the phenyl group to which they are attached.

A preferred group that R₁ represents is a hydrogen atom.

Also preferred is where simultaneously, R represents one or more member selected from the groups consisting hydroxy, (C₁-C₆)-haloalkoxy, (C₁-C₆)-hydroxyalkyl, alkoxyalkyl, (C₁-C₆)-aminoalkyl, (C₁-C₆)-alkylamino-(C₁-C₆)-alkyl, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups; and R₁ represents a member selected from the group consisting of unsubstituted aryloxy, aralkyl, aralkoxy, heterocycloxy, heterocycloalkyl and heterocycloalkoxy groups, or a member selected from the group consisting of aryloxy, aralkyl, aralkoxy, heterocycloxy, heterocycloalkyl, heterocycloalkoxy, aryl, heterocyclic and cycloalkyl groups substituted with one or more substituent selected from the group consisting of R represents hydrogen or one or more substituents selected from the group consisting of (C₁-C₆)-alkylthio, hydroxy, (C₂-C₆)-alkenyl, (C₂-C₆)-alkynyl, (C₁-C₆)-haloalkoxy, (C₁-C₆-hydroxyalkyl, alkoxyalkyl, (C₁-C₆)-aminoalkyl, (C₁-C₆)-alkylamino-(C₁-C₆)-alkyl, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups.

Preferred groups that Q represents are —C(O)— and —CH(OR₂)— where R₂ represents a hydrogen atom or (C₁-C₆)-alkyl, (C₂-C₆)-alkenyl, (C₂-C₆)-alkynyl, —C(O)—(C₁-C₆)-alkyl, —C(O)O—(C₁-C₆)-alkyl, —C(O)NR₇R₈ or —C(S)NR₇R₈ wherein R₇ and R₈ are independently hydrogen or (C₁-C₆)-alkyl;

Preferred groups that R₃ represents are a hydrogen atom or a (C₁-C₆)-alkyl, (C₂-C₆)-alkenyl, (C₂-C₆)-alkynyl, cycloalkyl, aryl or heterocycle group. Also preferred is where R₃ represents hydrogen or a (C₁-C₆)-alkyl, (C₂-C₆)-alkenyl, (C₂-C₆)-alkynyl, each group being optionally substituted with one or more substituent R or R₁, defined as above. More preferably, R₃ represents a cyclohexyl group.

Preferred groups that R₄ represents are an aryl or heterocyclic group, each being optionally substituted with one or more substituent selected from the group consisting of halogen atom or (C₁-C₆)-alkoxy or (C₁-C₆)-haloalkoxy groups. A preferred halogen atom that is a substitutent on R₄ is fluorine. A preferred alkoxy group that is a substitutent on R₄ is a methoxy group. A preferred haloalkoxy group that is a sustitutent on R₄ is a polyhaloalkoxy group, most preferably a trifluoroethoxy group. A preferred aryl group that R₄ represents is a phenyl group. A preferred heterocyclic group that R₄ represents is a bicyclic heterocyclic group. More preferably R₄ represents a bicyclic heteroaryl group, most preferably a 2,3-dihydro-1,4-benzodioxinyl group.

Also preferred is where R₄ represents an aryl or heterocyclic group, substituted with one or more substituent selected from the group consisting of (C₁-C₆)-haloalkoxy, alkoxyalkyl, (C₁-C₆)-aminoalkyl, (C₁-C₆)-alkylamino-(C₁-C₆)-alkyl, acylamino, aminosulphonyl, (C₁-C₆)-alkylaminosulphonyl, cyano, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups.

A preferably represents a bond.

n is preferably 1.

Each of the foregoing preferences for formula I may be present independently or in any combination.

Also preferred are compounds of formula I wherein, simultaneously, R represents a hydrogen or halogent atom or (C₁-C₆)-alkoxy, (C₁-C₆)-haloalkoxy, N,N-di-(C₁-C₆)-aminocarbonyln or cyano group; R₁ represents is a hydrogen atom, Q represents —C(O)— or —CH(OR₂)— where R₂ represents a hydrogen atom or (C₁-C₆)-alkyl, (C₂-C₆)-alkenyl, (C₂-C₆)-alkynyl, —C(O)—(C₁-C₆)-alkyl, —C(O)O—(C₁-C₆)-alkyl, —C(O)NR₇R₈ or —C(S)NR₇R₈ wherein R₇ and R₈ are independently hydrogen or (C₁-C₆)-alkyl group; R₃ represents a hydrogen atom or a (C₁-C₆)-alkyl, (C₂-C₆)-alkenyl, (C₂-C₆)-alkynyl, cycloalkyl, aryl or heterocycle group; R₄ represents are an aryl or heterocyclic group, each being optionally substituted with one or more substituent selected from the group consisting of halogen atom or (C₁-C₆)-alkoxy or (C₁-C₆)-haloalkoxy groups; A represents a bond; and n=1.

Compounds of formula I can be separated into diastereomeric pairs by, for example, by separation by TLC. These diastereomeric pairs are referred to herein as diastereoisomer with upper TLC Rf; and diastereoisomer with lower TLC Rf. The diastereoisomers can further be enriched for a particular enantiomer or resolved into a single enantiomer using methods well known in the art, such as those described herein.

### SYNTHESIS OF THE COMPOUNDS OF THE INVENTION

The compounds of the invention are generally prepared according to the following schemes:

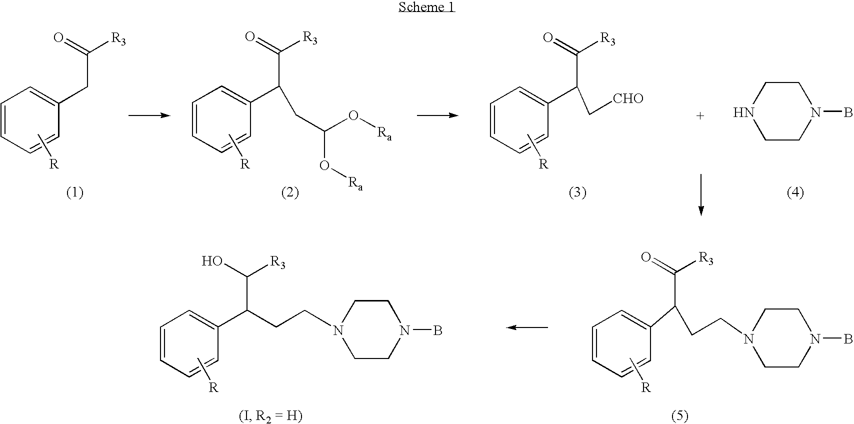

Groups B, R are the same as groups A−R₄, and (R+R₁) respectively, as given in the general formula I. R₂ and R₃ are the same as given in the general formula and each Rₐ is a protecting group, e.g., a lower alkyl group, or together form an alkylene chain.

Starting material (1) is treated with a base, preferably potassium tert-butoxide, followed by alkylation with 2-bromoacetaldehyde dialkyl acetal or other carbonyl protected 2-haloacetaldehyde (e.g., the Rₐ alkyl groups can also be joined in a cycle to give a dioxolane or dioxane ring). Other alternative and appropriate bases to carry out the condensation include lithium amides, sodium hydride, sodium hydroxide, potassium hydroxide, potassium carbonate, cesium carbonate and the like with the aid or not of phase transfer catalysts.

The reaction is preferably carried out in a solvent such as dimethyl sulfoxide or toluene at a temperature of 0° C. to reflux.

The use of 3-bromopropionaldehyde dialkyl acetal or other carbonyl protected 3-halopropionaldehyde allows to obtain, by following the same reaction conditions described above in Scheme 1, compound I having n=2 as foreseen in the general formula.

Treatment of (2) with an acid, such as hydrochloric acid or p-toluene-sulfonic acid or trifluoroacetic acid in a suitable organic solvent, achieves aldehyde (3). Generally, the reaction is conducted in a protic solvent, such a mixture of aqueous acid and acetone or tetrahydrofuran, at temperatures of from about 5° to 75° C. preferably at ambient temperature. A preferred and alike method consists of carrying out the reaction in a mixuture of aqueous trifluoroacetic acid in a chlorinated solvent at ambient temperature.

Aldehyde (3) is coupled with the desired aryl piperazine (4) by reductive amination procedure to prepare (5). The reaction is preferably conducted at ambient temperature in a non-reactive solvent such as dichloroethane or methylene chloride or chloroform in the presence of sodium triacetoxyborohydride and is substantially complete in one to 24 hours (see for example A. F. Abdel-Magid, et al., J. Org. Chem., 61, 3849 (1996)) or it can be conducted in a protic solvent (e.g., methanol) with the aid of sodium cyanoborohydride optionally in the presence of molecular sieves.

Reduction of (5) to the alcohol (I) is readily accomplished using a reducing agent such as sodium borohydride or, diisobutylaluminum hydride or other aluminum or boron hydride or other reduction method to carry out the conversion ketone to alcohol very well known to those skilled in the art, to prepare the hydroxy compound (I). The reaction is preferably conducted in an organic solvent such as methanol or methylene chloride or tetrahydrofuran at temperatures of from about −20° C. to ambient temperature.

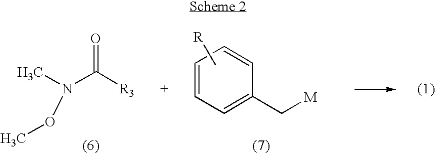

Starting material (1) is commercially available or can be prepared by coupling the proper Weinreb amide (6) (See, Nahm and Weinreb, Tetrahedron Lett., 22, 3815, (1981)) with (7), as described in Scheme 2 above, where M is a metallic salt, such as lithium or magnesium halide.

The reaction is preferably conducted under an inert atmosphere preferably nitrogen, in an aprotic solvent, such as tetrahydrofuran, at ambient or lower temperatures down to −78° C.

Alternatively, an ester of structure R₃COOalkyl can be treated with a substituted benzylmagnesium chloride or benzylmagnesium bromide or lithium derivative under standard conditions well known in the art to provide the ketone of structure (1).

An alternative way to obtain compounds (1) consists of reacting the proper arylaldehyde with an alkylnitro derivative in a nitroaldol fashion, dehydration of the nitro alcohol thus obtained, followed by double bond reduction to afford a 2-nitro(2-Ak)phenethyl derivative, which can undergo Nef reaction to yield the desired keto derivative 1. This kind of pathway is well documented in the experimental part and in the literature.

Preferred and alike way of synthesis of (1) is the palladium catalyzed coupling of an acyl halide with a compound (7) where M is Zn halide.

More specifically, the compounds of formula (5) can be prepared following the procedure described in Scheme 3. All substituents, unless otherwise indicated, are as defined previously. The reagents and starting materials are readily available to one of ordinary skill in the art.

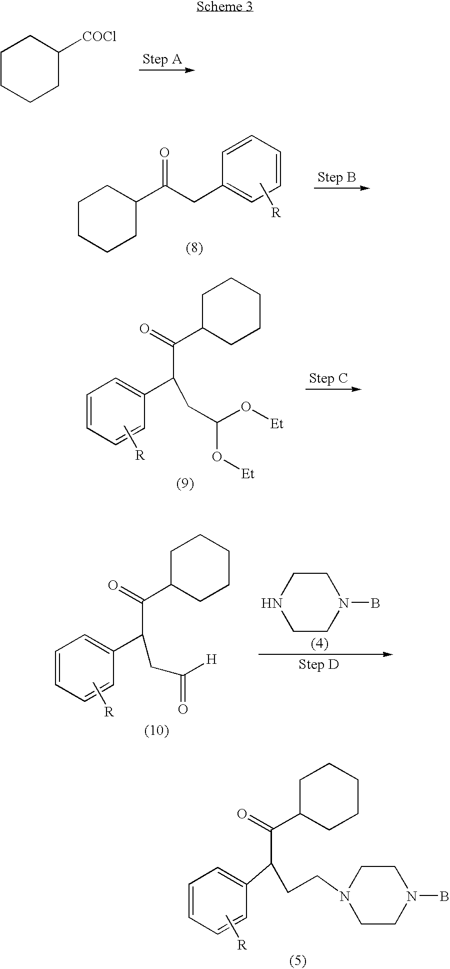

In Scheme 3, step A, for example, cyclohexanecarbonyl chloride is added to a mixture of the suitable benzylzinc chloride or bromide and a proper palladium catalyst, e.g., dichlorobis(triphenylphosphine)palladium (II) stirred at 0° C. in a solvent such as tetrahydrofuran. Afterwards, stirring is continued at ambient temperature for 4-24 h. Then the reaction is quenched for example with an aqueous saturated solution of ammonium chloride. Usual work-up procedure by extraction provide the ketone (8). Ketone (8) can be purified by techniques well known in the art, such as flash chromatography on silica gel with a suitable eluent, such as ethyl acetate/hexane to provide the purified material. Alternatively, the crude ketone (8) can be carried on to step B.

In Scheme 3, step B, ketone (8) is alkylated with bromoacetaldehyde diethyl acetal under conditions well known in the art to provide compound of structure (9). For example, ketone (8) is dissolved in a suitable organic solvent, such as dimethyl sulfoxide or toluene and treated with a slight excess of a suitable base, such as potassium tert-butoxide. The reaction is stirred for about 15 to 30 minutes at a temperature of between 0° C. and the reflux temp. of the solvent and bromoacetaldehyde diethyl acetal is added dropwise to the reaction. One of ordinary skill in the art would readily appreciate that bromoacetaldehyde dimethyl acetal, bromoacetaldehyde ethylene acetal and the like may be used in place of the corresponding diethyl acetal.

In Scheme 3, step C, compound (9) is hydrolyzed under acidic conditions to provide aldehyde (10) in a manner analogous to the procedure described in Scheme I. More specifically, for example, compound (9) is dissolved in a suitable organic solvent, such as dichloromethane and treated with a suitable acid, such as aq. trifluoroacetic acid. The reaction mixture is stirred for about 1 to 6 hours at room temperature. The reaction mixture is then diluted with the same solvent, washed with brine, the organic layer is separated, dried over anhydrous sodium sulfate, filtered and concentrated under vacuum to provide aldehyde (10). Aldehyde (10) can be purified by techniques well known in the art, such as flash chromatography on silica gel with a suitable eluent, such as ethyl acetate/hexane. Alternatively, crude aldehyde (10) can be used directly in step D.

In Scheme 3, step D, aldehyde (10) is reductively aminated, under conditions well known in the art, with piperazine (4) to provide the ketone (5) in a manner analogous to the procedure described in Scheme I. More specifically, for example, aldehyde (10) is dissolved in a suitable organic solvent, such as methylene chloride. To this solution is added about 1.05 or more equivalents of piperazine (4). Acetic acid may optionally be added to aid in dissolution of the piperazine (4). Then about 1.4 to 1.5 equivalents of sodium triacetoxyborohydride is added and the reaction is stirred at room temperature for about 3 to 5 hours. The reaction is then quenched by addition of a suitable base, such as aqueous sodium carbonate or hydroxide to provide a pH of about 8 to about 12. The quenched reaction is then extracted with a suitable organic solvent, such as methylene chloride. The organic extracts are combined, washed with brine, dried, filtered and concentrated under vacuum to provide the compound of formula (5). This material can then be purified by techniques well known in the art, such as flash chromatography on silica gel with a suitable eluent, such as ethyl acetate/petroleum ether or hexane.

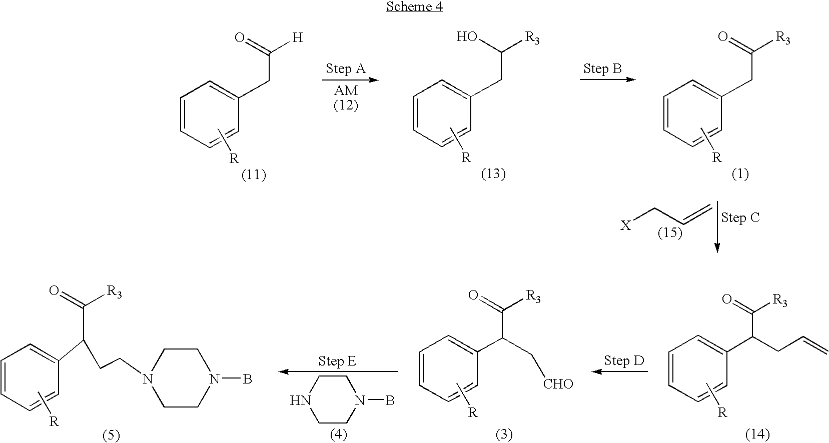

Alternatively, compounds of structure (5) can be prepared following the procedure described in Scheme 4. All substituents, unless otherwise indicated, are defined previously. The reagents and starting materials are readily available to one of ordinary skill in the art.

In Scheme 4, step A, aldehyde (11) is combined with a suitable organometallic reagent (12) under conditions well known in the art to provide alcohol (13). Examples of suitable organometallic reagents include Grignard Reagents, alkyl lithium reagents, alkyl zinc reagents, and the like. Grignard Reagents are preferred. For examples of typical Grignard Reagents and reaction conditions, see J. March, “Advanced Organic Chemistry: Reactions, Mechanisms, and Structure”, 2nd Edition, McGraw-Hill, pages 836-841 (1977). More specifically, aldehyde (11) is dissolved in a suitable organic solvent, such as tetrahydrofuran or toluene, cooled to about −5° C. and treated with about 1.1 to 1.2 equivalents of a Grignard reagent of formula (12) wherein M is MgCl or MgBr. The reaction is stirred for about 0.5 to 6 hours, then quenched, and alcohol (13) is isolated by well-known work-up procedure.

In Scheme 4, step B, alcohol (13) is oxidized under standard conditions well know in the art, such as those described by J. March, “Advanced Organic Chemistry: Reactions, Mechanisms, and Structure”, 2nd Edition, McGraw-Hill, pages 1082-1084 (1977), to provide ketone (1). (Ketone (1) is the starting material used in Scheme 1 above.)

The oxidation can also be performed using standard Swem Oxidation conditions which are well known to one of ordinary skill in the art (Marx,Tidwell—J. Org. Chem. 49,788 (1984), or the alcohol (13) is dissolved in a suitable organic solvent, such as methylene chloride, the solution cooled with a wet ice-acetone bath, and treated with 2.5 to 3.0 equivalents of dimethyl sulfoxide. After stirring for about 30 minutes, the reaction is then treated with about 1.8 equivalents of P₂O₅. The reaction is stirred for about 3 hours and then, preferably, treated over about 30 minutes with about 3.5 equivalents of a suitable amine, such as triethylamine. The cooling bath is then removed and the reaction is stirred for about 8 to 16 hours. The ketone (1) is then isolated by standard extraction techniques well known in the art.

In Scheme 4, step C, ketone (1) is treated with a suitable base followed by addition of the alkene (15), wherein X is a suitable leaving group, to provide compound (14). For example, ketone (1) is combined with an excess of alkene (15) in a suitable organic solvent, such as tetrahydrofuran, and cooled with a wet ice acetone bath. Examples of suitable leaving groups are Cl, Br, I, tosylate, mesylate, and the like. Preferred leaving groups are Cl and Br. About 1.1 equivalents of a suitable base is added and the reaction is allowed to stir for about 2 hours at room temperature. Examples of suitable bases are potassium tert-butoxide, sodium hydride, NaN(Si(CH₃)₃)₂, LDA, KN(Si(CH₃)₃)₂, NaNH₂, sodium ethoxide, sodium methoxide and the like. Potassium tert-butoxide is the preferred suitable base. The reaction is then quenched with aqueous acid and compound (14) is isolated by usual work-up procedure.

In Scheme 4, step D, compound (14) is treated with a suitable oxidizing agent to provide aldehyde (3). (Aldehyde (3) is also prepared in Scheme 1) Examples of suitable oxidizing agents are ozone, NaIO₄/Osmium catalyst, and the like. Ozone is the preferred oxidizing agent. Examples of suitable oxidizing reagents and conditions are described by J. March, “Advanced Organic Chemistry: Reactions, Mechanisms, and Structure”, 2nd Edition, McGraw-Hill, pages 1090-1096 (1977).

For example, compound (14) is dissolved in a suitable organic solvent, such as methanol, a small amount of Sudan III is added, and the solution is cooled to about −20° C. Ozone is bubbled into the solution for about 4 hours until the pink color turns to a pale yellow color. Then a reducing agent such as Me₂ S or tributylphosphine is added. Concentration provides the intermediate dimethyl acetal of aldehyde (3). This dimethyl acetal is readily hydrolyzed under standard acidic conditions to provide aldehyde (3). Alternatively, direct acidic work-up of the crude reaction mixture provides aldehyde (3). Alternatively, aldehyde (3) can be obtained directly by ozonolysis of (14) in a non-acetal forming solvent, such as methylene chloride.

In Scheme 4, step E, aldehyde (3) is reductively aminated under conditions analogous to those described above in Scheme 3, step D, to provide compound (5). (Compound 5 is also prepared in Scheme I)

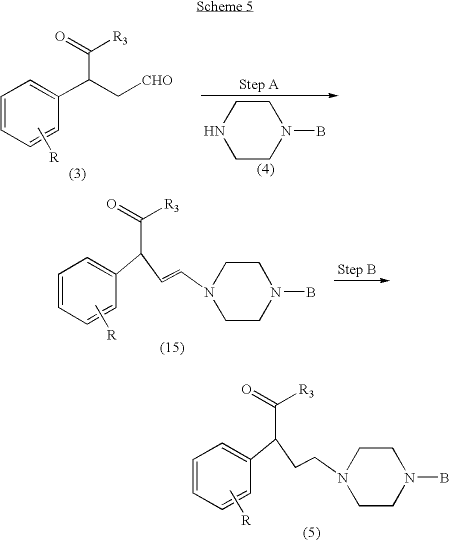

Scheme 5 provides an alternative synthesis for the preparation of ketone (5). All substituents, unless otherwise indicated, are as defined previously. The reagents and starting materials are readily available to one of ordinary skill in the art.

In Scheme 5, step A, aldehyde (3) is condensed with piperazine (4) under standard conditions well known in the art to provide the enamine (15). For example, about 1.05 equivalents of aldehyde (3) dissolved in a suitable organic solvent, such as isopropyl acetate or isopropanol, is added to neat piperazine (4), free base. Additional organic solvent is added to produce a slurry and the reaction is stirred for about 1 to 2 hours. The enamine (15) is then isolated by standard techniques, such as collection by filtration.

In Scheme 5, step B, the enamine (15) is hydrogenated under conditions well known to one of ordinary skill in the art to provide compound (5). For example, enamine (15) is combined with a suitable organic solvent, such as isopropyl alcohol and a catalytic amount of 5% palladium on carbon in a Parr bottle. The mixture is placed under 50 psi of hydrogen and shaken for about 2 days at room temperature. The slurry is then filtered to remove catalyst and the filtrate is concentrated to provide compound (5).

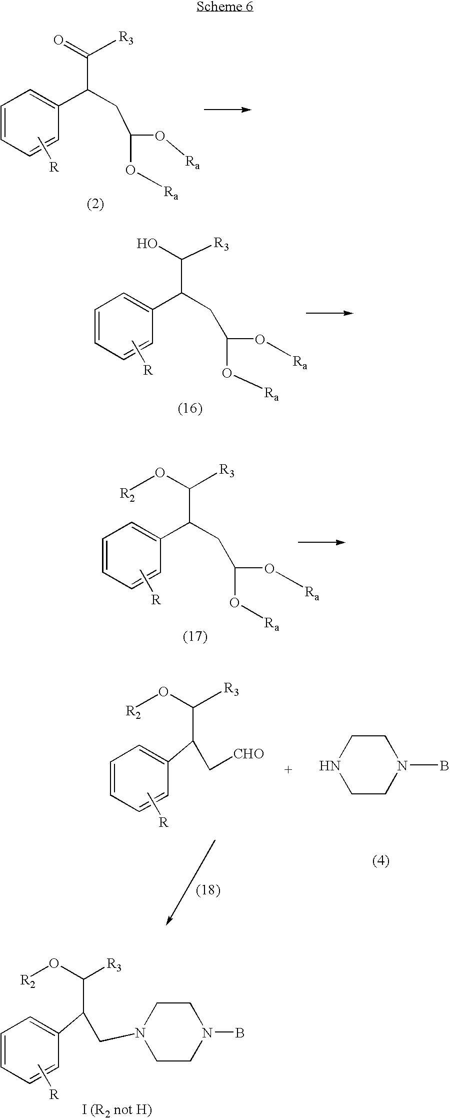

For the synthesis of compounds I where R₂ is different than H, the method given in Scheme 6 is used. Intermediate ketone (2) is reduced with the same reduction methods used above in scheme 1 for compound (5) affording intermediate (16), which is etherified by reaction with a base, for example NaH or potassium tert-butoxide or NaNH₂ or LiNH₂ or others in a suitable solvent e.g. tetrahydrofuran, affording the alkoxide, which is then reacted in situ with the proper R₂-X with X leaving group (halogen or mesylate or tosylate) and R₂=lower alkyl at a temperature of from 0° C. to the reflux temperature. The obtained compounds (17) can undergo the same reactions described in scheme 1 affording product I with R₂ not H.

Alternatively, compounds of formula I where R₂ is not a hydrogen atom, can be obtained by alkylating compounds of formula I where R₂=H with the same methods described above for alkylating compound 16, limiting this procedure to the alkylation with very reactive halogenide or mesylate/tosylate (e.g. benzyl bromides) which can react under time/temperature controlled reaction condition, preferably at r.t.

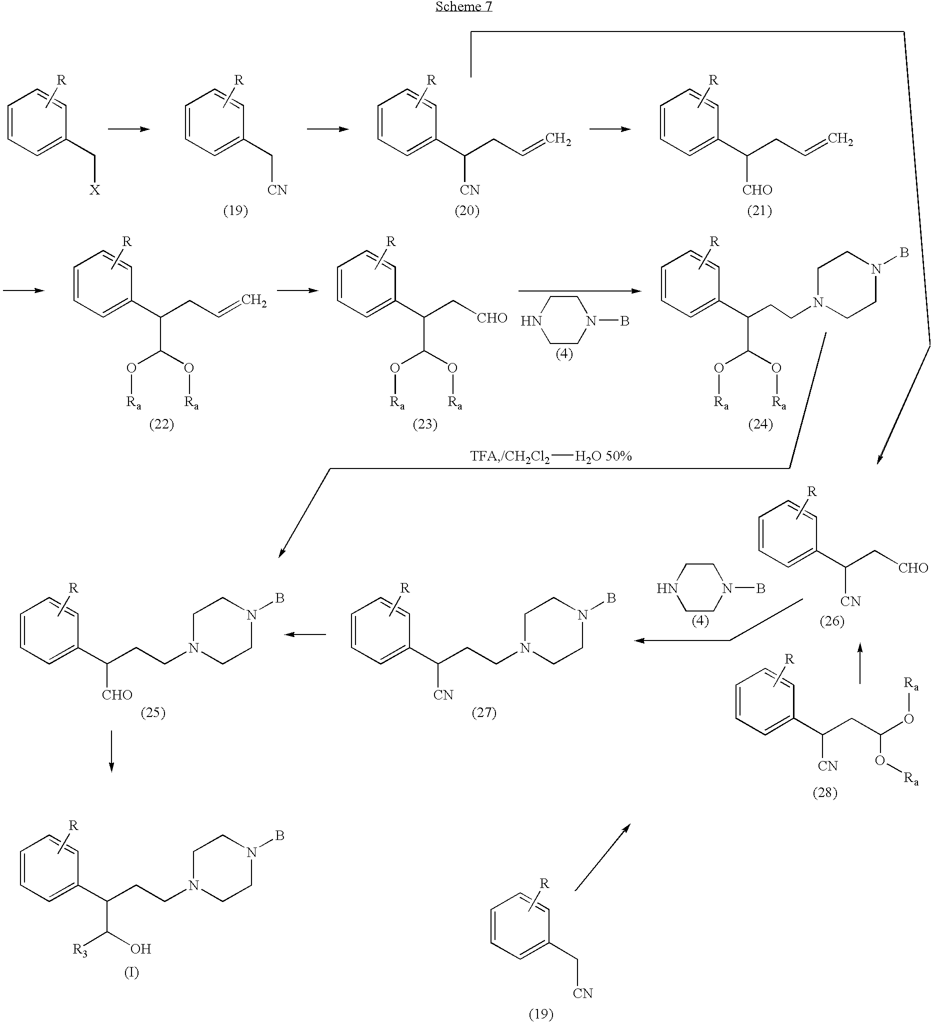

Scheme 7 describes a double functionalization approach to the synthesis of Compound (I). This kind of approach can be useful for the synthesis of libraries of compounds (I) introducing different piperazine moieties and different R₃ groups at the same time.

Compounds of formula (I), where R₂ represents —C(O)Alk, —C(O)OAlk, —C(O)NR₇R₈ or —C(S)NR₇R₈ can be obtained by alkylation or addition reactions starting from compounds of formula (I) where R₂=H. These kinds of reactions can be carried out using proper acyl halides, alkyl chloroformates, isocyanates or isothiocyanates in methylene chloride, pyridine or DMF, optionally in the presence of a base such as TEA or NaH, or alternatively (e.g., for isothiocyanates) of an acid such as trifluoroacetic acid, at a temperature range of r.t. −80° C.

In scheme 7 groups B and R are the same as groups A−R₄, and (R+R₁) respectively, as given in the general formula I; R₂ and R₃ are the same as given in the general formula and Rₐ is a protecting group, e.g., is a lower alkyl group or the two Rₐ groups are linked forming a 1,3-dioxolanyl or 1,3-dioxanyl group.

A proper commercial benzyl derivative (with X=halogen or methanesulphonyloxy or p-toluenesulphonyloxy groups) can be reacted, as well known to those skilled in the art, to afford the benzyl cyanide (19). These reactants can be converted following known alkylation methods into compounds (20) or (28) respectively reacting them with allyl halogenides (or allyl mesylates or tosylates) or haloalkylaldehydes in their carbonyl protected form (acetals or dioxolanyl derivatives or other).

These alkylation reactions can be carried out by the use of bases to generate the reactive benzyl carbanions. Example of used bases are lithium diisopropylamide (LDA) or tert-Butyl lithium or NaH or potassium tert-butoxide or sodium amide or potassium amide or others in a proper solvent such as THF or Et₂O or DMF or other at a temperature ranging from −78° C. to the reflux temperature. A preferred method of alkylation includes the use of hindered bases such as LDA in the presence of hexamethyl phosphorous triamide or DMPU at −78° C.—r.t.

Compounds (20) can be in turn reduced by the use of diisobutylaluminum hydride (DIBAL-H) in a proper solvent (toluene, DMF, CH₂Cl₂ or other) at a temperature ranging from −78° C. to the reflux of the solvent. The so obtained aldehydes (21) are then carbonyl protected following methods very well known to those skilled in the art to give compounds (22), which can be catalytically osmilated (C. P. Forbes J. C. S. Perkin Trans I, 1979, 906-910) or undergo ozonolysis to afford compounds (23). Compounds (23) can be reductively aminated as described above to afford compounds (24). Deprotection by common methods leads to the aldehydes (25).

Compounds (25) can be alternatively obtained from compounds (20) applying the osmilation or ozonolysis procedure on them. The cyanopropionaldehydes (26) thus obtained are then reductively aminated to compound (27). Repeating the DIBAL-H reduction described above on these compounds affords the aldehydes (25).

Compounds (26) are also easily obtained from compounds (28) by simple deprotection of the carbonyl functionality.

The reaction of R₃-M (where M is a metallic salt, such as lithium or magnesium halide) with compounds (25) afford compounds (I). A large number of organometallics such as lithium or magnesium derivatives are commercially available or easily prepared and can be reacted in a proper solvent such as THF or Et₂O or others at −78° C. —reflux.

### Stereochemistry

In Schemes 1, 6 and 7 compounds I are obtained in syn/anti mixture of diastereoisomers with ratio depending on the reaction condition used. The diastereoisomers can be separated by usual techniques known to those skilled in the art including fractional crystallization of the bases or their salts or chromatographic techniques such as LC or flash chromatography. For both of the diastereoisomers, the (+) enantiomer of formula Ia can be separated from the (−) enantiomer using techniques and procedures well known in the art, such as that described by J. Jacques, et al., “Enantiomers, Racemates, and Resolutions”, John Wiley and Sons, Inc., 1981. For example, chiral chromatography with a suitable organic solvent, such as ethanol/acetonitrile and Chiralpak AD packing, 20 micron can also be utilized to effect separation of the enantiomers.

The free bases of formula I, their diastereoisomers or enantiomers can be converted to the corresponding pharmaceutically acceptable salts under standard conditions well known in the art. For example, the free base of formula I is dissolved in a suitable organic solvent, such as methanol, treated with one equivalent of maleic or oxalic acid for example, one or two equivalents of hydrochloric acid or methanesulphonic acid for example, and then concentrated under vacuum to provide the corresponding pharmaceutically acceptable salt. The residue can then be purified by recrystallization from a suitable organic solvent or organic solvent mixture, such as methanol/diethyl ether.

The N-oxides of compounds of formula I can be synthesized by simple oxidation procedures well known to those skilled in the art. The oxidation procedure described by P. Brougham et al. (Synthesis, 1015-1017, 1987), allows the two nitrogen of the piperazine ring to be differentiated, enabling both the N-oxides and N,N′-dioxide to be obtained.

### Combination Treatments

In certain embodiments, disorders of the urinary tract are treated by administering a compound of formula I in combination with an additional 5-HT_{1A} antagonist or an antagonist of one or more additional class of receptors. In preferred embodiments a compound of formula I is administered in combination with an antagonist of an α1-adrenergic, or muscarinic receptor.

In further embodiments, lower urinary tract disease is treated by administering a compound of formula I in combination with one or more inhibitor of the cyclooxygenase enzyme, which may inhibit both COX1 and COX2 isozymes or which may, alternatively, be selective for COX2 isozyme, and NO donor derivatives thereof.

Examples of antimuscarinic drugs for administration in combination with a compound of formula I are oxybutynin, tolterodine, darifenacin, and temiverine.

A compound of formula I may be administered in combination with α1-adrenergic antagonists, for the therapy of lower urinary tract symptoms, whether or not these are associated with BPH. Preferred α1-adrenergic antagonists suitable for administration in combination with a compound of formula I are, for example, prazosin, doxazosin, terazosin, alfuzosin, and tamsulosin. Additional α1-adrenergic antagonists suitable for administration in combination with a compound of formula I are described in U.S. Pat. Nos. 5,990,114; 6,306,861; 6,365,591; 6,387,909; and 6,403,594.

Examples of 5-HT_{1A} antagonists that may be administered in combination with a compound of formula I are found in Leonardi et al., J. Pharmacol. Exp. Ther. 299: 1027-1037, 2001 (e.g., Rec 15/3079), U.S. Pat. No. 6,071,920, other phenylpiperazine derivatives described in WO 99/06383 and pending U.S. patent applications Ser. No. 10/266,088 and 10/266,104 filed on Oct. 7, 2002. Additional 5-HT_{1A} antagonists include DU-125530 and related compounds described in U.S. Pat. No. 5,462,942 and robalzotan and related compounds described in WO 95/11891.

Examples of selective COX2 inhibitors that may be administered in combination with a compound of formula I are, without limitation, nimesulide, meloxicam, rofecoxib, celecoxib, parecoxib and valdecoxib. Additional examples of selective COX2 inhibitors are described, without limitation, in U.S. Pat. No. 6,440,963. Examples of non-selective COX1-COX2 inhibitors are, without limitation, acetylsalicylic acid, niflumic acid, flufenamic acid, enfenamic acid, meclofenamic acid, tolfenamic acid, thiaprophenic acid, ibuprofen, naproxen, ketoprofen, flurbiprofen, furprofen, indomethacin, acemethacin, proglumethacin, ketorolac, diclofenac, etodolac, sulindac, fentiazac, tenoxicam, lomoxicam, cynnoxicam, ibuproxam, nabumetone, tolmetin, amtolmetin. Accordingly, each of the foregoing are non-limiting examples of COX inhibitors that may be administered in combination with a compound of formula I.

Examples of derivatives of COX inhibitors that may be administered in combination with a compound of formula I are derivatives of COX inhibitors bearing nitrate (nitrooxy) or nitrite groups, such as those given, for example, in WO 98/09948, able to release NO in vivo.

### Pharmaceutical Compositions

The invention further provides pharmaceutical compositions comprising a compound of formula I or an enantiomer, diastereomer, N-piperazine oxide, crystalline form, hydrate, solvate, active metabolite or pharmaceutically acceptable salt of the compound. The pharmaceutical composition may also include optional additives, such as a pharmaceutically acceptable carrier or diluent, a flavouring, a sweetener, a preservative, a dye, a binder, a suspending agent, a dispersing agent, a colorant, a disintegrator, an excipient, a diluent, a lubricant, an absorption enhancer, a bactericide and the like, a stabiliser, a plasticizer, an edible oil, or any combination of two or more of said additives.

Suitable pharmaceutically acceptable carriers or diluents include, but are not limited to, ethanol, water, glycerol, aloe vera gel, allantoin, glycerine, vitamin-A and E oils, mineral oil, phosphate buffered saline, PPG2 myristyl propionate, magnesium carbonate, potassium phosphate, vegetable oil, animal oil and solketal.

Suitable binders include, but are not limited to, starch, gelatine, natural sugars such as glucose, sucrose and lactose, corn sweeteners, natural and synthetic gums such as acacia, tragacanth, vegetable gum, sodium alginate, carboxymethylcellulose, polyethylene glycol, waxes and the like.

Suitable disintegrators include, but are not limited to, starch such as corn starch, methyl cellulose, agar, bentonite, xanthan gum and the like.

Suitable lubricants include, but are not limited to, sodium oleate, sodium stearate, magnesium stearate, sodium benzoate, sodium acetate, sodium chloride and the like.

Suitable suspending agents include, but are not limited to, bentonite.

Suitable dispersing and suspending agents include, but are not limited to, synthetic and natural gums such as vegetable gum, tragacanth, acacia, alginate, dextran, sodium carboxymethylcellulose, methylcellulose, polyvinylpyrrolidone and gelatine.

Suitable edible oils include, but are not limited to, cottonseed oil, sesame oil, coconut oil and peanut oil.

Examples of additional additives include, but are not limited to, sorbitol, talc, stearic acid and dicalcium phosphate.

### Unit Dosage Forms

The pharmaceutical composition may be formulated as unit dosage forms, such as tablets, pills, capsules, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. The unit dosage forms may be used for oral, parenteral, intranasal, sublingual or rectal administration, or for administration by inhalation or insufflation, transdermal patches, and a lyophilized composition. In general, any delivery of active ingredients that results in systemic availability of such ingredients can be used. Preferably the unit dosage form is an oral dosage form, most preferably a solid oral dosage; therefore the preferred dosage forms are tablets, pills and capsules. However, parenteral preparations are preferred too.

Solid unit dosage forms may be prepared by mixing the active agents of the present invention with a pharmaceutically acceptable carrier and any other desired additives as described above. The mixture is typically mixed until a homogeneous mixture of the active agents of the present invention is obtained and the carrier and any other desired additives are formed, i.e. the active agents are dispersed evenly throughout the composition. In this case, the composition can be formed as dry or moist granules.

Dosage forms can be formulated as, for example, “immediate release” dosage forms. “Immediate release” dosage forms are typically formulated as tablets that release at least 60%-90% of the active ingredient within 30-60 min when tested in a drug dissolution test, e.g., U.S. Pharmacopeia standard <711>. In a preferred embodiment, immediate dosage forms release at 75% of active ingredient within about 45 min.

Dosage forms can also be formulated as, for example, “controlled release” dosage forms. “Controlled,” “sustained,” “extended” or “time release” dosage forms are equivalent terms that describe the type of active agent delivery that occurs when the active agent is released from a delivery vehicle at an ascertainable and manipulatable rate over a period of time, which is generally on the order of minutes, hours or days, typically ranging from about sixty minutes to about 3 days, rather than being dispersed immediately upon entry into the digestive tract or upon contact with gastric fluid. A controlled release rate can vary as a function of a multiplicity of factors. Factors influencing the rate of delivery in controlled release include the particle size, composition, porosity, charge structure, and degree of hydration of the delivery vehicle and the active ingredient(s), the acidity of the environment (either internal or external to the delivery vehicle), and the solubility of the active agent in the physiological environment, i.e., the particular location along the digestive tract. Typical parameters for dissolution test of controlled release forms are found in U.S. Pharmacopeia standard <724>.

Dosage forms can also be formulated to deliver active agent in multiphasic stages whereby a first fraction of an active ingredient is released at a first rate and at least a second fractions of active ingredient is released at a second rate. In a preferred embodiment, a dosage form can be formulated to deliver active agent in a biphasic manner, comprising a first “immediate release phase”, wherein a fraction of active ingredient is delivered at a rate set forth above for immediate release dosage forms, and a second “controlled release phase,” wherein the remainder of the active ingredient is released in a controlled release manner, as set forth above for controlled release dosage forms.

Tablets or pills can be coated or otherwise prepared so as to form a unit dosage form that has delayed and/or sustained action, such as controlled release and delayed release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the duodenum or to be delayed in release.

Biodegradable polymers for controlling the release of the active agents include, but are not limited to, polylactic acid, polyepsilon caprolactone, polyhydroxybutyric acid, polyorthoesters, polyacetals, polydihydropyrans, polycyanoacrylates and crosslinked or amphipathic block copolymers of hydrogels.

For liquid dosage forms, the active substances or their physiologically acceptable salts are dissolved, suspended or emulsified, optionally with the usually employed substances such as solubilizers, emulsifiers or other auxiliaries. Solvents for the active combinations and the corresponding physiologically acceptable salts can include water, physiological salt solutions or alcohols, e.g. ethanol, propanediol or glycerol. Additionally, sugar solutions such as glucose or mannitol solutions may be used. A mixture of the various solvents mentioned may be used in the present invention too.

A transdermal dosage form is contemplated by the present invention too. Transdermal forms may be a diffusion transdermal system (transdermal patch) using either a fluid reservoir or a drug-in-adhesive matrix system. Other transdermal dosage forms include, but are not limited to, topical gels, lotions, ointments, transmucosal systems and devices, and iontophoretic (electrical diffusion) delivery systems. Transdermal dosage forms may be used for delayed release and sustained release of the active agents of the present invention.

The pharmaceutical compositions and unit dosage forms of the present invention for parenteral administration, and in particular by injection, typically include a pharmaceutically acceptable carrier, as described above. A preferred liquid carrier is vegetable oil. Injection may be, for example, intravenous, epidural, intrathecal, intramuscular, intraluminal, intratracheal or subcutaneous.

The active agents can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active agents of the present invention may also be coupled with soluble polymers such as targetable drug carriers. Such polymers include, but are not limited to, polyvinylpyrrolidone, pyran copolymers, polyhydroxypropylmethacrylamidophenol, polyhydroxyethylaspartamidophenol, and polyethylenoxypolylysine substituted with palmitoyl residues.

### Administration

The pharmaceutical composition or unit dosage forms of the present invention may be administered by a variety of routes, such as the oral and enteral, intravenous, intramuscular subcutaneous, transdermal, transmucosal (including rectal and buccal) and by inhalation routes. Oral or transdermal routes are preferred (e.g., solid or liquid formulations or skin patches, respectively).

The pharmaceutical composition or unit dosage forms comprising an effective amount of the present invention may be administered to an animal, preferably a human, in need of treatment of neuromuscular dysfunction of the lower urinary tract described by E. J. McGuire in “Campbell's UROLOGY”, 5^{th} Ed., 616-638, 1986, W. B. Saunders Company, and patients affected by any physiological dysfunction related to impairment of 5-HT_{1A} receptor function. Such dysfunctions include, without limitation, central-nervous-system disorders such as depression, anxiety, eating disorders, sexual dysfunction, addiction and related problems.

As used herein, the term “effective amount” refers to an amount that results in measurable amelioration of at least one symptom or parameter of a specific disorder. In a preferred embodiment, the compound treats disorders of the urinary tract, such as urinary urgency, overactive bladder, increased urinary frequency, reduced urinary compliance (reduced bladder storage capacity), cystitis (including interstitial cystitis), incontinence, urine leakage, enuresis, dysuria, urinary hesitancy and difficulty in emptying the bladder, or central nervous system disorders due to serotonergic dysfunction (such as anxiety, depression, hypertension, sleep/wake cycle disorders, feeding behaviour, sexual function and cognition disorders in mammals (particularly a human) associated to stroke, injury, dementia and due to neurological development, disorders from hyperactivity related to an attention deficit (ADHD), drug addiction, drug withdrawal, irritable bowel syndrome.

The pharmaceutical composition or unit dosage form of the present invention may be administered according to a dosage and administration regimen defined by routine testing in the light of the guidelines given above in order to obtain optimal activity while minimising toxicity or side effects for a particular patient. However, such fine tuning of the therapeutic regimen is routine in the light of the guidelines given herein.

The dosage of the active agents of the present invention may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

For example, a single patient may suffer from several symptoms of dysuria simultaneously, such as, for example, urgency and excessive frequency of urination or both, and these may be reduced using the methods of the present invention. In the case of incontinence, any reduction in the frequency or volume of unwanted passage of urine is considered a beneficial effect of the present method of treatment.

The amount of the agent to be administered can range between about 0.01 and about 25 mg/kg/day, preferably between about 0.1 and about 10 mg/kg/day and most preferably between 0.2 and about 5 mg/kg/day. It will be understood that the pharmaceutical formulations of the present invention need not necessarily contain the entire amount of the agent that is effective in treating the disorder, as such effective amounts can be reached by administration of a plurality of doses of such pharmaceutical formulations.

In a preferred embodiment of the present invention, the compounds are formulated in capsules or tablets, preferably containing 50 to 200 mg of the compounds of the invention, and are preferably administered to a patient at a total daily dose of 50 to 400 mg, preferably 150 to 250 mg and most preferably about 200 mg, for relief of urinary incontinence and dysfunctions under treatment with 5-HT_{1A} receptor ligand.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents versus 100% total weight of the dosage form.

The pharmaceutical composition or unit dosage form may be administered in a single daily dose, or the total daily dosage may be administered in divided doses. In addition, co-administration or sequential administration of another compound for the treatment of the disorder may be desirable. For example, the compounds of the invention may be administered in combination with more antimuscarinic, α₁-adrenergic antagonist, 5-HT_{1A} receptor antagonist, or COX inhibitors or NO releasing derivatives thereof, for the therapy of lower urinary tract symptoms. Examples of antimuscarinics, α₁-adrenergic antagonists, 5-HT_{1A} receptor antagonist, COX inhibitors and NO releasing derivatives thereof are set forth above, without limitation.

For combination treatment where the compounds are in separate dosage formulations, the compounds can be administered concurrently, or each can be administered at separate staggered times. For example, the compound of the invention may be administered in the morning and the antimuscarinic compound may be administered in the evening, or vice versa. Additional compounds may be administered at specific intervals too. The order of administration will depend upon a variety of factors including age, weight, sex and medical condition of the patient; the severity and aetiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

### Uses-Methods for Treatment

Without wishing to be bound by theory, it is believed that administration of 5-HT_{1A} receptor antagonists prevents unwanted activity of the sacral reflex and/or cortical mechanisms that control micturition. Thus, it is contemplated that a wide range of neuromuscular dysfunctions of the lower urinary tract can be treated using the compounds of the present invention, including without limitation dysuria, incontinence and enuresis (overactive bladder). Dysuria includes urinary frequency, nocturia, urgency, reduced urinary compliance (reduced bladder storage capacity), difficulty in emptying the bladder, i.e. a suboptimal volume of urine is expelled during micturition. Incontinence syndromes include stress incontinence, urgency incontinence and enuresis incontinence, as well as mixed forms of incontinence. Enuresis refers to the involuntary passage of urine at night or during sleep.

The compounds of the present invention may also be useful for the treatment of central nervous system disorders due to serotonergic dysfunction.

The following examples represent typical syntheses of the compounds of formula I as described generally above. These examples are illustrative only and are not intended to limit the invention in any way. The reagents and starting materials are readily available to one of ordinary skill in the art.

### EXAMPLE 1

### 1-[4-Cyclohexyl-3-(2-fluorophenyl)-4-methoxybutyl]-4-[2-(2,2,2-trifluoroethoxy)phenyl]piperazine

1-Cyclohexyl-2-(2-fluoronhenyl)ethanone (Compound 1a)

To a mixture of 36 ml of 2-fluorobenzylzinc chloride (0.5 M sol. in THF) and 0.008 g of dichlorobis(triphenylphosphine)palladium (II) stirred at 0° C. was added dropwise via a syringe 2.14 ml of cyclohexanecarbonyl chloride. Afterwards, the reaction mixture was stirred at r.t. for 4 h, quenched with an aqueous saturated solution of ammonium chloride (25 ml), extracted with 20 ml of EtOAc, which was dried (Na₂SO₄) and evaporated to dryness in vacuo affording 3.52 g of the title compound as a crude, which could be used in the following step without further purification.

¹H-NMR (CDCl₃, δ): 1.10-2.05 (m, 10H), 2.47 (tt,1H), 3.77 (s, 2H), 6.97-7.32 (m, 4H)

4-Cyclohexyl-4-oxo-3-(2-fluorophenyl)butyraldehyde diethyl acetal (Compound 1b)

A solution of 5.02 g of compound I a in 136 ml of toluene was heated at reflux recovering 35 ml of toluene by distillation to remove water. Afterwards, 3.18 g of potassium tert-butoxide was added and stirring at reflux was continued for 30 min.; the reaction mixture was cooled to 80° C. and 4.27 ml of 2-bromoacetaldehyde diethyl acetal was added. After 18 h at reflux, the reaction mixture was cooled to r.t., quenched with an aqueous saturated solution of ammonium chloride (30 ml), extracted with 30 ml of EtOAc, which was dried (Na₂SO₄) and evaporated to dryness in vacuo giving a crude which was purified by flash chromatography (petroleum ether-EtOAc 92.5:7.5) affording 2.97 g of the pure title product.

¹H-NMR (CDCl₃, δ): 1.00-2.10 (m, 17H), 2.20-2.52 (m, 2H), 3.30-3.72 (m, 4H), 4.25-4.45 (m, 2H), 6.90-7.35 (m, 4H)

4-Cyclohexyl-3-(2-fluorophenyl)-4-hydroxybutfraldehyde diethyl acetal upper TLC Rf diastereoisomer (Compound 1c)

4-Cyclohexyl-3-(2-fluorophenyl)-4-hydroxybutyraldehyde diethyl acetal lower TLC Rf diastereoisomer

To a solution of 0.84 g of the compound 1b in 25 ml of MeOH stirred at 0° C. was added 0.095 g of NaBH₄ and the mixture was stirred at r.t. for 5 h. The solvent was evaporated and the reaction crude was taken up with H₂O (15 ml) and extracted with EtOAc (2×15 ml). The organic layer was separated, washed with brine (2×15 ml), dried (Na₂SO₄) and evaporated to dryness in vacuo to give a crude which was purified by flash chromatography (petroleum ether-EtOAc gradient from 92:8 to 85:15) afforded compound 1c (upper Rf) (0.56 g, 63%) and the corresponding compound with lower Rf (4.8%). TLC eluent petroleum ether-EtOAc 9:1.

1c: ¹H-NMR (CDCl₃, δ): 0.90-1.35 (m, 12H), 1.50-1.95 (m, 5H and OH), 2.00-2.10 (m, 2H), 3.25-3.75 (m, 6H), 4.25 (t, 1H), 6.95-7.30 (m, 3H), 7.40-7.55 (m, 1H)

4-Cyclohexyl-3-(2-fluorophenyl)-4-methoxybutiraldehyde diethyl acetal (Compound 1d)

To a solution of 0.514 g of compound 1c in 2 ml of anhydrous DMF stirred at r.t. was added 0.091 g of 60% NaH. The reaction mixture was stirred at r.t. for 1 h, then 0.142 ml of methyl iodide was added and the resulting mixture was stirred at r.t. for 2 h. Afterwards, the reaction mixture was poured into water (30 ml), extracted with 2×20 ml of EtOAc, which was washed, dried (Na₂SO₄) and evaporated to dryness in vacuo affording 0.50 g of the title compound as a crude, which could be used in the following step without further purification.

¹H-NMR (CDC₁3, δ): 0.90-1.40 (m, 12H), 1.50-1.90 (m, 5H), 1.92-2.20 (m, 2H), 3.05 (t, 1H), 3.20 (s, 3H), 3.20-3.70 (m, 5H), 4.05-4.18 (m, 1H), 6.90-7.20 (m, 3H), 7.40-7.55 (m, 1H)

4-Cyclohexyl-3-(2-fluorophenyl)-4-methoxybutiraldehyde (Compound 1e)

A mixture of 0.502 g of the compound 1d, 3.5 ml of 50% aq. trifluoroacetic acid and 7 ml of CH₂Cl₂ was stirred for 2 h at r.t., and then diluted with 8 ml of CH₂Cl₂. The organic layer was separated, washed with brine (2×15 ml), dried (Na₂SO₄) and evaporated to dryness in vacuo to afford a crude (0.365 g), used in the next step without further purification.

¹H-NMR (CDCl₃, δ): 0.95-1.40 (m, 6H), 1.41-2.00 (m, 5H), 2.65-2.95 (m, 2H), 3.05-3.15 (m, 1H), 3.35, 3.37(2s, 3H), 3.70-3.90 (m, 1H), 6.90-7.25 (m, 3H), 7.40-7.55 (m, 1H), 9.65 (s, 1H)

1-[4-Cyclohexyl-3-(2-fluoro-phenyl)-4-methoxybutyl]-4-[2-(2,2,2-trifluoroethoxy)phenyl]piperazine

A mixture of 0.212 g of the compound 1e, 0.237 g of 1-[2-(2,2,2-trifluoroethoxy)phenyl]piperazineHCl, 0.24 g of sodium triacetoxyborohydride, 0.11 ml of AcOH and 6 ml of CH₂Cl₂ was stirred at r.t. for 1 h, kept overnight resting, alkalinised with 20% aq. Na₂CO₃. The organic layer was separated, washed with brine (2×30 ml), dried (Na₂SO₄) and evaporated to dryness in vacuo the give a crude (0.46 g), which was purified by flash chromatography (petroleum ether—EtOAc 7:3) affording the title compound (0.25 g; 62%).

¹H-NMR (CDCl₃, δ): 0.95-1.30 (m, 6H), 1.55-2.50 (m, 9H), 2.45-270 (m, 4H), 3.00-3.20 (m, 5H), 3.20-3.38 (m, 4H), 435 (q, 2H), 6.85-7.20 (m, 7H), 7.40-7.55 (m, 1H)

### EXAMPLE 2

### 1-(4-Fluoro-2-methoxyphenyl)-4-[4-oxo-3-(2-trifluoromethoxyphenyl)pentyl]piperazine

1-(2-Trifluoromethoxyphenyl)propan-2-one (Compound 2a)

A solution of 1.9 g of 1-(2-trifluoromethoxy)benzaldehyde, 4 ml of EtOH, 1.3 ml of 96% 2-nitroethane and 0.10 ml of n-butylamine was stirred at reflux for 18 h. Afterwards, it was diluted with H₂O, extracted with EtOAc (2×30 ml), washed with H₂O (2×30 ml), brine, dried (Na₂SO₄) ed evaporated in vacuo to afford 2.47 g of an orange oil, which was purified by flash chromatography (PE-EtOAc 100:5). Evaporation of the collected fractions yielded 1.60 g of 2-nitro-3-(2-trifluoromethoxyphenyl)prop-2-ene as a pale yellow oil.

¹H-NMR (CDCl₃, δ): 2.35 (s, 3H), 7.30-7.55 (m, 4H), 8.10 (s,1H)

A mixture of 1.6 g of the above compound, 0.024 g of Fe(ClO₄)₃, 3.0 g of Fe, 6 ml of H₂O was heated at reflux and stirred for 7.5 h. After overnight resting at r.t., was added 2.80 ml of 37% HCl, heating for 1 h. After cooling, the mixture was extracted with EtOAc (3×40 ml), which was dried (Na₂SO₄) ed evaporated in vacuo to give the title compound (g 1.28) as an orange oil.

¹H-NMR (CDCl₃, δ): 2.22 (s, 3H), 3.77 (s, 2H), 7.15-7.40 (m, 4H)

4-Oxo-3-(2-trifluoromethoxyphenyl)pentanal diethyl acetal (Compound 2b)

To a suspension of 1.87 g of 60% NaH oil dispersion in 10 ml of anhydrous DMF was added dropwise during 6 min under a nitrogen stream, a solution of compound 2a in 15 ml of DMF and the reaction mixture was stirred at r.t. for 3 h. After overnight resting, was added 0.447 g of 2-bromoacetaldehyde diethyl acetal (97%) in 5 ml of DMF; the mixture was stirred at r.t. for 30′, then at 80° C. for 3 h. Afterwards, the mixture was diluted with H₂O (250 ml), acidified with HCl 2N, extracted with Et₂O (3×50 ml), washed with H₂O (40 ml), dried (Na₂SO₄) and evaporated in vacuo, affording a crude (brownish oil), which was purified by flash chromatography (PE-EtOAc 100:2) to yield 1.44 g of compound 2b as a yellowish oil.

¹H-NMR (CDCl₃, δ): 1.08-1.32 (m, 6H), 1.75-1.95 (m, 1H), 2.08 (s, 3H), 2.35-2.60 (m, 1H), 3.20-3.80 (m, 4H), 4.20-4.40 (2H), 7.15-7.35 (4H)

4-Oxo-3-(2-trifluoromethoxyhenyl)pentanal (Compound 2c)

The title compound was obtained following the procedure described for Compound 1e but using as a starting material Compound 2b instead of Compound 1d. After the usual work-up procedure, the title compound was obtained (99%) and used without further purification in the next step.

¹H-NMR (CDCl₃, δ): 2.12 (s, 3H), 2.58 (dd, 1H), 3.40 (dd, 1H), 4.61 (dd, 1H), 7.11-7.40 (m, 4H), 9.75 (s, 1H)

1-(4-Fluoro-2-methoxyphenyl)-4-[4-Oxo-3-(2-trifluoromethoxyphenyl)pentyl]piperazine

The title compound was obtained following the procedure described for the Compound of Example 1, but using as a starting material Compound 2c instead of compound 1e and 1-(4-fluoro-2-methoxyphenyl)piperazine instead of 1-(2,2,2-trifluoroethoxyphenyl)piperazine. Purification by flash chromatography (PE-EtOAc 7:3) yielded the title compound (60%). Oil.

¹H-NMR (CDCl₃, δ): 1.65-1.85 (m, 1H), 2.10 (s, 3H), 2.25-2.45 (m, 3H), 2.50-2.70 (m, 4H), 2.85-3.10 (m, 4H), 3.82 (s, 3H), 4.15-4.31 (m, 1H), 6.50-6.68 (m, 2H), 6.78-6.90 (m, 1H), 7.20-7.35 (m, 4H)

### EXAMPLE 3

### 1-(4-Fluoro-2-methoxyphenyl)-4-[4-hydroxy-3-(2-trifluoromethoxyphenyl)pentyl]piperazine

The title compound was synthesised using the method described for compound 1c but starting from the Compound of Example 2 instead of Compound 1b. After the usual work-up procedure, the tile compound was isolated (93.1%) and characterized by LC as a mixture of diastereomers (RS;SR-RS, RS 78.8:20.5). LC purity: 98.6%

¹H-NMR (CDCl₃, δ): 0.95; 1.07 (2d, 3H), 1.80-2.10 (m, 2H), 2.35-2.50 (m, 2H), 2.60-2.85 (m, 4H), 2.92-3.18 (m, 4H), 3.18-3.35 (m, 1H), 3.80 (s, 3H), 4.00-4.20 (m, 1H), 4.60-6.10 (b, 1H), 6.50-6.70 (m, 2H), 6.78-6.95 (m, 1H), 7.15-7.35 (m, 3H), 7.60-7.75 (m, 1H)

### EXAMPLE 4

### 1-[5-(2,3-Dihydro-1,4-benzodioxinyl)]-4-[4-oxo-3-(2-trifluoromethoxyphenyl)pentyl]piperazine

The title compound was obtained following the procedure described for for the Compound of Example 1, but using as a starting material Compound 2c instead of Compound 1e and 1-(2,3-dihydro-1,4-benzodioxin-5-yl)piperazine instead of 1-(2,2,2-trifluoroethoxyphenyl)piperazine. Purification by flash chromatography (PE-EtOAc 7:3) yielded the title compound (33%). Oil.

¹H-NMR (CDCl₃, δ): 1.65-1.85 (m, 1H), 2.10 (s, 3H), 2.25-2.40 (m, 3H), 2.50-2.70 (m, 4H), 2.90-3.15 (m, 4H), 4.18-4.40 (m, 4H), 6.48-6.65 (m, 2H), 6.70-6.82 (m, 1H), 7.20-7.35 (m, 4H)

### EXAMPLE 5

### 1-[5-(2,3-Dihydro-1,4-benzodioxinyl)]-4-[4-hydroxy-3-(2-trifluoromethoxyphenyl)pentyl]piperazine

The title compound was synthesised using the method described for Compound 1c but starting from the compound of Example 4 instead of Compound 1b. After the usual work-up procedure, the title compound was isolated (92.7%) and characterized by LC as a mixture of diastereomers (RS,SR-RS,RS 72.9:19.4). LC Purity: 92.3%

¹H-NMR (CDCl₃, δ): 0.95; 1.07 (2d, 3H), 1.80 2.15 (m, 2H), 2.30-2.50 (m, 2H), 2.60-2.85 (m, 4H), 3.00-3.20 (m, 4H), 3.20-3.40 (m, 1H), 4.00-4.15 (m, 1H), 4.15-4.40 (m, 4H), 4.60-6.20 (b, 1H), 6.45-6.65 (m, 2H), 6.65-6.85 (m, 1H), 7.15-7.30 (m, 3H), 7.60-7.75 (m, 1H)

### EXAMPLE 6

### 1-[5-(2,3-Dihydro-1,4-benzodioxinyl)]-4-[4-hydroxy-3-(2-trifluoromethoxyphenyl)hexyl]piperazine

2-Allyl-2-(2-trifluoromethoxyphenyl)acetonitrile (Compound 6a)

To 2.74 ml of a 2M solution of LDA in THF cooled at −78° C. was added dropwise 2-(2-trifluoromethoxyphenyl)acetonitrile in 20 ml of THF; the mixture was stirred at the same temperature for 10 min. Afterwards, was added a mixture of 0.474 ml of allyl bromide and 0.446 g of HMPTA and the reaction was stirred at −78° C. for 2 h, then it was brought to r.t. by spontaneous heating. After overnight resting, it was quenched with an aq. saturated solution of NH₄Cl and extracted with EtOAc. The combined extracts were dried (Na₂SO₄) and evaporated to dryness. The crude was purified by flash chromatography (PE-EtOAc 95:5) affording the title product as a pale yellow oil (1.015 g).

¹H-NMR (CDCl₃, δ): 2.60 (t, 2H), 4.15-4.26 (m, 1H), 5.16-5.29 (m, 2H), 5.79-5.91 (m, 1H), 7.28-7.41 (m, 3H), 7.51-7.67 (m, 1H).

2-(2-Trifluoromethoxyphenyl)pent-4-enal (Compound 6b)

To a solution of 0.88 g of Compound 6a in anhydrous toluene (50 ml) was added dropwise at r.t. 4.01 ml of DIBAL-H (2M sol. in toluene) over 10 min. The reaction mixture was stirred at r.t. for 2 h, diluted with 0.01 N HCl, extracted with EtOAc (2×50 ml); the combined extracts were washed with H₂O, dried (Na₂SO₄) and evaporated to dryness in vacuo. Compound 6b was obtained as a pale yellow oil and used in the next step without further purification.

¹H-NMR (CDCl₃, δ): 2.49-2.68 (m, 1H), 2.80-2.95 (m, 1H), 4.02-4.15 (m, 1H), 5.12-5.26 (m, 2H), 5.75-5.85 (m, 1H), 7.28-7.37 (m, 4H), 9.75 (bs, 1H)

2-[1-[3-Butenyl-1-(2-trifluoromethoxyphenyl)]]-1,3-dioxolane (Compound 6c)

A solution of 0.72 g of Compound 6b, 0.052 g of p-toluenesulfonic acid monohydrate, 0.328 ml of ethylen glycol in 30 ml of toluene was stirred at reflux for 8 h. Afterwards, the solvent was removed by evaporation in vacuo, diluted with EtOAc and aq. NaHCO₃; the organic layer was separated, dried on Na₂SO₄ and evaporated to dryness in vacuo. The crude was purified by flash chromatography (PE-EtOAc 95:5) affording the title product as a pale yellow oil (0.85 g).

¹H-NMR (CDCl₃, δ): 2.39-2.51 (m, 1H), 2.52-2.79 (m, 1H), 3.46-3.57 (m, 1H), 3.80-3.92 (m, 4H), 4.88-4.95 (m, 2H), 4.96-5.12 (m, 1H), 5.72-5.81 (m, 1H), 7.21-7.33 (m, 3H), 7.33-7.45 (m, 1H).

3-[1,3-Dioxolan-2-yl]-3-(2-trifluoromethoxyphenyl)]propionaldehyde (Compound 6d)

To a biphasic mixture of 0.31 g of Compound 6c, 10 ml of Et₂O and 10 ml of H₂O vigorously stirred was added 0.196 ml of OSO₄ followed by addition of 3.6 g of NaIO₄ in aliquots over a period of 20 min. After 6 h, the organic layer was separated, the aqueous layer was extracted with Et₂O. the combined organic layers were dried (Na₂SO₄) and evaporated to dryness in vacuo. The crude was purified by flash chromatography (PE-EtOAc 8) to afford 0.311 g of the title product.

¹H-NMR (CDCl₃, δ): 2.52-2.69 (m, 1H), 2.88-3.03 (m, 1H), 3.81-3.93 (m, 4H), 3.94-4.15 (m, 1H), 5.03-5.08 (m, 1H), 7.22-7.38 (m, 3H), 7.39-7-55 (m, 1H), 9.76 (bs, 1H).

1-(2,3-Dihydro-1,4-benzodioxin-5-yl)-4-[3-(1,3-dioxolan-2-yl)-3-(2-trifluoromethoxyphenyl)propyl]piperazine (Compound 6e)

The title compound was obtained following the procedure described for the compound of Example 1, but using as a starting material Compound 6d instead of Compound 1e and 1-(2,3-dihydro-1,4-benzodioxin-5-yl)piperazine instead of 1-(2,2,2-trifluoroethoxyphenyl)piperazine. Purification by flash chromatography (PE-EtOAc 1:1) yielded the title compound (61%). Oil.

¹H-NMR (CDCl₃, δ): 1.80-2.01 (m, 1H), 2.02-2.44 (m, 3H), 2.45-2.71 (m, 4H), 2.92-3.07 (m 4H), 3.41-3.61 (m, 1H), 3.81-3.93 (m, 4H), 4.21-4.39 (m, 4H), 5.01-5.05 (m, 1H), 6.49-6.57 (m, 2H), 6.71-6.82 (m, 1H), 7.21-7.39 (m, 3H), 7.41-7-58 (m, 1H).

1-(2,3-Dihydro-1,4-benzodioxin-5-yl)-4-[3-formyl-3-(2-trifluoromethoxyphenyl)propyl]piperazine (Compound 6f)

A mixture of 0.12 g of Compound 6e, 0.005 g of 4-toluenesulfonic acid monohydrate, 1 ml of H₂O and 7 ml of dioxane was stirred at reflux for 24 h. Afterwards, the solvent was removed by evaporation in vacuo, the residue was diluted with EtOAc and aq. NaOH; the organic layer was separated, dried on Na₂SO₄ and evaporated to dryness in vacuo. The crude, obtained as a pale yellow oil, was used in the next step without further purification.

¹H-NMR (CDCl₃, δ): 1.79-1.99 (m, 1H), 2.25-2.49 (m, 3H), 2.50-2.71 (m, 4H), 2.91-3.12 (m 4H), 4.07-4.15 (m, 1H), 4.16-4.39 (m, 4H), 6.48-6.61 (m, 2H), 6.73-6.86 (m, 1H), 7.20-7.38 (m, 3H), 9.81 (bs, 1H).

4,4-Diethoxy-2-(2-trifluoromethoxyphenyl)butyronitrile (Compound 6g)

The title compound was synthesised following the procedure reported for Compound 6a but using 2-bromoacetaldehyde diethyl acetal instead of allyl bromide. After spontaneous heating to r.t. during 2 hours, the reaction misture was refluxed for additional 2 h. After the usual work-up procedure, the crude was purified by flash chromatography (PE-EtOAc 95:5) affording the title product (47.7%) as a pale yellow oil.

¹H-NMR (CDCl₃, δ): 1.12-1.35 (m, 6H), 2.07-2.27 (m, 2H), 3.50-3.71 (m, 4H), 4.22-4.38 (m 1H), 4.65-4.71 (m, 1H), 7.19-7.48 (m, 3H), 7.49-7.63 (m, 1H).

4-Oxo-2-(2-trifluoromethoxyphenyl)butyronitrile (Compound 6h)

The title compound was obtained following the procedure described for Compound 1e but using as a starting material Compound 6g instead of Compound 1d. After the usual work-up procedure the so obtained title compound was used without further purification in the next step.

¹H-NMR (CDCl₃, δ): 2.98-3.31 (m, 2H), 4.65-4.78 (m 1H), 4.65-4.71 (m, 1H), 7.22-7.49 (m, 3H), 7.51-7.66 (m, 1H), 9.81 (bs, 1H).

1-[3-Cyano-3-(2-trifluoromethoxyphenyl)propyl]-4-(2,3-dihydrobenzo-1,4-dioxin-5-yl)piperazine (Compound 6i)

The title compound was obtained following the procedure described for the compound of Example 1 but using as a starting material Compound 6h instead of compound 1e and 1-(2,3-dihydro-1,4-benzodioxin-5-yl)piperazine instead of 1-(2,2,2-trifluoroethoxyphenyl)piperazine. Purification by flash chromatography (PE-EtOAc 1:1) yielded the title compound (93%). Oil.

¹H-NMR (CDCl₃, δ): 1.99-2.14 (m, 1H), 2.49-2.71 (m, 3H), 3.01-3.19 (m, 4H), 4.20-4.38 (m 4H), 4.40-4.55 (m, 1H), 6.49-6.65 (m, 3H), 6.72-6.88 (m, 1H), 7.24-7.41 (m, 3H), 7.52-7.68 (m, 1H).

1-(2,3-Dihydro-1,4-benzodioxin-5-yl)-4-[3-formyl-3-(2-trifluoromethoxyphenyl)propyl]piperazine (Compound 6f)

To a solution of 0.414 g of Compound 6i in anhydrous CH₂Cl₂ (50 ml) was added dropwise at −78° C. 1.2 ml of 1 M DIBAL-H in toluene. The reaction was allowed to warm up to and stirred overnight; afterwards, it was diluted with water, extracted with CH₂Cl₂ (2×50 ml); the combined extracts were washed with H₂O, dried (Na₂SO₄) and evaporated to dryness in vacuo. Purification by flash chromatography (CH₂Cl₂-MeOH 95:5) yielded the title compound (0.23 g; 55.3%). Oil.

1-[5-(1,4-benzodioxinyl)]-4-[4-hydroxy-3-(2-trifluoromethoxyphenyl)hexyl]piperazine

Into a solution of 0.1 g of Compound 6f in 10 ml of THF cooled at 0-5° C. was dropped a 1M solution of ethylmagnesium bromide in THF (0.888 ml). The reaction mixture was allowed to warm up to r.t. and stirred at the same temperature for 3 h. Afterwards, it was quenched with an aq. saturated solution of NH₄Cl, alkalinised and extracted with EtOAc. The combined extracts were dried (Na₂SO₄) and evaporated to dryness. The crude was purified by flash chromatography (CH₂Cl₂-MeOH/NH₃ 97:3) affording the title product as a yellow glassy oil (84.4%).

¹H-NMR (CDCl₃, δ): 0.86-0.99 (m, 3H), 1.21-1.35 (m, 2H), 1.36-1.65 (m, 1H), 1.66-1.89 (m, 1H) 1.90-2.21 (m, 2H), 2.25-2.95 (m, 6H), 2.96-3.27 (m 4H), 3.61-3.80 (m, 1H), 4.21-4.41 (m, 4H), 6.49-6.61 (m, 2H), 6.65-6.86 (m, 1H), 7.15-7.39 (m, 4H).

[M+H]⁺=481.6

### EXAMPLE 7

### 1-[5-(2,3-Dihydro-1,4-benzodioxinyl)]-4-[4-hydroxy-3-(2-trifluoromethoxyphenyl)hex-5-enyl]piperazine

The title product was obtained by the same procedure described for the compound of Example 6 but using Compound 6f and vinylmagnesium bromide (1M in THF) instead of ethyl magnesium bromide in THF. The crude was purified by flash chromatography (CH₂Cl₂-MeOH/NH₃ 95:5) affording the title product as a yellow glassy oil (42.6%).

¹H-NMR (CDCl₃, δ): 1.76-1.98 (m, 1H) 1.99-2.28 (m, 1H), 2.29-2.51 (m, 2H), 2.52-2.89 (m 4H), 2.89-3.25 (m, 6H), 4.20-4.43 (m, 4H), 4.65-5.31 (m, 3H), 5.61-5.70 (m, 1H), 6.49-6.62 (m, 2H), 6.70-6.89 (m, 1H), 7.15-7.42 (m, 4H).

[M+H]⁺=479.5

### EXAMPLE 8

### 1-[5-(2,3-Dihydro-1,4-benzodioxinyl)]-4-[4-hydroxy-5-methyl-3-(2-trifluoromethoxyphenyl)hexyl]piperazine

The title product was obtained by the same procedure described for the compound of Example 6 but using Compound 6f and isopropylmagnesium chloride(2 M in THF) instead of ethylmagnesium bromide in THF. The crude was purified by flash chromatography (CH₂Cl₂-MeOH/NH₃ 97:3) affording the title product as a yellow glassy oil (30.9%).

¹H-NMR (CDCl₃, δ): 0.78-0.98 (m, 6H) 1.15-1.45 (m, 2H), 1.71-1.91 (m, 2H), 1.92-2.19 (m, 1H), 2.25-2.51 (m 2H), 2.52-2.95 (m, 4H), 3.01-3.29 (m, 5H), 3.51-3.72 (m, 1H), 4.19-4.40 (m, 4H), 6.47-6.63 (m, 2H), 6.67-6.87 (m, 1H), 7.15-7.41 (m, 4H).

[M+H]⁺=495.6

### EXAMPLE 9

### 1-[5-(2,3-Dihydro-1,4-benzodioxinyl)]-4-[4-methoxy-3-(2-trifluoromethoxyphenyl)-5-hexenyl]piperazine

4,4-Diethoxy-2-(2-trifluoromethoxyphenyl)butyraldehyde (Compound 9a)

The title compound was prepared following the procedure described for Compound 6f (alternative method) but starting from Compound 6g instead of Compound 6i. The crude was purified by flash chromatography (CH₂Cl₂-MeOH 99:1) affording the title product as a yellow glassy oil (41.9%).

¹H-NMR (CDCl₃, δ): 1.11-1.34 (m, 6H); 1.85-2.04 (m, 1H); 2.41-2.62 (m, 1H); 3.34-3.77 (m, 4H); 4.08-4.19 (m, 1H); 4.39-4.51 (m, 1H); 7.19-7.42 (m, 4H); 9.66 (s, 1H).

6,6-Diethoxy-4-(2-trifluoromethoxyphenyl)hex-1-en-3-ol (Compound 9b)

The title product was obtained by the same procedure described for the compound of Example 6 but using vinylmagnesium bromide (1M in THF) instead of ethyl magnesium bromide in THF and starting from Compound 9a. The crude was purified by flash chromatography (PE-EtOAc 4:6). Yield: 63.1%.

¹H-NMR (CDCl₃, δ): 1.04-1.32 (m, 6H); 1.93-2.09 (m, 1H); 2.01-2.39 (m, 1H); 2.51 (bs, 1H); 3.28-3.75 (m; 5H); 4.18-4.36 (m, 2H); 5.01-5.22 (m, 2H); 5.67-5.87 (m, 1H); 7.14-7.43 (m, 4H).

4-Methoxy-3-(2-trifluoromethoxyphenyl)but-3-enal diethyl acetal (Compound 9c)

The title compound was synthesised as described for Compound 1d using as a starting material Compound 9b instead of Compound 1c.The crude was used in the next step without further purification.

¹H-NMR (CDCl₃, δ): 1.01-1.42 (m, 6H); 1.86-2.04 (m, 1H); 2.24-2.43 (m, 1H); 3.24 (s, 3H); 3.30-3.79 (m, 6H); 4.13-4.28 (m, 1H); 4.98-5.17 (m, 2H); 5.50-5.71 (m, 1H); 7.13-7.29 (m, 3H); 7.31-7.48 (m, 1H).

4-Methoxy-3-(2-trifluoromethoxyphenyl)but-3-enal (Compound 9d)

The title compound was obtained following the procedure described for Compound 1e but using as a starting material Compound 9c instead of compound 1d. After the usual work-up procedure the so obtained title compound was used without further purification in the next step.

¹H-NMR (CDCl₃, δ): 2.58-2.76 (m, 1H); 2.81-3.06 (m, 1H); 3.24 (s, 3H); 3.59-3.72 (m, 1H); 3.73-3.91 (m, 1H); 4.99-5.17 (m, 2H); 5.43-5.67 (m, 1H); 7.13-7.41 (m, 4H); 9.61-9.69 (m, 1H).

1-[5-(2,3-Dihydro-1,4-benzodioxinyl)]-4-[4-methoxy-3-(2-trifluoromethoxyphenyl)-5-hexenyl]piperazine

The title compound was prepared using the method described for the Compound of Example 1 but using compound 9d instead of Compound 1e and 1-(2,3-dihydro-1,4-benzodioxin-5-yl)piperazine instead of 1-(2,2,2-trifluoroethoxyphenyl)piperazine. The yield after flash chromatography (PE-acetone 6:4) was 34.5%.

¹H-NMR (CDCl₃, δ): 1.74-1.93 (m, 1H); 2.09-2.42 (m, 3H); 2.48-2.71 (m, 4H); 2.89-3.28 (m, 4H); 3.18-3.31 (m, 4H); 3.54-3.68 (m, 1H); 4.17-4.38 (m, 4H); 4.96-5.30 (m, 2H); 5.47-5.68 (m, 1H); 6.47-6.63 (m, 2H); 6.71-6.85 (m, 1H); 7.12-7.31 (m, 3H); 7.3-7.48 (m, 1H).

### EXAMPLE 10

### 1-[5-(2,3-Dihydro-1,4-benzodioxinyl)]-4-[(4-methoxy-3-phenyl)heptyl]piperazine

4-Oxo-3-phenylheptanal diethyl acetal (Compound 10a)

The title compound was prepared following the method described for Compound 2b but using 1-phenyl-2-pentanone instead of Compound 2a. The crude was purified by flash chromatography (EtOAc-PE 95:5). Yield: 59.2%.

¹H-NMR (CDCl₃, δ): 0.78-0.88 (m, 3H), 1.10-1.31 (m, 8H), 1.42-1.72 (m, 2H), 2.38-2.50 (m, 3H), 3.31-3.90 (m, 4H), 4.18-4.35 (m, 1H), 7.05-7.42 (m, 5H).

4-Hydroxy-3-phenylheptanal diethyl acetal (Compound 10b)

The title compound was obtained following the procedure described for Compound 1c but using as a starting material Compound 10a instead of Compound 1b. After the usual work-up procedure, the crude was purified by flash chromatography (EtOAc-PE 2:8). Yield: 73.3%.

¹H-NMR (CDCl₃, δ): 0.70-0.82 (m, 3H), 0.90-1.48 (m, 12H), 2.10-2.57 (m, 2H), 3.32-3.94 (m, 4H), 4.08-4.30 (m, 1H), 5.18-5.35 (m, 1H), 7.05-7.42 (m, 5H).

4-Methoxy-3-phenylheptanal diethyl acetal (Compound 10c)

The title compound was obtained following the procedure described for Compound 1d, but using as a starting material Compound 10b instead of Compound 1c. The title product was used in the next step without further purification.

¹H-NMR (CDCl₃, δ): 0.70-0.82 (m, 3H), 1.02-1.48 (m, 12H), 2.02-2.15 (m, 1H), 2.95-3.01 (m, 1H), 3.20-3.80 (m, 7H), 4.15-4.35 (m, 1H), 7.05-7.42 (m, 5H).

4-Methoxy-3-phenylheptanal (Compound 10d)

The title compound was obtained following the procedure described for Compound 1e, but using as a starting material Compound 10c instead of compound 1c. The title product was used in the next step without further purification.

¹H-NMR (CDCl₃, δ): 0.70-1.59 (m, 7H), 2.81-2.95 (m, 2H), 3.22-3.61 (m, 5H), 7.05-7.42 (m, 5H). 9.75 (s, 1H)

1-[5-(2,3-Dihydro-1,4-benzodioxinyl)]-4-[(4-methoxy-3-phenyl)heptyl]piperazine

The title compound was prepared using the method described for the Compound of Example 1 but using compound 10d instead of Compound 1e and 1-(2,3-dihydro-1,4-benzodioxin-5-yl)piperazine instead of 1-(2,2,2-trifluoroethoxyphenyl)piperazine. The yield after flash chromatography (EtOAc-PE-MeOH/NH₃ 1:1:0.2 to 8:2:0.2) was 39.5%.

¹H-NMR (CDCl₃, δ): 0.78-0.91 (m, 3H), 1.20-1.48 (m, 4H),1.90-2.07 (m, 2H), 2.19-2.33 (m, 2H), 2.50-2.68 (m, 4H), 2.80-2.91 (m, 1H), 2.99-3.12 (m, 4H), 3.18-3.30 (1H), 3.35 (s, 3H), 4.20-4.38 (m, 4H), 6.48-6.62 (m, 2H), 6.78 (s, 1H), 7.25-7.33 (m, 5H).

### EXAMPLE 11

### 1-[5-(2,3-Dihydro-1,4-benzodioxinyl)]-4-[(4-methoxy-3-phenyl)pentyl]piperazine

4-Oxo-3-phenylpentanal diethyl acetal (Compound 11a)

The title compound was prepared using the method described for Compound 2b but using 1-phenylacetone (commercially available) instead of Compound 2a. The crude was used in the next step without further purification. Yield: 93%.

¹H-NMR (CDCl3, _{δ}): 1.10-1.25 (m, 6H), 1.82-2.17 (m, 4H), 2.32-2.50 (m, 1H), 3.30-3.70 (m, 4H), 3.82 (t, 1H), 4.23-4.33 (m, 1H), 7.15-7.39 (m, 5H).

4-Hydroxy-3-phenylpentanal diethyl acetal (Compound 11b)

The title compound was obtained following the procedure described for Compound 1c but using as a starting material Compound 11a instead of Compound 1b. After the usual work-up procedure, the crude was used in the next step without further purification. Yield: 60%.

¹H-NMR (CDCl₃, δ): 1.00-1.32 (m, 9H), 2.05-2.15 (m, 1H),1.89-2.17 (m, 2H), 2.68-2.81 (m, 1H), 3.28-3.71 (m, 4H), 3.82-4.02 (m, 1H), 4.15-4.26 (m, 1H), 7.12-7.41 (m, 5H).

4-Methoxy-3-phenylpentanal diethyl acetal (Compound 11c)

The title compound was obtained following the procedure described for Compound 1d, but using as a starting material Compound 11b instead of compound 1c. The crude was purified by flash chromatography (EtOAc 5-PE 95).

4-Methoxy-3-phenylpentanal (Compound 11d)

The title compound was obtained following the procedure described for Compound 1e, but using as a starting material Compound 11c instead of compound 1d. The title product was used in the next step without further purification.

1-[5-(2,3-Dihydro-1,4-benzodioxinyl)]-4-[(4-methoxy-3-phenyl)pentyl]piperazine

The title compound was prepared using the method described for the Compound of Example 1 but using compound 9d instead of Compound 1e and 1-(2,3-dihydro-1,4-benzodioxin-5-yl)piperazine instead of 1-(2,2,2-trifluoroethoxyphenyl)piperazine. The yield after flash chromatography (EtOAc-PE-MeOH/NH₃ 8:2:0.1 to 8:2:0.3 was 15.5%.

¹H-NMR (CDCl₃, δ): 1.04 (d, 3H), 1.88-2.05 (m, 2H),2.18-2.31 (m, 2H), 2.50-2.68 (m, 4H), 2.73-2.85 (m, 1H), 2.97-3.12 (m, 4H), 3.30 (s, 3H), 3.42-3.50 (m, 1H), 4.18-4.38 (m, 4H), 6.48-6.62 (m, 2H), 6.71-6.82 (t, 1H), 7.15-7.33 (m, 5H).

### EXAMPLE 12

### 1-[5-(2,3-Dihydro-1,4-benzodioxinyl)]-4-[(4-propoxy-3-phenyl)heptyl]piperazine

4-Propoxy-3-phenylheptanal (Compound 12a)

The title compound was obtained following the procedure described for Compound 1d, but using as a starting material Compound 10b instead of compound 1c. The crude was purified by flash chromatography (EtOAc 5-PE 95).

4-Propoxy-3-phenylheptanal (Compound 12b)

The title compound was obtained following the procedure described for Compound 1d, but using as a starting material Compound 12a instead of compound 1c. The crude was used in the next step without further purification.

1-[5-(2,3-Dihydro-1,4-benzodioxinyl)]-4-[(4-methoxy-3-phenyl)heptyl]piperazine

The title compound was prepared using the method described for the Compound of Example 1 but using compound 12b instead of Compound 1e and 1-(2,3-dihydro-1,4-benzodioxin-5-yl)piperazine instead of 1-(2,2,2-trifluoroethoxyphenyl)piperazine. The yield after flash chromatography (EtOAc-PE-MeOH/NH₃ 4:6:0.1 to EtOAc-MeOH/NH₃ 97:3 was 9.5%.

¹H-NMR (CDCl₃, δ): 0.72-0.92 (m, 6H), 1.15-1.61 (m, 6H), 1.89-2.08 (m, 2H), 2.18-2.31 (m, 2H), 2.50-2.68 (m, 4H), 2.78-2.92 (m, 1H), 2.97-3.12 (m, 4H), 3.28-3.43 (m, 3H), 4.18-4.38 (m, 4H), 6.48-6.62 (m, 2H), 6.71-6.82 (t, 1H), 7.15-7.33 (m, 5H).

### EXAMPLE 13

### 1-[3-(2-Cyanophenyl)-4-cyclohexyl-4-oxobutyl]-4-[5-(2,3-dihydro-1,4-benzodioxinyl)]piperazine

2-(2-Cyclohexyl-2-oxoethyl)benzonitrile (Compound 13a)

To a solution of 0.47 g of 2-tolunitrile in 4 ml of THF was added 0.535 ml of 1,3-dimethyl-3,4,5,6-tetrahydro-2(1H)pyrimidinone(DMPU) and the mixture was cooled at −78° C.; 2.22 ml of a 2M sol. of LDA in THF was dropped during 5 min., then the reaction mixture was stirred at the same temperature for 15 min. followed by dropwise addition of 0.757 g of N-methyl-N-methoxycyclohexanecarboxamide in 4 ml of THF. After 1 h stirring at −78° C., the reaction mixture was quenched with a 10% aq. sol. of NH₄Cl. The temperature was allowed to raise at r.t. and the mixture was extracted with EtOAc (2×20 ml), washed with 30 ml of brine, dried on Na₂SO₄ and evaporated to dryness in vacuo. The crude was purified by flash chromatograophy (PE-EtOAc 85:15 to 1:1) to afford 0.34 g of the title compound.

¹H-NMR (CDCl₃, δ): 1.10-2.05 (m, 10H); 2.45-2.602 (m, 1H); 4,00 (m,2H); 7.20-7.43 (m,2H); 7.48-7.70 (m,2H);

3-(2-Cyanophenyl)-4-cyclohexyl-4-oxobutyraldehyde diethyl acetale (Compound 13b)

The title compound was prepared using the method described for Compound 2b but using Compound 13a instead of Compound 2a. The crude was purified by flash chromatography (toluene-EtOAc 97:3). Yield: 39.1%.

¹H-NMR (CDCl3, _{δ}): 1.05-1.90 (m,15H); 1.90-2.05 (m,2H); 2.32-2.60 (m,2H); 3.20-3.70 (m,4H); 4.30 (t,1H); 4.55 (t,1H); 7.30-7.45 (m,2H); 7.55 (dd,1H); 7.68 (dd,1H)

3-(2-Cyanophenyl)-4-cyclohexyl-4-oxobutyraldehyde (Compound 13c)

The title compound was obtained following the procedure described for Compound 1d, but using as a starting material Compound 13b instead of compound 1c. The crude was used in the next step without further purification.

¹H-NMR (CDCl₃, δ): 1.00-1.90 (m,10H); 2.05-2.15 (m,1H);2.35-2.50 (m,1H); 2.70 (dd,1H); 3.45 (dd,1H); 4.85 (dd,1H); 7.25 (dd,1H); 7.30-7.40 (m,1H); 7.50-7.60 (m,1H); 7.75 (dd,1H)

1-[3-(2-Cyanophenyl)-4-cyclohexyl-4-oxobutyl]-4-[5-(2,3-dihydro-1,4-benzodioxinyl)]piperazine

The title compound was prepared using the method described for the Compound of Example 1 but using compound 13c instead of Compound 1e and 1-(2,3-dihydro-1,4-benzodioxin-5-yl)piperazine instead of 1-(2,2,2-trifluoroethoxyphenyl)piperazine. The crude was purified by flash chromatography (PE:EtOAc 6:4) to afford the title compound (79.5%).

¹H-NMR (CDCl₃, δ): 1.10-2.10 (m,11H); 2.20-2.50 (m, 4H); 2.50-2.75 (m,4H); 2.92-3.20 (m,4H); 4.20-4.38 (m,4H); 4.55 (t,1H); 6.48-6.65 (m,2H); 6.70-6.85 (m,1H); 7.30-7.45 (m,2H); 7.45-7.60 (m,1H); 7.65-7.75 (m,1H)

### EXAMPLE 14

### (RS,SR)-1-[3-(2-Cyanophenyl)-4-cyclohexyl-4-hydroxybutyl]-4-[5-(2,3-dihydro-1,4-benzodioxinyl)]piperazine

The title compound was synthesised using the method described for compound 1c but starting from the Compound of Example 14 instead of Compound1b. After the usual work-up procedure, the crude was purified by flash chromatography (PE-EtOAc-NH₃/MeOH 65:35:3) affording the title compound (70.5%).

¹H-NMR (CDCl₃, δ): 0.80-1.40 (m,7H); 1.45-1.80 (m,5H);1.85-2.05 (m,1H); 2.20-2.50 (m,2H); 2.50-2.80 (m,4H); 2.95-3.20 (m,4H); 3.30-3.50 (m,1H); 3.50-3.65 (m,1H); 4.20-4.40 (m,4H); 4.40-5.90 (bs,1H); 6.50-6.67 (m,2H);6.70-6.85 (m,1H); 7.20-7.40 (m,1H); 7.50-7.68 (m,2H); 7.93-8.08 (m,1H)

### EXAMPLE 14a

### (RS)-1-[3-(2-Cyanophenyl)-4-cyclohexyl-4-hydroxybutyl]-4-[5-(2,3-dihydro-1,4-benzodioxinyl)]piperazine (enantiomer at Rt=30.298 min.)

This compound was obtained from the Compound of Example 14 resolving by chiral column chromatography using Chiralpak AD (0.46×25 cm), eluting with n-hexane-EtOH 95:5 (flow =1 ml/min; detector UV 254 nm).

### EXAMPLE 14b

### (SR)-1-[3-(2-Cyanophenyl)-4-cyclohexyl-4-hydroxybutyl]-4-[5-(2,3-dihydro-1,4-benzodioxinyl)]piperazine (enantiomer at Rt=34.834 min)

This compound was obtained from the Compound of Example 14 resolving by chiral column chromatography using Chiralpak AD (0.46×25 cm), eluting with n-hexane-EtOH 95:5 (flow =1 ml/min; detector UV 254 nm).

### EXAMPLE 15

### 1-[3-(2-Cyanophenyl)-4-cyclohexyl-4-oxobutyl]-4-(4-fluoro-2-methoxyphenyl)piperazine

The title compound was prepared using the method described for the Compound of Example 1 but using compound 13c instead of Compound 1e and 1-(4-fluoro-2-methoxyphenyl)piperazine instead of 1-(2,2,2-trifluoroethoxyphenyl)piperazine. The crude was purified by flash chromatography (PE:EtOAc 6:4) to afford the title compound (82%).

¹H-NMR (CDCl₃) δ: 1.00-2.10 (m,11H); 2.15-2.75 (m,8H); 2.75-3.15 (m,4H); 3.90 (s,3H); 4.55 (t,1H); 6.50-6.70 (m,2H); 6.80-6.95 (m,1H); 7.30-7.42 (m,2H); 7.45-7.60 (m,1H);7.60-7.72 (m,1H)

### EXAMPLE 16

### 1-[3-(2-Cyanophenyl)-4-cyclohexyl-4-hydroxybutyl]-4-(4-fluoro-2-methoxyphenyl)piperazine

The tiltle compound was synthesised using the method described for Compound 1c but starting from the Compound of Example 14 instead of Compound1b. After the usual work-up procedure, the crude was purified by flash chromatography (PE-EtOAc-NH₃/MeOH 65:35:3) affording the title compound (51.3%).

¹H-NMR (CDCl₃, δ): 0.90-1.20 (m,6H); 1.40-1.85 (m,5H);1.90-2.10 (m,2H); 2.20-2.45 (m,2H); 2.50-2.85 (m,4H); 2.90-3.15 (m,4H); 3.32-3.50 (m,1H) 3.50-3.65 (m,1H); 3.95 (s,3H); 4.60-5.20 (bs,1H); 6.55-6.68 (m,2H); 6.80-6.92 (m,1H); 7.28-7.36 (m,1H); 7.45-7.68 (m,2H);7.95-8.05 (m,1H)

### EXAMPLE 17

### 1-(4-cyclohexyl-4-methoxy-3-phenylbutyl)-4-[5-(2,3-dihydro-1,4-benzodioxinyl)]piperazine

4,4-Dimethoxy-2-phenylbutyronitrile (Compound 17 a)

The title compound was synthesised following the procedure reported for Compound 6b but using 2-bromoacetaldehyde dimethyl acetal instead of allyl bromide and phenylacetonitrile instead of 2-trifluoromethoxyphenylacetonitrile. After spontaneous heating to r.t. during 2 hours, the reaction mixture was refluxed for additional 2 h. After the usual work-up procedure, the crude was purified by flash chromatography (PE-EtOAc 9:1) affording the title product (72.1%) as a pale yellow oil.

¹H-NMR (CDCl₃, δ): 2.02-2.36 (m, 2H); 3.39 (d, 6H); 3.76-4.01 (m, 1H); 4.41-4.54 (m, 1H); 7.30-7.48 (m, 5H).

4,4-Dimethoxy-2-phenylbutyraldehyde(Compound 17b)

The title compound was obtained following the procedure described for Compound 6f (alternative method) but using as a starting material Compound 17a instead of compound 6i. After the usual work-up procedure the crude was purified by flash chromatography (CH₂Cl₂-EtOAc 95:5) to afford the title compound (73.2%).

¹H-NMR (CDCl₃, δ): 1.83-2.02 (m, 1H); 2.39-2.58 (m, 1H); 3.32 (d, 6H); 3.66-3.81 (m, 1H); 4.23-4.38 (m, 1H); 7.07-7.48 (m, 5H); 9.61-9.70 (m, 1H).

4-Cyclohexyl-4-hydroxy-3-phenylbutyraldehyde dimethyl acetal (Compound 17c)

The title compound was obtained following the procedure described for the compound of Example 6 but using as a starting material Compound 17b instead of compound 6f and cyclohexylmagnesium chloride (2M sol. in THF) instead of ethylmagnesium chloride. The crude was purified by flash chromatography (CH₂Cl₂-Acetone 9:1) affording the title product. (55%).

¹H-NMR (CDCl₃, δ): 0.97-2.09 (m, 12H); 2.27-2.45 (m, 1H); 2.88-3.03 (m, 1H); 3.31 (d, 6H); 3.41-3.53 (m, 1H); 4.11-4.22 (m, 1H); 7.21-7.43 (m, 5H).

The OH signal was not detectable.

4-Cyclohexyl-4-methoxy-3-phenylbutyraldehyde dimethyl acetal (Compound 17d)

The title compound was synthesised as described for Compound 1d using as a starting material Compound 17c instead of Compound 1c. After E₂O extraction, the crude was purified by flash chromatography (PE-EtOAc 8:2) affording the title product. (71.4%).

¹H-NMR (CDCl₃, δ): 0.98-1.37 (m, 6H); 1.49-1.98 8m, 6H); 2.17-2.33 (m, 1H); 2.82-3.02 (m, 2H); 3.21 (dd, 6H); 3.37 (s, 3H); 3.94-4.08 (m, 1H); 7.17-7.39 (m, 5H).

4-Cyclohexyl-4-methoxy -3-phenylbutyraldehyde (Compound 17e)

The title compound was obtained following the procedure described for Compound 1e, but using as a starting material Compound 17d instead of compound 1d. The title product was used in the next step without further purification.

¹H-NMR (CDCl₃, δ): 0.93-1.86 (m, 12H); 2.61-2.79 (m, 2H); 3.01-3.16 (m, 1H); 3.31 (s, 3H); 3.41-3.59 (m, 1H); 7.15-7.39 (m, 5H); 9.53-9.61 (m, 1H).

1-(4-Cyclohexyl-4-methoxy-3-phenylbutyl)-4-[5-(2,3-dihydro-1,4-benzodioxinyl)]piperazine

The title compound was prepared using the method described for the Compound of Example 1 but using compound 17e instead of Compound 1e and 1-(2,3-dihydro-1,4-benzodioxin-5-yl)piperazine instead of 1-(2,2,2-trifluoroethoxyphenyl)piperazine. The crude was purified by flash chromatography (PE:Me₂CO 75:25) to afford the title compound (77.4%).

¹H-NMR (CDCl₃, δ): 0.97-1.31 (m, 6H); 1.48-1.99 (m, 6H); 2.07-2.28 (m, 3H); 2.42-2.67 (m, 4H); 2.71-2.90 (m, 1H); 2.92-3.26 (m, 5H); 3.3 (s, 3H); 4.17-4.38 (m, 4H); 6.45-6.64 (m, 2H); 6.66-6.84 (m, 1H); 7.12-7.34 (m, 5H).

### EXAMPLE 18

### 1-(4-Cyclohexyl-4-methoxy-3-phenylbutyl)-4-(4-fluoro-2-methoxyphenyl)piperazine

The title compound was prepared using the method described for the Compound of Example 1 but using compound 17e instead of Compound 1e and 1-(4-fluoro-2-methoxyphenyl)piperazine instead of 1-(2,2,2-trifluoroethoxyphenyl)piperazine. The crude was purified by flash chromatography (PE:Me₂CO 75:25) to afford the title compound (79.8%).

¹H-NMR (CDCl₃, δ): 0.97-1.37 (m, 6H); 1.46-1.98 (m, 6H); 2.07-2.31 (m, 3H); 2.42-2.71 (m, 4H); 2.74-2.80 (m, 1H); 2.81-3.18 (m, 5H); 3.39 (s, 3H); 3.81 (s, 3H); 6.49-6.68 (m, 2H); 6.77-6.92 (m, 1H); 7.13-7.38 (m, 5H).

### EXAMPLE 19

### 1-(4-Cyclohexyl-4-ethoxy-3-phenylbutyl)-4-[5-(2,3-dihydro-1,4-benzodioxinyl)]piperazine

4-Cyclohexyl-4-ethoxy-3-phenylbutyraldehyde dimethyl acetal (Compound 19a)

The title compound was synthesised as described for Compound 1d using as a starting material Compound 17c instead of Compound 1c and ethyl iodide instead of methyl iodide. After E₂O extraction, the crude was purified by flash chromatography (PE-EtOAc 8:2) affording the title product. (50.7%).

¹H-NMR (CDCl₃, δ): 0.91-1.34 (m, 9H); 1.42-1.99 (m, 6H); 2.14-2.34 (m, 1H); 2.80-2.94 (m, 1H); 3.00-3.11 (m, 1H); 3.22 (d, 6H); 3.41-3.57 (m, 2H); 3.92-4.08 (m, 1H); 7.14-7.35 (m, 5H).

4-Cyclohexyl-4-ethoxy-3-phenylbutyraldehyde (Compound 19b)

The title compound was obtained following the procedure described for Compound 1e, but using as a starting material Compound 19a instead of Compound 1d. The title product was used in the next step without further purification.

¹H-NMR (CDCl₃, δ): 0.91-1.39 (m, 8H); 1.48-1.88 (m, 6H); 2.57-2.89 (m, 2H); 3.08-3.20 (m, 1H); 3.23-3.40 (m, 1H); 3.41-3.61 (m, 2H); 7.13-7.38 (m, 5H); 9.57-9.66 (m, 1H).

1-(4-Cyclohexyl-4-ethoxy-3-phenylbutyl)-4-[5-(2,3-dihydro-1,4-benzodioxinyl)]piperazine

The title compound was prepared using the method described for the Compound of Example 1 but using compound 19b instead of Compound 1e and 1-(2,3-dihydro-1,4-benzodioxin-5-yl)piperazine instead of 1-(2,2,2-trifluoroethoxyphenyl)piperazine. The crude was purified by flash chromatography (PE:Me₂CO 8:2) to afford the title compound (60.6%).

¹H-NMR (CDCl₃, δ): 0.97-1.32 (m, 9H); 1.42-1.99 (m, 6H); 2.04-2.36 (m, 3H); 2.46-2.69 (m, 4H); 2.71-2.90 (m, 1H); 2.94-3.21 (m, 5H); 3.26-3.61 (m, 2H); 4.17-4.39 (m, 4H); 6.48-6.74 (m, 2H); 6.68-6.83 (m, 1H); 7.14-7.37 (m, 5H).

### EXAMPLE 20

### 1-(4-Cyclohexyl-4-ethoxy-3-phenylbutyl)-4-(4-fluoro-2-methoxyphenyl)piperazine

The title compound was prepared using the method described for the Compound of Example 1 but using compound 19b instead of Compound 1e and 1-(4-fluoro-2-methoxyphenyl)piperazine instead of 1-(2,2,2-trifluoroethoxyphenyl)piperazine. The crude was purified by flash chromatography (PE:Me₂CO 75:25) to afford the title compound (73%).

¹H-NMR (CDCl₃, δ): 0.96-1.40 (m, 9H); 1.44-1.99 (m, 6H); 2.05-2.34 (m, 3H); 2.42-2.69 (m, 4H); 2.74-2.90 (m, 1H); 2.92-3.16 (m, 5H); 3.21-3.60 (m, 2H); 4.83 (s, 3H); 6.52-6.68 (m,2H); 6.78-6.93 (m, 1H); 7.12-7.36 (m, 5H).

### EXAMPLE 21

### 1-(4-Allyloxy-4-cyclohexyl-3-phenylbutyl)-4-[5-(2,3-dihydro-1,4-benzodioxinyl)]piperazine

4-Allyloxy-4-cyclohexyl-3-phenylbutyraldehyde dimethyl acetal (Compound 21a)

The title compound was synthesised as described for Compound 1d using as a starting material Compound 17c instead of Compound 1c and allyl bromide instead of methyl iodide. The reaction mixture was stirred for 8 h at r.t. and 5 h at 45° C. Et₂O extraction and purification by column chromatography (PE-EtOAc 85:15) yielded the title compound (48.5%).

¹H-NMR (CDCl₃, δ): 0.94-1.38 (m, 6H); 1.51-2.01 (m, 6H); 2.16-2.34 (m, 1H); 2.82-3.01 (m, 1H;) 3.07-3.19 (m, 1H); 3.21 (d, 6H); 3.72-3.88 (m, 1H); 3.90-4.07 (m, 2H); 5.04-5.32 (m, 2H); 5.77-6.00 (m, 1H); 7.16-7.37 (m, 5H).

4-Allyloxy-4-cyclohexyl-3-phenylbutyraldehyde (Compound 21b)

The title compound was obtained following the procedure described for Compound 1e, but using as a starting material Compound 21a instead of compound 1d. The title product was used in the next step without further purification (99.3%).

¹H-NMR (CDCl₃, δ): 0.93-1.41 (m, 6H); 1.47-2.01 (m, 6H); 2.62-2.91 (m, 1H); 3.14-3.29 (m, 1H); 3.41-3.60 (m, 1H); 3.71-4.03 (m, 2H); 5.01-5.32 (m, 2H); 5.73-5.98 (m, 1H); 7.08-7.41 (m, 5H); 9.56-9.69 (m,1H).

1-(4-Allyloxy-4-cyclohexyl-3-phenylbutyl)-4-[5-(2,3-dihydro-1,4-benzodioxinyl)]piperazine

The title compound was prepared using the method described for the Compound of Example 1 but using compound 21b instead of Compound 1e and 1-(2,3-dihydro-1,4-benzodioxin-5-yl)piperazine instead of 1-(2,2,2-trifluoroethoxyphenyl)piperazine. The crude was purified by flash chromatography (PE:Me₂CO 7:3) to afford the title compound (64.1%).

¹H-NMR (CDCl₃, δ): 0.98-1.40 (m, 6H); 1.48-1.98 (m, 6H); 1.88-2.00 (m, 2H); 2.08-2.31 (m, 3H); 2.39-2.71 (m, 4H); 2.2.78-2.94 (m, 1H); 2.96-3.21 (m, 5H); 3.72-4.06 (m, 2H); 4.68-4.87 (m, 4H); 5.05-5.34 (m, 2H); 5.81-6.02 (m, 1H); 6.47-6.63 (m, 2H); 6.80-6.88 (m, 1H); 7.11-7.37 (m, 5H).

[M+H]⁺=491

### EXAMPLE 22

### 1-(4-Allyloxy-4-cyclohexyl-3-phenylbutyl)-4-(4-fluoro-2-methoxyphenyl)piperazine

The title compound was prepared using the method described for the Compound of Example 1 but using compound 21b instead of Compound 1e and 1-(4-fluoro-2-methoxyphenyl)piperazine instead of 1-(2,2,2-trifluoroethoxyphenyl)piperazine. The crude was purified by flash chromatography (PE:Me₂CO 7:3) to afford the title compound (77.1%).

¹H-NMR (CDCl₃, δ): 0.98-1.41 (m, 6H); 1.47-2.01 (m, 6H); 2.09-2.28 (m, 3H); 2.41-2.70 (m, 4H); 2.79-2.92 (m, 1H); 2.93-3.09 (m, 4H); 3.11-3.22 (m, 1H); 3.77-3.89 (m, 4); 3.39-4.08 (m, 1H); 5.07-5.34 (m, 2H); 5.79-6.01(m, 1H); 6.51-6.68 (m, 2H); 6.69-6.92 (m, 1H); 7.13-7.37 (m, 5H).

[M+H]⁺=481

### EXAMPLE 23

### 1-(4-Cyclohexyl-3-phenyl-4-propargyloxybutyl)-4-[5-(2,3-dihydro-1,4-benzodioxinyl)]piperazine

4-Cyclohexyl-3-phenyl-4-propargyloxybutyraldehyde dimethyl acetal (Compound 23a)

The title compound was synthesised as described for Compound 1d using as a starting material Compound 17c instead of Compound 1c and propargyl bromide instead of methyl iodide. The reaction mixture was stirred for 8 h at r.t. and 5 h at 45° C. Et₂O extraction and purification by column chromatography (PE-EtOAc 85:15) yielded the title compound (50%).

¹H-NMR (CDCl₃, δ): 0.90-1.41 (m, 6H); 1.48-1.74 (m, 5H); 1.75-1.89 (m, 1H); 1.90-2.04 (m, 1H); 2.18-2.37 (m, 1H); 2.38-2.44 (m, 1H); 2.88-3.04 (m, 1H); 3.21 (d, 6H); 3.90-4.17 (m, 3H); 7.12-7.37 (m, 5H).

4-Cyclohexyl-3-phenyl-4-propargyloxy -3-phenylbutyraldehyde (Compound 23b)

The title compound was obtained following the procedure described for Compound 1e, but using as a starting material Compound 23a instead of Compound 1d. The title product was used in the next step without further purification (99%).

¹H-NMR (CDCl₃, δ): 0.81-1.41 (m, 6H); 1.49-1.90 (m, 5H); 2.39-2.51 (m, 1H); 2.66-2.88 (m, 1H); 2.89-3.08 (m, 1H); 3.31-3.42 (m, 1H); 3.43-6.59 (m, 1H); 3.97-4.19 (m, 2H); 7.12-7.39 (m, 5H); 9.57-9.69 (m, 1H).

1-(4-Cyclohexyl-3-phenyl-4-propargyloxybutyl)-4-[5-(2,3-dihydro-1,4-benzodioxinyl)]piperazine

The title compound was prepared using the method described for the Compound of Example 1 but using compound 23b instead of Compound 1e and 1-(2,3-dihydro-1,4-benzodioxin-5-yl)piperazine instead of 1-(2,2,2-trifluoroethoxyphenyl)piperazine. The crude was purified by flash chromatography (PE:Me₂CO 7:3) to afford the title compound (67.1%).

¹H-NMR (CDCl₃, δ): 0.98-1.39 (m, 6H); 1.48-1.99 (m, 6H); 2.11-2.29 (m, 3H); 2.39-2.46 (m, 1H); 2.47-2.71 (m, 4H); 2.82-3.96 (m, 1H); 2.97-3.12 (m 4H); 3.17-3.29 (m, 1H); 3.95-4.16 (m, 2H); 4.17-4.38 (m, 4H); 6.48-6.72 (m, 2H); 6.69-6.83 (m, 1H); 7.12-7.35 (m, 5H)

[M+H]⁺=489

### EXAMPLE 24

### 1-(4-Cyclohexyl-3-phenyl-4-propargyloxybutyl)-4-(4-fluoro-2-methoxyphenyl)piperazine

The title compound was prepared using the method described for the Compound of Example 1 but using Compound 23b instead of Compound 1e and 1-(4-fluoro-2-methoxyphenyl)piperazine instead of 1-(2,2,2-trifluoroethoxyphenyl)piperazine. The crude was purified by flash chromatography (PE:Me₂CO 8:2) to afford the title compound (66.2%).

¹H-NMR (CDCl₃, δ): 0.99-1.41 (m, 6H); 1.51-2.00 (m, 6H); 2.11-2.29 (m, 3H); 2.39-2.46 (m, 1H); 2.47-2.70 (m, 4H); 2.78-3.12 (m, 5H); 3.13-3.29 (m, 1H); 3.81 (s, 3H); 3.96-4.17 (m, 2H); 6.51-6.67 (m, 2H); 6.79-7.94 (m, 1H); 7.11-7.34 (m, 5H).

[M+H]⁺=479

### EXAMPLE 25

### 1-(4-Cyclohexyl-3-phenyl-4-propoxybutyl)-4-[5-(2,3-dihydro-1,4-benzodioxinyl)]piperazine

4-Cyclohexyl-3-phenyl-4-propoxybutyraldehyde dimethyl acetal (Compound 25a) The title compound was synthesised as described for Compound 1d using as a starting material Compound 17c instead of Compound 1c and propyl bromide instead of methyl iodide. The reaction mixture was stirred for 8 h at r.t. and 5 h at 45° C. Et₂O extraction and purification by column chromatography (PE-EtOAc 85:15) yielded the title compound (32.7%).

¹H-NMR (CDCl₃, δ): 0.91 (t, 3H); 0.99-1.32 (m, 6H); 1.45-1.98 (m, 10H); 2.19-2.38 (m, 1H); 2.83-2.99 (m, 1H); 3.01-3.10 (m, 1H); 3.16-3.29 (m, 5H); 3.31-3.5 (m, 1H); 3.91-4.08 (m, 1H); 7.13-7.34 (m, 5H).

4-Cyclohexyl-3-phenyl-4-propoxybutyraldehyde (Compound 25b)

The title compound was obtained following the procedure described for Compound 1e, but using as a starting material Compound 25a instead of compound 1d. The title product was used in the next step without further purification (99.3%).

¹H-NMR (CDCl₃, δ): 0.80-0.95 (m, 3H); 0.96-1.37 (m, 6H); 1.41-1.88 (m, 7H); 2.57-3.09 (m, 3H); 3.11-3.59 (m, 3H); 7.11-7.39 (m, 5H); 9.10-9.15 (m, 1H).

1-(4-Cyclohexyl-3-phenyl-4-propoxybutyl)-4-[5-(2,3-dihydro-1,4-benzodioxinyl)]piperazine

The title compound was prepared using the method described for the Compound of Example 1 but using Compound 25b instead of Compound 1e and 1-(2,3-dihydro-1,4-benzodioxin-5-yl)piperazine instead of 1-(2,2,2-trifluoroethoxyphenyl)piperazine. The crude was purified by flash chromatography (PE:Me₂CO 8:2) to afford the title compound (50.7%).

¹H-NMR (CDCl₃, δ): 0.93 (t, 3H); 0.99-1.37 (m, 6H); 1.44-1.98 (m, 8H); 2.10-2.31 (m, 3H); 2.41-2.69 (m, 4H); 2.72-2.90 (m, 1H); 2.95-3.18 (m, 5H); 3.20-3.98 (m, 2H); 4.19-4.37 (m, 4H); 6.48-6.67 (m, 2H); 6.69-6.83 (m, 1H); 7.11-7.36 (m, 5H).

[M+H]⁺=493

### EXAMPLE 26

### 1-[5-(2,3-Dihydro-1,4-benzodioxinyl)]-4-(4-hydroxy-3-phenyl)hexylpiperazine

4-Oxo-2-phenylbutyronitrile(Compound 26a)

The title compound was obtained following the procedure described for Compound 1e but using as a starting material Compound 17a instead of compound 1d. After the usual work-up procedure, the so obtained title compound was used without further purification in the next step.

¹H-NMR (CDCl₃, δ): 2.94-3.29 (m, 2H), 4.31-4.45 (m, 1H), 7.30-7.48 (m, 5H), 9.78 (bs, 1H).

1-(3-Cyano-3-phenylpropyl)-4-(2,3-dihydrobenzo-1,4-dioxin-5-yl)piperazine (Compound 26b)

The title compound was obtained following the procedure described for the compound of Example 1 but using as a starting material Compound 26a instead of compound 1e and 1-(2,3-dihydro-1,4-benzodioxin-5-yl)piperazine instead of 1-(2,2,2-trifluoroethoxyphenyl)piperazine. Purification by flash chromatography (PE-EtOAc 4:6) yielded the title compound (85%). Oil.

¹H-NMR (CDCl₃, δ): 2.01-2.29 (m, 2H), 2.31-2.72 (m, 6H), 3.02-3.22 (m, 4H), 4.03-4.18 (m, 1H), 4.19-4.38 (m, 4H), 6.50-6.62 (m, 2H), 6.73-6.85 (m, 1H), 7.31-7.42 (m, 5H).

1-(2,3-Dihydrobenzo-1,4-dioxin-5-yl)-4-(3-formyl-3-phenylpropyl)piperazine (Compound 26c)

The title compound was obtained following the procedure described for Compound 6f (alternative method) but using as a starting material Compound 26b instead of Compound 6h. Purification by flash chromatography (CH₂Cl₂-MeOH 95:5) yielded the title compound (60%). Oil.

¹H-NMR (CDCl₃, δ): 1.88-2.02 (m, 1H), 2.30-2.51 (m, 3H), 2.52-2.98 (m, 4H), 2.99-3.31 (m, 4H), 3.63-3.77 (m, 1H), 4.20-4.41 (m, 4H), 6.48-6.67 (m, 2H), 6.68-6.85 (m, 1H), 7.21-7.43 (m, 5H), 9.79 (bs, 1H).

1-[5-(2,3-Dihydro-1,4-benzodioxinyl)]-4-(4-hydroxy-3-phenylhexyl)piperazine

The title compound was obtained following the procedure described for the compound of Example 6 but using as a starting material Compound 26c instead of Compound 6f. The crude was purified by flash chromatography (CH₂Cl₂-MeOH/NH₃ 97:3) affording the title product as a yellow glassy oil (22.6%).

¹H-NMR (CDCl₃, δ): 0.79-1.01 (m, 3H) 1.02-1.79 (m, 4H), 1.80-1.98 (m, 1H), 1.99-2.24 (m, 1H), 2.26-2.96 (m 6H), 2.98-3.33 (m, 4H), 3.41-3.79 (m, 1H), 4.18-4.38 (m, 4H), 6.45-6.68 (m, 2H), 6.69-6.87 (m, 1H), 7.19-7.38 (m, 5H).

[M+H]⁺=397.4

### EXAMPLE 27

### 1-[5-(2,3-Dihydro-1,4-benzodioxinyl)]-4-(4-hydroxy-3-phenyl)heptyl]piperazine

The title compound was obtained together with the compound of Example 12 as the main impurity. It was purified by flash chromatography (EtOAc-MeOH/NH₃ 95:5).

¹H-NMR (CDCl₃, δ): 0.78-0.92 (m, 3H), 1.15-1.3 (m, 5H), 1.80-2.08 (m, 2H), 2.28-2.40 (m, 2H), 2.52-2.83 (m, 5H), 3.02-3.18 (m, 4H), 3.65-3.79 (m, 1H), 4.16-4.32 (m, 4H), 6.48-6.62 (m, 2H), 6.71-6.82 (t, 1H), 7.15-7.33 (m, 5H).

### EXAMPLE 28

### 1-[5-(2,3-Dihydro-1,4-benzodioxinyl)]-4-(4-hydroxy-3-phenylhex-5-enyl]piperazine

The title compound was obtained following the procedure described for the compound of Example 6 but using as a starting material Compound 26c instead of Compound 6f and vinylmagnesium chloride (1M THF sol.) instead of ethylmagnesium chloride. The crude was purified by flash chromatography (CH₂Cl₂-MeOH/NH₃ 97:3) affording the title product as a yellow glassy oil (66%).

¹H-NMR (CDCl₃, δ): 1.35-1.85 (br, 1H), 1.86-2.28 (m, 2H), 2.30-2.91 (m, 7H), 2.98-3.25 (m, 4H), 3.43-3.81 (m, 1H), 4.19-4.40 (m, 4H), 4.90-5.35 (m, 2H), 5.66-5.89 (m, 1H), 6.46-6.69 (m, 2H), 6.71-6.85 (m, 1H), 7.14-7.42 (m, 5H).

[M+H]⁺=395.3

### EXAMPLE 29

### 1-[5-(2,3-Dihydro-1,4-benzodioxinyl)]-4-(4-hydroxy-5-methyl-3-phenyl)hexyl]piperazine

The title compound was obtained following the procedure described for the compound of Example 6 but using as a starting material Compound 26c instead of Compound 6f and isopropylmagnesium chloride (2M THF sol.) instead of ethylmagnesium chloride. The crude was purified by flash chromatography (CH₂Cl₂-MeOH/NH₃ 98:2) affording the title product as a white solid (35%).

¹H-NMR (CDCl3, δ): 0.73-0.95 (m, 6H), 1.30-1.48 (m, 1H), 1.76-1.95 (m, 1H), 1.96-2.21 (m, 1H), 2.22-2.48 (m, 2H), 2.49-2.95 (m, 5H), 2.96-3.28 (m, 4H), 3.52-3.73 (m, 1H), 4.19-4.41 (m, 4H), 5.02-5.68 (bs, 1H), 6.49-6.63 (m, 2H), 6.75-6.87 (m, 1H), 7.15-7.39 (m, 5H).

[M+H]⁺=411.7

### EXAMPLE 30

### 1-[5-(2,3-Dihydro-1,4-benzodioxinyl)]-4-(4-hydroxy-3-phenyl)pentyl]piperazine

The title compound was obtained following the procedure described for the compound of Example 6 but using as a starting material Compound 26c instead of Compound 6f and methylmagnesium bromide (3M THF sol.) instead of ethylmagnesium chloride. The crude was purified by flash chromatography (CH₂Cl₂-MeOH/NH₃ 99:1) affording the title product as a white solid (42%) characterized as a 7:3 (RS,RS)-(RS,SR) mixture.

¹H-NMR (CDCl₃, δ): 0.85-1.15 (m, 3H), 1.41-1.67 (m, 2H), 1.74-1.95 (m, 1H), 1.96-2.24 (m, 1H), 2.25-3.29 (m, 10H), 3.81-3.99 (m, 1H), 4.19-4.39 (m, 4H), 6.50-6.65 (m, 2H), 6.72-6.87 (m, 1H), 7.14-7.41 (m, 5H).

[M+H]⁺=383.6

### EXAMPLE 31

### 1-[5-(2,3-Dihydro-1,4-benzodioxinyl)]-4-(4-hydroxy-3-phenylhept-5-ynyl)piperazine

The title compound was obtained following the procedure described for the compound of Example 6 but using as a starting material Compound 26c instead of Compound 6f and 1-propynylmagnesium bromide (0.5 M THF sol.) instead of ethylmagnesium chloride. The crude was purified by flash chromatography (CH₂Cl₂-MeOH/NH₃ 99:1) affording the title product as a pale yellow solid (35%).

¹H-NMR (CDCl₃, δ): 1.72-1.89 (m, 3H), 1.91-2.21 (m, 2H), 2.30-2.50 (m, 2H), 2.51-2.82 (m, 4H), 2.83-3.24 (m, 5H), 3.51-3.73 (m, 1H), 4.20-4.41 (m, 4H), 4.42-4.61 (m, 1H), 6.48-6.63 (m, 2H), 6.73-6.82 (m, 1H), 7.20-7.39 (m, 5H).

[M+H]⁺=407.4

### EXAMPLE 32

### 1-[5-(2,3-Dihydro-1,4-benzodioxinyl)]-4-(4-hydroxy-3-phenylhept-5-enyl)piperazine

The title compound was obtained following the procedure described for the compound of Example 6 but using as a starting material Compound 26c instead of Compound 6f and 1-propenylmagnesium bromide (0.5 M THF sol.) instead of ethylmagnesium chloride. The crude was purified by flash chromatography (CH₂Cl₂-MeOH/NH₃ 99:1) affording the title product as a pale yellow solid (83%).

¹H-NMR (CDCl3, _{δ}): 1.31-2.31 (m, 8H), 2.32-2.91 (m, 6H), 2.92-3.28 (m, 4H), 4.17-4.33 (m, 4H), 5.23-5.75 (m, 2H), 6.48-6.63 (m, 2H), 6.71-6.84 (m, 1H), 7.12-7.39 (m, 5H).

[M+H]⁺=409.6

### EXAMPLE 33

### 1-[5-(2,3-Dihydro-1,4-benzodioxinyl)]-4-(4-hydroxy-3-phenylhex-5-ynyl)piperazine

The title compound was obtained following the procedure described for the compound of Example 6 but using as a starting material Compound 26c instead of Compound 6f and 1-ethynyl magnesium bromide (0.5 M THF sol.) instead of ethylmagnesium chloride. The crude was purified twice, first by flash chromatography (CH₂Cl₂-MeOH/NH₃ 99:1) followed by preparative LC affording the title product as a white solid (8%).

¹H-NMR (CDCl₃, δ): 1.11-1.99 (br, 1H), 2.01-2.25 (m, 2H), 2.27-2.31 (m, 1H), 2.34-2.37 (m, 2H), 2.61-2.82 (m, 4H), 2.85-3.22 (m, 5H), 4.18-4.32 (m, 4H), 6.47-6.62 (m, 2H), 6.74-6.86 (m, 1H), 7.19-7.41 (m, 5H).

[M+H]⁺=393.7

### EXAMPLE 34

### 1-[5-(2,3-Dihydro-1,4-benzodioxinyl)]-4-(4-hydroxy-3-phenylhept-6-enyl)piperazine

The title compound was obtained following the procedure described for the compound of Example 6 but using as a starting material Compound 26c instead of Compound 6f and allylmagnesium bromide (1 M THF sol.) instead of ethylmagnesium chloride. The crude was purified by flash chromatography (CH₂Cl₂-MeOH/NH₃ 99:1) affording the title product as a brownish oil (27%).

¹H-NMR (CDCl₃, δ): 1.41-1.72 (br, 1H), 1.73-2.25 (m, 4H), 2.26-2.50 (m, 2H), 2.51-2.91 (m, 5H), 3.03-3.24 (m, 4H), 3.78-3.92 (m, 1H), 4.20-4.39 (m, 4H), 4.92-5.17 (m, 2H), 5.73-5.95 (m, 1H), 6.51-6.64 (m, 2H), 6.67-6.84 (m, 1H), 7.11-7.40 (m, 5H).

[M+H]⁺=409.7

### EXAMPLE 35

### 1-[5-(2,3-Dihydro-1,4-benzodioxinyl)]-4-(4-hydroxy-6-methyl-3-phenylhept-5-enyl)piperazine

The title compound was obtained following the procedure described for the compound of Example 6 but using as a starting material Compound 26c instead of Compound 6f and 2-methyl-1-propenylmagnesium bromide (0.5 M THF sol.) instead of ethylmagnesium chloride. The crude was doubly purified by flash chromatography (CH₂Cl₂-MeOH/NH₃99:1) followed by preparative LC affording the title product as a white solid (10%).

¹H-NMR (CDCl₃, δ): 1.12-1.85 (m, 8H), 1.87-2.02 (m, 1H), 2.03-2.29 (m, 1H), 2.30-2.91 (m, 6H), 2.93-3.21 (m, 4H), 4.17-4.35 (m, 4H), 4.36-4.48 (m, 1H), 4.96-5.22 (m, 1H), 6.48-6.62 (m, 2H), 6.75-6.85 (m, 1H), 7.12-7.38 (m, 5H).

[M+H]⁺=423.8

### EXAMPLE 36

### 1-[5-(2,3-Dihydro-1,4-benzodioxinyl)]-4-(4-hydroxy-6-methyl-3-phenyl)heptyl]piperazine

To a solution of 0.708 ml of 2 M isobutyl magnesium chloride (in THF) in 3 ml of THF was added 0.226 g of LiClO₄. The mixture was stirred at r.t. for 1 h; afterwards, 0.13 g of Compound 26c in 3 ml of THF was added dropwise. The reaction mixture was allowed to stir at r.t. for 3 h then quenched with an aq. saturated solution of NH₄Cl, alkalinised and extracted with EtOAc. The combined extracts were dried (Na₂SO₄) and evaporated to dryness. The crude was purified by flash chromatography (CH₂Cl₂-MeOH 99:1) affording the title product as an ivory white solid (54.6%).

¹H-NMR (CDCl₃, δ): 0.68-1.01 (m, 8H), 1.12-1.38 (m, 2H), 1.65-1.85 (m, 1H), 2.03-2.29 (m, 1H), 2.30-2.52 (m, 1H), 2.53-2.83 (m, 2H), 2.84-3.47 (m, 9H), 3.73-3.92 (m, 1H), 4.18-4.39 (m, 4H), 6.49-6.65 (m, 2H), 6.67-6.87 (m, 1H), 7.10-7.40 (m, 5H).

[M+H]⁺=425.2

### EXAMPLE 37

### 1-[5-(2,3-dihydro-1,4-benzodioxinyl)]-4-(4-hydroxy-3-phenylbutyl]piperazine

The title compound was obtained together with the compound of Example 36. Purification by flash chromatography afforded the title product as an oil.

¹H-NMR (CDCl₃, δ): 1.36-1.64 (m, 1H), 1.81-2.05 (m, 2H), 2.50-2.73 (m, 4H), 2.75-2.93 (m, 2H), 2.94-3.27 (m, 4H), 3.62-3.72 (m, 2H), 4.20-4.39 (m, 4H), 6.10-6.45 (br, 1H), 6.46-6.62 (m, 2H), 6.70-6.83 (m, 1H), 7.13-7.37 (m, 5H).

[M+H]⁺=369.7

### EXAMPLE 38

### (RS,SR)-1-[5-(2,3-Dihydro-1,4-benzodioxinyl)]-4-(4-hydroxy-3-phenylpentyl)piperazine

The title compound was obtained together with the compound of Example 11. Purification by flash chromatography afforded the title product as an oil, characterized as the pure (RS,SR) diastereomer.

¹H-NMR (CDCl₃, δ): 0.85-1.12 (m, 3H), 1.80-2.21 (m, 3H), 2.32-2.45 (m, 2H), 2.46-2.83 (m, 5H), 2.95-3.17 (m, 4H), 3.92-4.01 (m, 1H), 4.18-4.37 (m, 4H), 6.45-6.61 (m, 2H), 6.72-6.85 (m, 1H), 7.11-7.38 (m, 5H).

[M+H]⁺=383.6

### EXAMPLE 39

### 1-[4-Cyclohexyl-3-(2-dimethylaminocarbonylphenyl)-4-oxobutyl]-4-[5-(2,3-dihydro-1,4-benzodioxinyl)]piperazine

2-(2-Cyclohexyl-2-oxoethyl)-N,N-dimethylbenzamide (Compound 39a)

The title compound was obtained as described for Compound 13a. after the usual work-up procedure, the crude was purified by flash chromatography (PE-EtOAc 1:1) to afford the title compound (34.6%).

¹H-NMR (CDCl₃, δ): 1.11-1.50 (m, 5H); 1.61-1.98 (m, 6H); 2.84 (d,3H); 3.04 (d, 3H); 3.81-4.02 (m, 2H); 7.11-7.42 (m, 5H).

[M+H]⁺=274

4-Cyclohexyl-3-(2-dimethylaminocarbonyl)-4-oxobutyraldehyde dimethyl acetale (Compound 39b)

The title compound was prepared using the method described for Compound 2b but using Compound 39a instead of Compound 2a. The crude was purified by flash chromatography (PE-Me2CO 75:25). Yield: 21.3%.

¹H-NMR (CDCl₃, δ): 0.94-1.49 (m, 5H); 1.51-1.83 (m, 5H); 1.84-2.01 (m, 1H); 1.36-1.69 (m, 2H); 2.88 (s, 3H); 3.18 (s, 3H); 3.31 (d, 6H); 4.12-4.34 (m, 2H); 7.12-7.39 (m, 4H).

4-Cyclohexyl-3-(2-dimethylaminocarbonyl)-4-oxobutyraldehyde (Compound 39c)

The title compound was obtained following the procedure described for Compound 1d, but using as a starting material Compound 39b instead of Compound 1c. The crude was used in the next step without further purification.

¹H-NMR (CDCl₃, δ): 0.90-2.12 (m, 12 H); 2.60-3.32 (m, 8H); 4.39-4.58 (m, 1H); 7.04-7.51 (m, 4H); 9.63-9.72 (m, 1H)

1-[4-Cyclohexyl-3-(2-dimethylaminocarbonylphenyl)-4-oxobutyl]-4-[5-(2,3-dihydro-1,4-benzodioxinyl)]piperazine

The title compound was prepared using the method described for the Compound of Example 1 but using Compound 39c instead of Compound 1e and 1-(2,3-dihydro-1,4-benzodioxin-5-yl)piperazine instead of 1-(2,2,2-trifluoroethoxyphenyl)piperazine. The crude was purified by flash chromatography (PE:Me₂CO 6:4) to afford the title compound (65%).

¹H-NMR (CDCl₃, δ): 1.03-2.01 (m, 11H); 2.23-2.42 (m, 3H); 2.44-2.72 (m, 5H); 2.91 (s, 3H); 3.97-3.13 (m, 4H); 3.19 (s, 3H); 4.13-4.38 (m, 5H); 6.48-6.63 (m, 2H); 6.70-6.84 (m, 1H); 7.13-7.39 (m, 4H).

[M+H]⁺=520

### EXAMPLE 40

### 1-[4-Cyclohexyl-3-(2-dimethylaminocarbonylphenyl)-4-hydroxybutyl]-4-[5-(2,3-dihydro-1,4-benzodioxinyl)]piperazine

The title compound was synthesised using the method described for Compound 1c but starting from the Compound of Example 39 instead of Compound 1b. After the usual work-up procedure, the crude was purified by flash chromatography (PE-Me₂CO—NH₃/MeOH 7:3:0.2) affording the title compound (65.2%).

[M+H]⁺=522.45

### EXAMPLE 41

### 1-[4-Cyclohexyl-3-(2-dimethylaminocarbonylphenyl)-4-oxobutyl]-4-(4-fluoro-2-methoxyphenyl)piperazine

The title compound was prepared using the method described for the Compound of Example 1 but using Compound 39c instead of Compound 1e and 1-(4-fluoro-2-methoxyphenyl)piperazine instead of 1-(2,2,2-trifluoroethoxyphenyl)piperazine. The crude was purified by flash chromatography (PE:Me₂CO 6:4) to afford the title compound (64.5%).

¹H-NMR (CDCl₃, δ): 1.03-2.02 (m, 11H); 2.17-2.37 (m, 3H); 2.41-2.73 (m, 5H); 2.94 (s, 3H); 2.95-3.12 (m, 4H); 3.17 (s, 3H); 3.85 (s, 3H); 4.11-4.27 (m, 1H); 6.51-6.69 (m, 2H); 6.78-6.92 (m, 1H); 7.12-7.41 (m, 4H).

[M+H]⁺=510

### EXAMPLE 42

### 1-[4-Cyclohexyl-3-(2-dimethylaminocarbonylphenyl)-4-hydroxybutyl]-4-(4-fluoro-2-methoxyphenyl)piperazine

The title compound was synthesised using the method described for Compound 1c but starting from the Compound of Example 41 instead of Compound 1b. After the usual work-up procedure, the crude was purified by flash chromatography (PE-Me₂CO—NH₃/MeOH 75:25:0.2) affording the title compound (64.2%).

[M+H]⁺=512.6

### EXAMPLE 43

### 1-[3-(2-Cyanophenyl)-4-oxopentyl]-4-[5-(2,3-dihydro-1,4-benzodioxinyl)]piperazine

### 3-(2-Cyanophenyl)-4-oxopentanaldehyde diethyl acetal (Compound 43a)

The title compound was prepared using the method described for Compound 2b but using 1-(2-cyanophenyl)propan-2-one (R. A. Bruce, Org. Prep. Proc. Int. 407-412, 1999) instead of Compound 2a. The crude was purified by flash chromatography (PE-EtOAc 8:2). Yield: 13%.

¹H-NMR (CDCl₃, δ): 1.10-1.29 (m, 6H), 1.87-2.04 (m, 1H), 2.14 (s, 3H), 2.42-2.59 (m, 1H), 3.31-3.71 (m, 4H), 4.28-4.43 (m, 2H), 7.30-7.41 (m, 2H), 7.51-7.72 (m, 2H).

3-(2-Cyanophenyl)-4-oxopentanaldehyde (Compound 43b)

The title compound was obtained following the procedure described for Compound 1d, but using as a starting material Compound 43b instead of Compound 1c. The crude was used in the next step without further purification.

1-[3-(2-Cyanophenyl)-4-oxopenttyl]-4-[5-(2,3-dihydro-1,4-benzodioxinyl)]piperazine

The title compound was prepared using the method described for the Compound of Example 1 but using Compound 43b instead of Compound 1e and 1-(2,3-dihydro-1,4-benzodioxin-5-yl)piperazine instead of 1-(2,2,2-trifluoroethoxyphenyl)piperazine. The crude was purified by flash chromatography (PE:EtOAc 6:4) to afford the title compound (43.8%).

¹H-NMR (CDCl₃, δ): 1.75-1.92 (m, 1H), 2.21 (s, 3H), 2.30-2.41 (m, 3H), 2.41-2.69 (m, 4H), 2.92-3.12 (m, 4H), 4.19-4.40 (m, 5H), 6.48-6.62 (m, 2H), 6.71-6.82 (t, 1H), 7.30-7.43 (m, 2H), 7.51-7.72 (m, 2H).

### EXAMPLE 44

### 1-[4-Cyclohexyl-3-(2-trifluoromethoxyphenyl)-4-oxobutyl]-4-(4-indolyl)piperazine

1-Cyclohexyl-2-(2-trifluoromethoxyphenyl)ethanone (Compound 44a)

A mixture of 1.26 g of 2-trifluoromethoxybenzyl chloride, Zn powder (0.59 g) and 1,2-DME was refluxed for 3 h, cooled at r.t.; afterwards, it was filtered and was added 0.002 g of dichlorobis(triphenylphosphine)palladium (II) followed by 0.72 ml of cyclohexanecarbonyl chloride to the filtrate stirred at r.t. Afterwards, the reaction mixture was stirred at reflux for 4 h, cooled at r.t. After the usual work-up procedure (see Compound 1a), the crude was purified by flash chromatography (methyl tert-butyl ether-PE 96:4) to afford 0.22 g of the title compound.

¹H-NMR (CDCl₃, δ): 1.10-2.00 (m, 10H); 3.80 (s, 3H); 7.18-7.40 (m, 4H);

4-Cyclohexyl-4-oxo-3-(2-trifluoromethoxyphenyl)butyraldehyde diethyl acetal (Compound 44b)

To a solution of 0.22 g of Compound 44a in 1 ml of DMSO was added potassium tert-butoxide (0.091 g) at r.t. After 15 min 0.12 ml of 2-bromoacetaldehyde diethyl acetal was added and the reaction mixture was heated at 50° C. for 5 h. Afterwards, it was cooled to r.t., diluted with water and extracted with methyl tert-butyl ether, which was dried (Na₂SO₄) and evaporated to dryness in vacuo giving a crude which was purified by flash chromatography (methyl tert-butyl ether-PE 93:7) affording 0.092 g of the pure title product.

¹H-NMR (CDCl₃, δ): 1.00-2.10 (m,17H); 2.10-2.45 (m, 2H); 3.75 (q,4H); 4.32 (t,1H); 4,50 (t,1H); 7.10-7.40 (m, 4H)

4-Cyclohexyl-4-oxo-3-(2-trifluoromethoxyphenyl)butfraldehyde (Compound 44c)

0.09 g of Compound 44b, 1.1 ml of 1N HCl and 5 ml of acetone were stirred at r.t. for 4 h. Evaporation and extraction with CH₂Cl₂ afforded the title compound, which was used in the next step without further purification.

¹H-NMR (CDCl₃, δ): 0.80-1.95 (m,9H); 1.95-2.15 (m,1H); 2.25-2.45 (m,1H); 2.52 (dd,1H); 3.40 (dd,1H); 4.80 (dd,1H); 7.10-7.40 (m,4H); 9.75 (s,1H)

1-[4-Cyclohexyl-3-(2-trifluoromethoxyphenyl)-4-oxobutyl]-4-(4-indolyl)piperazine

The title compound was prepared using the method described for the Compound of Example 1 but using Compound 44c instead of Compound 1e and 1-(4-indolyl)piperazine instead of 1-(2,2,2-trifluoroethoxyphenyl)piperazine. The crude was purified by flash chromatography (PE:EtOAc 6:4) to afford the title compound (53%).

¹H-NMR (CDCl₃, δ): 1.00-2.10 (m,11H); 2.20-2.50 (m,4H); 2.50-2.80 (m,4H); 3.15-3.40 (m,4H); 4.50 (t,1H); 6.50 (d,1H); 7.60 (dd,1H); 7.007.20 (m,3H); 7.20-7.35 (m,4H); 8.15 (s,1H)

### EXAMPLE 45

### (RS,SR) 1-[4-Acetoxy-4-cyclohexyl-3-(2-fluorophenyl)butyl]-4-(2-methoxyphenyl)piperazine

1-Cyclohexyl-2-(2-fluorophenyl)ethanone (Compound 45a)

To a mixture of 36 ml of 2-fluorobenzylzinc chloride (0.5 M sol. in THF) and 0.008 g of dichlorobis(triphenylphosphine)palladium (II) stirred at 0° C. was added dropwise via a syringe 2.14 ml of cyclohexanecarbonyl chloride. Afterwards, the reaction mixture was stirred at r.t. for 4 h, quenched with an aqueous saturated solution of ammonium chloride (25 ml), extracted with 20 ml of EtOAc, which was dried (Na₂SO₄) and evaporated to dryness in vacuo affording 3.52 g of the title compound as a crude, which could be used in the following step without further purification.

¹H-NMR(CDCl₃, δ): 1.10-2.05 (m, 10H), 2.47 (tt,1H), 3.77 (s, 2H), 6.97-7.32 (m, 4H)

4-Cyclohexyl-4-oxo-3-(2-fluorophenyl)butyraldehyde diethyl acetal (Compound 45b)

A solution of 5.02 g of compound 45a in 136 ml of toluene was heated at reflux recovering 35 ml of toluene by distillation to remove water. Afterwards, 3.18 g of potassium tert-butoxide was added and stirring at reflux was continued for 30 min.; the reaction mixture was cooled to 80° C. and 4.27 ml of 2-bromoacetaldehyde diethyl acetal was added. After 18 h at reflux, the reaction mixture was cooled to r.t., quenched with an aqueous saturated solution of ammonium chloride (30 ml), extracted with 30 ml of EtOAc, which was dried (Na₂SO₄) and evaporated to dryness in vacuo giving a crude which was purified by flash chromatography (petroleum ether-EtOAc 92.5:7.5) affording 2.97 g of the pure title product.

¹H-NMR (CDCl₃, δ): 1.00-2.10 (m, 17H), 2.20-2.52 (m, 2H), 3.30-3.72 (m, 4H) 4.25-4.45 (m, 2H), 6.90-7.35 (m, 4H)

4-Cyclohexvl-4-oxo-3-(2-fluorophenyl)butyraldehyde (Compound 45c)

A mixture of 1.12 g of the compound 45b, 9 ml of 50% aq. trifluoroacetic acid and 18 ml of CH₂Cl₂ was stirred for 2 h at r.t., and then diluted with 10 ml of CH₂Cl₂. The organic layer was separated, washed with brine (2×15 ml), dried (Na₂SO₄) and evaporated to dryness in vacuo to afford a crude (0.88 g), used in the next step without further purification.

¹H-NMR (CDCl₃, δ): 0.90-2.10 (m, 10H), 2.25-2.70 (m, 2H), 3.12-3.52 (m, 1H), 4.60-4.80 (m, 1H), 6.95-7.40 (m, 4H), 9.75 (s, 1H)

1-[4-Cyclohexyl-3-(2-fluorophenyl)-4-oxobutyl]-4-(2-methoxyphenyl)piperazine (Compound 45d)

A mixture of 0.88 g of the compound 45c, 0.84 g of 1-(2-methoxyphenyl)piperazine HCl, 1.06 g of sodium triacetoxyborohydride and 33 ml of CH₂Cl₂ was stirred at r.t. for 1 h, kept overnight resting, alkalinised with 20% aq. Na₂CO₃. The organic layer was separated, washed with brine (2×30 ml), dried (Na₂SO₄) and evaporated to dryness in vacuo the give a crude (1.46 g) which was used in the next step without further purification. A sample was purified by flash chromatography (petroleum ether-EtOAc 6:4) affording a pure sample.

¹H-NMR (CDCl₃, δ): 1.05-2.00 (m, 11H), 2.20-2.44 (m, 4H), 2.45-2.72 (m, 4), 2.90-3.20 (m, 4H), 3.85 (s, 3H)

(RS,SR)-1-[4-Cyclohexyl-3-(2-fluorophenyl)-4-hydroxybutyl]-4-(2-methoxyphenyl)piperazine (Compound 45e)

To a solution of 1.46 g of Compound 45d in 33 ml of MeOH stirred at 0° C. was added 0.19 g of NaBH₄ and the mixture was stirred at r.t. for 4 h. The solvent was evaporated and the reaction crude was taken up with H₂O and extracted with EtOAc. The organic layer was separated, washed with brine (2×15 ml), dried (Na₂SO₄) and evaporated to dryness in vacuo to give a crude which was purified by sequential flash chromatography (petroleum ether-EtOAc-2 N ammonia in methanol 75:25:2; petroleum ether-EtOAc-2 N ammonia in methanol 80:20:2) affording 0.82 g of Compound 45e (upper TLC Rf; eluent:petroleum ether-EtOAc-2 N ammonia in methanol 70:30:2).

¹H-NMR (CDCl₃, δ): 0.80-1.40 (m, 6H), 1.50-1.82 (m, 4H),1.85-2.10(m, 3 H), 2.21-2.45 (m, 2H), 2.52-2.85 (m, 4H), 2.98-3.26 (m, 4H), 3.28-3.42 (m, 1H), 3.50-3.60 (m, 1H), 3.85 (s, 3H), 6.80-7.30 (m, 7H), 7.62-7.80 (m, 1H); OH peak not detectable

(1R,2S) 1-Cyclohexyl-4-[4-(2-methoxyphenyl)piperazin-1-yl]-2-(2-fluorophenyl)butan-1-ol (Compound 45eA)

This compound was obtained by chiral column chromatography on Compound 45e using Chiralpak AD (0.46×25 cm), eluting with n-hexane-EtOH 95:5 (flow=0.5 ml/min; detector UV 247 nm).

(1S,2R) 1-Cyclohexyl-4-[4-(2-methoxyphenyl)piperazin-1-yl]-2-(2-fluorophenyl)butan-1-ol (Compound 45eB)

This compound was obtained by chiral column chromatography on Compound 45e using Chiralpak AD (0.46×25 cm), eluting with n-hexane-EtOH 95:5 (flow=0.5 ml/min; detector UV 247 nm).

The absolute stereochemistry of Compounds 45eB, in form of its salts with hydrogen bromide, was determined by single crystal x-ray diffraction, as follows.

Single crystal X-ray diffraction experiment:

A needle shape single crystal was selected for X-ray diffraction analysis and mounted on a glass fiber. The data were collected on Rigaku Rapid cylinder shape image plate X-ray area detector with detector aperture=45.0×25.6 cm. It was controlled by a Windows 2000 based PC computer with Rapid Auto version 1.06 software (Rigaku, 2000), at low temperature (−120° K), with Micromax-002 micro-Confocal mirrors CutKα radiation [λ(CuKα)=1.5405 Å]. Indexing was performed from three 3° oscillations frames that were exposed for 360 seconds. All reflections were measured in five image groups with six frames in each group; the exposure time was 160 seconds per degree. Among them, five groups of images were at angles phi=0°, 90°, 180°, 270° with chi=50° and phi=0° with chi=0° all frames were delta omega=30°, and which makes the 2θmax=136.30°. The sample/detector distance was 12.74 cm. The data reduction program, Rapid Auto version 1.06 (Rigaku, 2000), determined the Laue group was −1, and total 7,986 reflections were integrated for structure solution and refinements.

Single Crystal Results:

The structure was solved by direct methods, using SIR92 (Altomare et al. 1994). All calculations were performed using the CrystalStructure 3.0 (MSC/Rigaku, 2002; Watkin et al., 1996, Carruthers and Watkin, 1979) crystallographic software package. The trial solution obtained 38 nohydrogen atoms in the asymmetrical unit. Least squares refinement included all nonhydrogen atomic coordinates and anisotropic thermal parameters. The final cycle of full-matrix least-squares refinement on F was based on 6,297 reflections with I>3σ(I), converged with agreement factors: R=0.071, S=2.224, Rw=0.073. The absolute configuration was determined by using the calculated Flack×parameter, which was 0.00 with esd=0.04. Expected values are 0.0 (within 3 esd's) for correct and +1.0 for inverted absolute structure.

### References

Altomare, A., Cascarano, G., Giacovazzo, C. Gualgliardi, A., Burla, M., Polidori, G., and Camalli, M., (1994) SIR92, J. Appl. Cryst., 27, 435.

Carruthers, J. R. and Watkin, D. J. (1979), Acta Cryst, A35, 698-699.

Rigaku (2000), Rapid Auto, Rigaku Corporation, Tokyo, Japan.

Rigaku and Rigaku/MSC, (2000-2002), Crystal Structure Analysis Software, CrystalStructure Version 3.00, Rigaku/MSC, 9009 New Trails Drive, The Woodlands, Tex., USA 77381-5209. Rigaku, 3-9-12 Akishima, Tokyo 196-8666, Japan.

Watkin, D. J., Prout, C. K. Carruthers, J. R. & Betteridge, P. W., CRYSTALS Issue 10, Chemical Crystallography Laboratory, Oxford, UK.

(RS,SR) 1-[4-Acetoxy-4-cyclohexyl-3-(2-fluorophenyl)butyl]-4-(2-methoxyphenyl)piperazine

To a solution of 0.135 g of Compound 45e and 0.043 ml of TEA in 5 ml of CH₂Cl₂ stirred at 0-5° C., was added 0.021 ml of acetyl chloride. Afterrwards, the reaction mixture was stirred at r.t. for 4 h, washed with 5% aq. NaHCO₃ 5%(1×10 ml), H2O (2×15 ml), dried on Na₂SO₄ and evaporated in vacuo to afford the title product (0,143 g)

¹H-NMR (CDCl₃, δ): δ:0.80-1.25 (m,5H); 1.25-1.50 (m,1H); 1.50-1.90 (m, 7H); 1.95 (s, 3H); 2.10-2.40 (m,2H); 2.40-2.65 (m, 4H); 2.90-3.15 (m, 4H); 3.38-3.55 (m,1H); 3.85 (m, 3H); 5.05 (t,1H); 6.80-7.25 (m,7H); 7.30-7.45 (m,1H)

### EXAMPLE 46

### (RS,SR) 1-[4-Cyclohexyl-3-(2-fluorophenyl)-4-methoxycarbonyloxybutyl]-4-(2-methoxyphenyl)piperazine

To a solution of 0.112 g of Compound 45e in 0.8 ml of pyridine stirred at 0° C., was added 0.022 ml of methyl chloroformate. The reaction mixture was stirred at r.t. for 4 days and at 40° C. for 5 h. Additional 0.045 ml of methyl chloroformate was added, heating at 40° C. for 4 h. After 3 days at r.t., methyl chloroformate (0.045 ml) was added and the mixture stirred at r.t. for 6 h. After cooling, it was poured into water and extracted with EtOAc(2×15 ml), washed with 2×5 ml of H₂O, dried (Na₂SO₄) and evaporated to dryness in vacuo. The crude was purified by flash chromatography to (PE-EtOAc-NH₃/MeOH 75:25:2.5) to yield 0.022 g of the title product.

¹H-NMR (CDCl₃, δ): 0.80-2.05 (m,13H); 2.10-2.40 (m,2H); 2.40-2.75 (m,4H); 2.90-3.20 (m,4H); 3.38-3.60 (m,1H); 3.70 (m,3H); 3.85 (m,3H); 4.88 (t,1H); 6.80-7.25 (m,7H); 7.38-7.50 (m,1H)

### EXAMPLE 47

### (RS,SR) 1-[4-Cyclohexyl-4-ethylaminocarbonyloxy-3-(2-fluorophenyl)butyl]-4-(2-methoxyphenyl)piperazine

To a solution of 0.126 g of Compound 45e in 0.5 ml of pyridine stirred at 0° C., was added 0.113 of ethyl isocyanate. The reaction mixture was stirred at r.t. for 24 h and at 50° C. for 3 h. After cooling, it was poured into water and extracted with Et₂O,washed with H₂O, dried (Na₂SO₄) and evaporated to dryness in vacuo to afford 0.108 g of the title product.

¹H-NMR (CDCl₃, δ): 0.90-1.40(m, 9H); 1.60-2.10 (m,7H); 2.10-2.40 (m, 2H); 2.50-2.65 (m, 4H); 2.95-3.30 (m, 6H); 3.40-3.55 (m,1H); 3.85 (s, 3H); 4.50 (t,1H); 4.90 (t,1H); 6.80-7.22 (m,7H); 7.32-7.45 (m,1H)

### EXAMPLE 48

### (RS,SR) 1-[4-Aminocarbonyloxy-4-cyclohexyl-3-(2-fluorophenyl)butyl]-4-(2-methoxyphenyl)piperazine

To a solution of 0.124 g of Compound 45e in 5 ml of CH₂Cl₂ was added potassium cyanate. To the suspension stirred at r.t. was added 0.087 ml of trifluoroacetic acid. After 24 h at r.t. and 5 h at 40° C., additional trifluoroacetic acid was added (0,17 ml). After 6 h at 40° C., the reaction misture was cooled, evaporated to dryness, diluted with water and NaOH 2 N, extracted with EtOAC; the extracvt was washed with H₂O, dried (Na₂SO₄) and evaporated to dryness in vacuo. The crude was purified by flash chromatography to (PE-EtOAc-NH₃/MeOH 75:25:2.5) to yield 0.064 g of the title product.

¹H-NMR (CDCl₃, δ): 0.90-1.50 (m,6H); 1.50-2.05 (m,7H); 2.10-2.40 (m,2H); 2.50-2.70 (m,4H); 2.95-3.15 (m,4H); 3.40-3.55 (m,1H); 3.85 (s,3H); 4.45 (s,2H); 4.85-4.95 (m,1H); 6.80-7.26 (m,7H);7.32-7.45 (m,1H)

### EXAMPLE 49

### 1-[5-(2,3-Dihydro-1,4-benzodioxinyl)]-4-(4-hydroxy-5,5-dimethyl-3-phenyl)hexyl]piperazine

The title compound was synthesised following the procedure described for the compound of Example 36 but using tert-butylmagnesium chloride (1N in THF) instead of isobutylmagnesium chloride. The mixture was stirred at r.t. for 3 h. The crude was purified by flash chromatography (CH₂Cl₂-MeOH 99:1) affording the title product as an brownish solid (15%).

¹H-NMR (CDCl₃, δ): 0.88 (m, 9H), 1.31-1.48 (m, 2H), 1.73-1.97 (m, 1H), 1.96-2.2 (m, 1H), 2.22-2.48 (m, 2H), 2.49-2.97 (m, 5H), 2.99-3.28 (m, 4H), 3.61-3.73 (m, 4H), 6.49-6.63 (m, 2H), 6.75-6.87 (m, 1H), 7.15-7.44 (m, 5H).

[M+H]⁺=425.7

### EXAMPLE 50

### 1-(4-Fluoro-2-methoxyphenyl)-4-[(4-hydroxy-3-phenyl)hept-5-ynyl]piperazine

1-(3-Cyano-3-phenylpropyl)-4-(4-fluoro-2-methoxyphenyl)piperazine (Compound 50a) The title compound was obtained following the procedure described for the compound of Example 1 but using as a starting material Compound 26a instead of Compound 1e and 1-(4-fluoro-2-methoxyphenyl)piperazine instead of 1-(2,2,2-trifluoroethoxyphenyl)piperazine. Purification by flash chromatography (PE-EtOAc 4:6) yielded the title compound (90%). Oil.

¹H-NMR (CDCl₃, δ): 1.94-2.29 (m, 2H); 2.36-2.79 (m, 6H); 2.97-3.14 (m, 4H); 3.85 (s, 3H); 4.01-4.12 (m, 1H); 6.54-6.71 (m, 2H); 6.82-7.97 (m, 1H); 7.28-7.47 (m, 5H)

1-(4-Fluoro-2-methoxyphenyl)-4-(3-formyl-3-phenylpropyl)piperazine (Compound 50b)

The title compound was obtained following the procedure described for Compound 6f (alternative method) but using as a starting material Compound 50a instead of Compound 6h. Purification by flash chromatography (CH₂Cl₂-MeOH 95:5) yielded the title compound (55%). Oil.

¹H-NMR (CDCl₃, δ): 1.81-2.00 (m, 1H); 2.24-2.48 (m, 3H); 2.49-3.82 (m, 4H); 3.87-3.19 (m, 4H); 3.61-3.73 (m, 1H); 3.84 (s; 3H); 6.53-6.71 (m, 2H); 6.80-6.94 (m, 1H); 7.12-7.45 (m, 5H); 9.68-9.79 (m, 1H).

1-(4-Fluoro-2-methoxyphenyl)-4-[(4-hydroxy-3-phenyl)hept-5-ynyl]piperazine

The title compound was obtained following the procedure described for the compound of Example 36 but using as a starting material Compound 50b instead of Compound 26c and using 1-propynylmagnesium bromide (0.5 N in THF) instead of a solution of isobutylmagnesium chloride. The crude was purified by flash chromatography (CH₂Cl₂-MeOH 99:1) affording the title product as a yellow glassy oil (22.6%).

¹H-NMR (CDCl₃, δ): 0.84-1.17 (m, 3H), 1.40-1.70 (m, 2H), 1.74-1.90 (m, 1H), 1.93-2.22 (m, 1H), 2.25-3.29 (m, 10H), 3.80 (s, 3H), 3.84-3.99 (m, 1H), 6.50-6.65 (m, 2H), 6.70-6.90 (m, 1H), 7.14-7.38 (m, 5H).

[M+H]⁺=397.2

### EXAMPLE 51

### (E,Z)-1-(4-Fluoro-2-methoxyphenyl)-4-[(4-hydroxy-3-phenyl)hept-5-enyl]piperazine (1:1 mixture)

The title compound was obtained following the procedure described for the compound of Example 36 but using as a starting material Compound 50b instead of Compound 26c and using 1-propenylmagnesium bromide (0.5 N in THF) instead of a solution of isobutylmagnesium chloride.

The crude was purified by flash chromatography (CH₂Cl₂-MeOH 99:1) affording the title compound as a yellow glassy oil (30%).

¹H-NMR (CDCl₃, δ): 1.17-1.73 (m, 4H), 1.92-2.51 (m, 2H), 2.52-3.42 (m, 11H), 3.83 (s, 3H), 4.22-4.65 (m, 1H), 5.28-5.75 (m, 2H), 6.50-6.64 (m, 2H), 6.72-6.91 (m, 1H), 7.13-7.41 (m, 5H).

[M+H]⁺=399.2

Further purification by preparative LC-MS chromatography afforded the isolation of the following compounds:

### EXAMPLE 52

### (E,Z)-1-(4-fluoro-2-methoxyphenyl)-4-[(4-hydroxy-3-phenyl)hept-5-enyl]piperazine (5:95 mixture)

¹H-NMR (CDCl₃, δ): 1.24-1.43 (m, 3H), 1.74-1.91 (m, 2H), 1.92-3.12 (m, 12H), 3.82 (s, 3H), 4.41-4.55 (m, 1H), 5.18-5.65 (m, 2H), 6.48-6.59 (m, 2H), 6.71-6.91 (m, 1h), 7.13-7.43 (m, 5H).

[M+H]⁺=399.2

### EXAMPLE 53

### (E)-1-(4-Fluoro-2-methoxyphenyl)-4-[(4-hydroxy-3-phenyl)hept-5-enyl]piperazine (RS,RS:RS,SR 9:1 mixture)

¹H-NMR (CDCl₃, δ): 1.44-1.59 (m, 4H), 1.71-2.03 (m, 2H), 2.32-3.15 (m, 11H), 3.84 (s, 3H), 4.09-4.12 (m, 1H), 5.15-5.23 (m, 1H), 5.34-5.49 (m, 1H), 6.48-6.60 (m, 2H), 6.70-6.92 (m, 1H), 7.13-7.43 (m, 5H).

[M+H]⁺=399.2

### EXAMPLE 54

### 1-[5-(2,3-Dihydro-1,4-benzodioxinyl)]-4-[(4-hydroxy-5-methyl-3-phenyl)hex-5-enyl]piperazine (RS,RS:RS,SR 6:4)

The title compound was synthesised following the procedure described for the compound of Example 36 but using isopropenylmagnesium bromide (0.5 N in THF) instead a solution of isobutylmagnesium chloride. The mixture was stirred at r.t. for 3 h. The crude was purified by flash chromatography (CH₂Cl₂-MeOH 99:1) affording the title product as an brownish solid (38%).

¹H-NMR (CDCl₃, δ): 1.65-1.79 (m, 3H), 2.03-2.12 (m, 1H), 2.32-2.48 (m, 1H), 2.57-2.78 (m, 1H), 2.79-3.42 (m, 12H), 4.21-4.45 (m, 4H), 4.65-4.95 (m, 2H), 6.49-6.58 (m, 1H), 6.60-6.65 (m, 1H), 6.72-6.79 (m, 1H), 7.19-7.41 (m, 5H).

[M+H]⁺=409.6

### EXAMPLE 55

### 1-[5-(2,3-Dihydro-1,4-benzodioxinyl)]-4-[(4-hydroxy-6-methyl-3-phenyl)hept-6-enyl]piperazine

The title compound was synthesised following the procedure described for the compound of Example 36 but using 2-methylallylmagnesium chloride (0.5 N in THF) instead a solution of isobutylmagnesium chloride. The mixture was stirred at r.t. for 3 h. The crude was purified by flash chromatography (CH₂Cl₂-MeOH 99:1) affording the title product as an brownish solid (48%).

¹H-NMR (CDCl₃, δ): 1.68-1.73 (m, 3H), 1.94-2.05 (m, 2H), 2.06-2.18 (m, 1H), 2.19-2.31 (m, 1H), 2.47-2.51 (m, 1H), 2.52-3.42 (m, 12H), 4.23-4.44 (m, 4H), 4.62-4.93 (m, 2H), 6.49-6.53 (m, 1H), 6.62-6.65 (m, 1H), 6.77-6.84 (m, 1H), 7.21-7.48 (m, 5H).

[M+H]⁺=423.6

### EXAMPLE 56

### 1-[5-(2,3-Dihydro-1,4-benzodioxinyl)]-4-[4-hydroxy-4-(2-thienyl)-3-phenylbutyl]piperazine

The title compound was synthesised following the procedure described for the compound of Example 36 but using 2-thienylmagnesium bromide (1 M in THF) instead of a solution of isobutylmagnesium chloride. The mixture was stirred at r.t. for 3 h. The crude was purified by flash chromatography (CH₂Cl₂-MeOH 99:1) affording the title product as an brownish solid (33%).

¹H-NMR (CDCl₃, δ): 1.75-2.23 (m, 3H), 2.48-2.53 (m, 2H), 2.54-2.94 (m, 4H), 3.05-3.32 (m, 5H), 4.23-4.41 (m, 4H), 5.12-5.21 (m, 1H), 6.50-6.93 (m, 5H), 7.11-7.48 (m, 6H).

[M+H]⁺=451.7

### EXAMPLE 57

### 1-[5-(2,3-Dihydro-1,4-benzodioxinyl)]-4-[(4-hydroxy-3-phenyl)octyl]piperazine

The title compound was synthesised following the procedure described for the compound of Example 36 but using n-butylmagnesium chloride (2 M in THF) instead of a solution of isobutylmagnesium chloride. The mixture was stirred at r.t. for 3 h. The crude was purified by flash chromatography (CH₂Cl₂-MeOH 99:1) affording the title product as an brownish solid (48%).

¹H-NMR (CDCl₃, δ): 0.61-0.78 (m, 3H), 1.02-1.31 (m, 6H), 1.94-2.06 (m, 1H) 2.26-2.48 (m, 1H), 2.51-2.61 (m, 2H), 2.62-3.32 (m, 10H), 3.62-3.74 (m, 1H), 4.23-4.44 (m, 4H), 6.47-6.50 (m, 1H), 6.61-6.65 (m, 1H), 6.79-6.84 (m, 1H), 7.21-7.51 (m, 5H).

[M+H]⁺=425.4

### EXAMPLE 58

### 1-(4-Fluoro-2-methoxyphenyl)-4-[(4-methoxy-3-phenyl)hept-5-ynyl]piperazine

4-Hydroxy-3-phenylhept-5-ynaldehyde dimethyl acetal (Compound 58a)

The title compound was obtained following the procedure described for the compound of Example 6 but using as a starting material Compound 17b instead of compound 6f and 1-propynylmagnesium chloride (2M sol. in THF) instead of ethylmagnesium chloride. The crude was purified by flash chromatography (EtOAc-PE 3:7) affording the title product. (81%).

¹H-NMR (CDCl₃, δ): 1.78-1.88 (m, 3H), 1.91-2.21 (m, 3H), 2.92-3.08 (m, 1H), 3.20-3.35 (m, 7H), 4.18-4.24 (m, 1H), 7.20-7.39 (m, 5H).

4-Methoxy-3-phenylhept-5-ynaldehyde dimethyl acetal (Compound 58b)

The title compound was synthesized as described for Compound 1d using as a starting material Compound 58a instead of Compound 1c. After EtOAc extraction, the crude was used without further purification in the next step.

4-Methoxy-3-phenylhept-5-ynaldehyde (Compound 58c)

The title compound was obtained following the procedure described for Compound 1e, but using as a starting material Compound 58b instead of compound 1d. The title product was used in the next step without further purification.

1-(4-Fluoro-2-methoxyphenyl)-4-[(4-methoxy-3-phenyl)hept-5-ynyl]piperazine

The title compound was prepared using the method described for the Compound of Example 1 but using compound 58c instead of Compound 1e and 1-(4-fluoro-2-methoxyphenyl)piperazine instead of 1-(2,2,2-trifluoroethoxyphenyl)piperazine. The crude was purified by flash chromatography (PE-EtOAc 3:7) to afford the title compound (17.5%).

¹H-NMR (CDCl₃, δ):.1.68 and1.83 (2 s, 3H); 1.88-2.05 (m, 2H), 2.15-2.48 (m, 4H), 2.48-2.78 (m, 4H), 2.81-2.90 (m, 1H); 2.90-3.15 (m, 2H); 3.25 and 3.40 (2×s, 3H); 3.76 (m, 3H); 3.93-3.98 (m, 1H), 6.48-6.57 (m, 2H), 6.76 (dd, 1H) 7.22-7.48 (m, 5H).

[M+H]⁺=411.13

### EXAMPLE 59

### (E,Z)-1-(4-Fluoro-2-methoxyphenyl)-4-[(4-methoxy-3-phenyl)hept-5-enyl]piperazine (upper TLC rf diastereomer)

4-Hydroxy-3-phenylhept-5-enaldehyde dimethyl acetal (Compound 59a)

The title compound was obtained following the procedure described for the compound of Example 6 but using as a starting material Compound 17b instead of compound 6f and 1-propenylmagnesium chloride (2M sol. in THF) instead of ethylmagnesium chloride. The crude was purified by flash chromatography (PE-EtOAc 7:3) affording the title product. (42%).

¹H-NMR (CDCl₃, δ): 1.50 and 1.60 (2 d, 3H), 1.85-2.04 (m, 2H), 2.15-2.38 (m, 1H), 2.78-2.92 (m, 1H), 3.22 and 3.38 (4×s, 6H), 4.10-4.24 (m, 1H), 4.58 (dd, 1H), 5.22-5.59 (m, 2H), 7.20-7.39 (m, 5H).

4-Methoxy-3-phenylhept-5-enaldehyde dimethyl acetal (Compound 59b)

The title compound was synthesised as described for Compound 1d using as a starting material Compound 59a instead of Compound 1c. After EtOAc extraction, the crude was used in the next step without further purification.

¹H-NMR (CDCl₃, δ): 1.10 and 1.75 (m, 3H), 1.85-2.35 (m, 2H), 2.63-3.05 (m, 1H), 3.12-3.52 (m, 9H), 4.10-4.24 (m, 1H), 4.58 (dd, 1H), 5.22-5.59 (m, 2H), 7.12-7.39 (m, 5H).

4-Methoxy-3-phenylhept-5-enaldehyde (Compound 59c)

The title compound was obtained following the procedure described for Compound 1e, but using as a starting material Compound 59b instead of compound 1d. The title product was used in the next step without further purification.

1-(4-Fluoro-2-methoxyphenyl)-4-[(4-methoxy-3-phenyl)hept-5-enyl]piperazine

The title compound was prepared using the method described for the Compound of Example 1 but using compound 59c instead of Compound 1e and 1-(4-fluoro-2-methoxyphenyl)piperazine instead of 1-(2,2,2-trifluoroethoxyphenyl)piperazine. The crude was purified by flash chromatography (PE:EtOAc 4:6) to afford the title compound (13.2%).

¹H-NMR (CDCl₃, δ): 1.45 and1.63 (2 d, 3H); 1.68-1.81 (m, 1H), 1.88-2.05 (m, 1H), 2.08-2.27 (m, 2H), 2.42-2.58 (m, 4H); 2.63-2.75 (m, 1H); 2.88-3.00 (m, 4H), 3.08 and 3.13 (2×s, 3H); 3.52 and 4.05 (2 d, 1H); 3.75 (s, 3H), 5.05-5.23 (m, 1H), 5.47-5.71 (m, 1H), 6.48-6.57 (m, 2H), 6.76 (m, 1H), 7.05-7.32 (m, 5H).

[M+H]⁺=413.34

### EXAMPLE 60

### (E,Z)-1-(4-Fluoro-2-methoxyphenyl)-4-[(4-methoxy-3-phenyl)hept-5-enyl]piperazine (lower TLC rf diastereomer)

The title compound was isolated during the purification step of Example 59.

¹H-NMR (CDCl₃, δ): 1.48 and1.60 (2d, 3H); 1.79-2.12 (m, 2H), 2.18-2.40 (m, 2H), 2.22-2.78 (m, 5H); 2.92-3.13 (m, 4H); 3.25 and 3.30 (2s, 3H); 3.57 and 4.08 (2t, 1H); 3.85 (s, 3H), 5.11-5.23 (m, 1H), 5.39-5.65 (m, 1H), 6.58-6.69 (m, 2H), 6.82-6.91 (m, 1H), 7.15-7.35 (m, 5H).

[M+H]⁺=413.34

### EXAMPLE 61

### 1-[4-Cyclohexyl-3-(2-methoxymethylphenyl)-4-oxobutyl]-4-(4-fluoro-2-methoxyphenyl)piperazine

2-Methoxymethylbenzyl bromide (Compound 61a)

A mixture of 1.2 g of 2-methoxymethylbenzyl alcohol (J.Chem. Soc., 1954, 2819-2826), 2.5 g of triphenylphosphine, 3.14 g of tetrabromomethane and 50 ml of CH₂Cl₂ was stirred at r.t. for 2 h. Afterwards, the reaction mixture was evaporated to dryness in vacuo and purified by flash chromatography (CH₂Cl₂) to afford 1.62 g of the title compound.

¹H-NMR (CDCl₃, δ): 3.42 (d, 3H), 4.60 (s, 2H), 4.65 (s, 2H), 7.15-7.30 (m, 3H), 7.22-7.45 (m, 4H)

1-Cyclohexyl-2-(2-methoxymethylphenyl)ethanone (Compound 61b)

To a suspension of 1.44 g of Zn(Cu) (prepared as described in Org. Syn. 5,855) in 5 ml of anhydrous benzene stirred at r.t. under nitrogen, was added dropwise a solution of 1.6 g of Compound 61a and 1.17 ml of N,N-dimethylacetamide in 10 ml of benzene. The mixture was stirred at r.t. for 1 h, then at reflux for 4 h. After cooling at 60° C., was added a solution of 0.073 g of palladium tetrakis triphenylphosphine in 3 ml of benzene followed by a solution of 0.55 ml of cyclohexanecarbonyl chloride in 3 ml of benzene. The reaction mixture was stirred 2.5 h at r.t. After overnight resting, the mixture was diluted with EtOAc and filtered on a celite panel; the filtrate was washed with a aq. saturated solution of ammonium chloride, aq. NaHCO₃ and brine, dried and evaporated to dryness. The crude was purified by flash chromatography (PE-EtOAc 100:4) to afford 1 g of the title compound.

¹H-NMR (CDCl₃, δ): 1.10-2.05 (m, 10H), 2.25-2.48 (m, 1H), 3.40 (s, 3H), 4.50 (s, 2H), 5.18 (s, 2H), 7.25-7.50 (m, 4H)

4-Cyclohexyl-4-oxo-3-(2-methoxymethylphenyl)butyraldehyde diethyl acetal (Compound 61c)

The title compound was prepared using the method described for Compound 2b but using Compound 61b instead of 1-(2-trifluoromethoxyphenyl)propan-2-one. Usual work-up procedure and purification afforded the title compound.

4-Cyclohexyl-4-oxo-3-(2-methoxymethylphenyl)butyraldehyde (Compound 61d)

The title compound was obtained following the procedure described for Compound 1e, but using as a starting material Compound 61c instead of compound 1d. The title product was used in the next step without further purification.

1-[4-Cyclohexyl-3-(2-methoxymethylphenyl)-4-oxobutyl]-4-(4-fluoro-2-methoxyphenyl)piperazine

The title compound was prepared using the method described for the Compound of Example 1 but using Compound 61d instead of Compound 1e and 1-(4-fluoro-2-methoxyphenyl)piperazine instead of 1-(2,2,2-trifluoroethoxyphenyl)piperazine. The crude was purified by flash chromatography (PE:EtOAc 6:4) to afford the title compound.

[M+H]⁺=483.6

### EXAMPLE 62

### 1-[4-Cyclohexyl-4-hydroxy-3-(2-methoxymethylphenyl)-butyl]-4-(4-fluoro-2-methoxyphenyl)piperazine

The title compound was synthesised using the method described for Compound 1c but starting from the compound of Example 61 instead of Compound1b. The title compound was isolated after the usual work-up procedure.

[M+H]⁺=485.5

### EXAMPLE 63

### 1-[5-(2,3-Dihydro-1,4-benzodioxinyl)]-4-[4-cyclohexyl-3-(2-methoxymethylphenyl)-4-oxobutyl]-piperazine

The title compound was prepared using the method described for the Compound of Example 1 but using Compound 61d instead of Compound 1e and 1-[5-(2,3-dihydro-1,4-benzodioxinyl)]piperazine instead of 1-(2,2,2-trifluoroethoxyphenyl)piperazine. The crude was purified by flash chromatography to afford the title compound.

[M+H]⁺=493.7

### EXAMPLE 64

### 1-[4-Cyclohexyl-4-hydroxy-3-(2-methoxymethylphenyl)-butyl]-4-(2,3-dihydro-1,4-benzodioxinyl)piperazine

The title compound was synthesised using the method described for Compound 1c but starting from the compound of Example 63 instead of Compound 1b. The title compound was isolated after the usual work-up procedure.

[M+H]⁺=495.5

### EXAMPLE 65

### (RS,SR) 1-[4-Cyclohexyl-4-methylaminothiocarbonyloxy-3-(2-fluorophenyl)butyl]-4-(2-methoxyphenyl)piperazine

To a solution of 0.088 g of Compound 45e in 0.6 ml of pyridine stirred at 0° C., was added 0.073 g of methyl isothiocyanate. The reaction mixture was stirred at r.t. for 24 h and at 100° C. for 10 h. After cooling, it was poured into water and extracted with Et₂O,washed with H₂O, dried (Na₂SO₄) and evaporated to dryness in vacuo. Purification by flash chromatography afforded 0.04 g of the title product.

[M+H]⁺=514.4

### EXAMPLE 66

### Radioligand Binding to Recombinant 5-HT_{1A} Receptors A. Method

Genomic clone coding for the human 5-HT_{1A}-serotonergic receptor was stably transfected in a human cell line (HeLa). HeLa cells were grown as monolayers in Dulbecco's modified Eagle medium (DMEM), containing 10% foetal bovine serum, gentamycin (0.1 mg/ml) and 5% carbon dioxide, at 37° C. The cells were detached from the growth flask at 95% confluence by a cell scraper and were lysed in cold 5 mM Tris and 5 mM EDTA buffer (pH 7.4). The homogenates were centrifuged at 40000×g×20 minutes and the pellets were resuspended in a small volume of cold 5 mM Tris and 5 mM EDTA buffer (pH 7.4) and immediately frozen and stored at −70° C. until use. On the day of experiment, the cell membranes were resuspended in incubation buffer: 50 mM Tris HCl (pH 7.4), 2.5 mM MgCl₂, 10 mM pargyline (Fargin et al., Nature 335, 358-360, 1988). The membranes were incubated in a final volume of 1 ml for 30 minutes at 30° C. with 1 nM [³H]8-OH-DPAT, in the absence or presence of the test compounds. Non-specific binding was determined in the presence of 10 μM 5-HT. Incubation was stopped by addition of cold Tris-HC1 buffer and rapid filtration through a 0.2%-polyethyleneimine-pretreated Whatman-GF/B or Schleicher-&-Schuell-GF52 filter.

B. Results

The affinities of the tested compounds were evaluated as inhibition of specific binding of the radioligand to 5-HT_{1A} receptors (IC₅₀) by using the non-linear curve-fitting program Allfit (De Lean et al., Am. J. Physiol. 235, E97-E102 (1978). The IC₅₀ value was converted to an affinity constant (Ki) by the equation of Cheng & Prusoff (Cheng Y. C., Prusoff W. H., Biochem. Pharmacol. 22, 3099-3108 (1973)).

The results reported in Table 1 show that the compounds of the invention tested had a high affinity for the 5-HT_{1A} receptor.

TABLE 1
Binding affinity for 5-HT_{1A} receptors

| Example | Ki (nM) |
|---|---|
| 1 | 1.45 |
| 5 | 8.31 |
| 6 | 3.66 |
| 7 | 7.27 |
| 9 | 1.90 |
| 10 | 1.68 |
| 11 | 3.65 |
| 12 | 3.01 |
| 13 | 0.53 |
| 14 | 0.401 |
| 14a | 0.878 |
| 14b | 0.965 |
| 15 | 1.69 |
| 16 | 0.90 |
| 17 | 0.64 |
| 18 | 0.81 |
| 19 | 0.69 |
| 20 | 3.19 |
| 21 | 0.77 |
| 22 | 2.03 |
| 23 | 0.63 |
| 24 | 0.64 |
| 25 | 1.18 |
| 26 | 2.62 |
| 27 | 1.09 |
| 28 | 2.92 |
| 29 | 1.08 |
| 30 | 5.43 |
| 31 | 2.71 |
| 32 | 3.57 |
| 33 | 5.03 |
| 34 | 1.67 |
| 35 | 1.47 |
| 36 | 0.56 |
| 37 | 5.03 |
| 39 | 2.92 |
| 40 | 1.84 |
| 42 | 4.87 |
| 44 | 1.14 |
| 45 | 0.54 |
| 46 | 0.72 |
| 47 | 0.85 |
| 48 | 0.36 |

### EXAMPLE 67

### Effects on Rhythmic Bladder-voiding Contractions Induced by Bladder Filling in Anaesthetised Rats

A. Method

Female Sprague-Dawley rats weighing 225-275 g (Crl: CD® (SD) IGS BR, Charles River Italia) were used. The animals were housed with free access to food and water and maintained on a forced 12-hour alternating light-dark cycle at 22-24° C. for at least one week, except during the experiment. The activity on rhythmic bladder voiding contractions was evaluated according to the method of Dray (Dray J., Pharmacol. Methods, 13:157, 1985), with some modifications as in Guarneri (Guarneri, Pharmacol. Res. 27:173, 1993). Briefly, rats were anaesthetised by subcutaneous injection of 1.25 g/kg (5 ml/kg) urethane, after which the urinary bladder was catheterised via the urethra using PE 50 polyethylene tubing filled with physiological saline. The catheter was tied in place with a ligature around the external urethral orifice and was connected to conventional pressure transducers (Statham P23 ID/P23 XL). The intravesical pressure was displayed continuously on a chart recorder (Battaglia Rangoni KV 135 with DCI/TI amplifier). The bladder was then filled via the recording catheter by incremental volumes of warm (37° C.) saline until reflex bladder-voiding contractions occurred (usually 0.8-1.5 ml). For intravenous injection of bioactive compounds, PE 50 polyethylene tubing filled with physiological saline was inserted into the jugular vein.

From the cystometrogram, the number of contractions recorded 15 minutes before (basal values) and after treatment, as well as the mean amplitude of these contractions (mean height of the peaks in mmHg), was evaluated.

Since most compounds produced an effect that was relatively rapid in onset and lead to a complete cessation of bladder contractions, bioactivity was conveniently estimated by measuring the duration of bladder quiescence (i.e., the length of the time during which no contractions occured). The number of tested animals showing a reduction in the number of contractions higher than 30% of that observed in the basal period was also recorded.

To compare the potency of the tested compounds for inhibiting the bladder voiding contractions, equieffective doses that result in the disappearance of contractions for a time of 10 minutes (ED₁₀ₘᵢₙ) were computed by means of linear regression using the least square method. The extrapolated doses which induces a reduction in the number of contractions greater than 30% in 50% of the treated rats (ED₅₀) was evaluated by the method of Bliss (Bliss C. I., Quart J. Pharm. Pharmacol. 11, 192-216, 1938).

B. Results

The rapid distension of the urinary bladder in urethane-anaesthetised rats produced a series of rhythmic bladder-voiding contractions whose characteristics have been described (Maggi et al., Brain Res. 380:83, 1986; Maggi et al., J. Pharmacol. Exp. Ther., 230: 500, 1984). The frequency of these contractions is related to the sensory afferent arm of reflex micturition and to the integrity of the micturition centre, while their amplitude depends on the function of the reflex efferent arm. In this model system, compounds that act mainly on the central nervous system (such as morphine) cause a block in voiding contractions, whereas drugs that act at the level of the detrusor muscle, such as oxybutynin, lower the amplitude of the bladder contractions.

The results obtained are shown in table 2.

TABLE 2
Effects on rhythmic bladder-voiding contractions after
intravenous administration

| | | ED₅₀ | ED₅₀ |
|---|---|---|---|
| | ED_{10 min} | (frequency) | (amplitude) |
| Example | μg/kg | μg/kg | μg/kg |
| Ex. 45 | 584 | 127 | n.a. |
| Morphine | 50 | 30 | n.a. |
| Oxybutynin | 7770 | >10000 | 240 |

n.a. = not active; no significant reduction of the height of the peaks

Data represent the ED₁₀ₘᵢₙ values (the extrapolated dose inducing 10 minutes of disappearance of the contractions), the ED₅₀ (frequency) values (the extrapolated doses inducing a reduction of the number of contractions >30% in 50% of treated rats), and the ED₅₀ (amplitude) values (the extrapolated doses inducing a 30% reduction of amplitude of the contractions in 50% of treated rats).

The compounds of the present invention inhibited the frequency of micturition, with no effects on their amplitude.

### EXAMPLE 68

### Effect on Cystometric Parameters in Conscious Rats After Oral Administration

A. Method

Male Sprague-Dawley rats[Crl: CD® (SD) IGS BR] of 300-400 g supplied by Charles River Italia are used. The animals are housed with free access to food and water and maintained on a forced 12-hour-light/12-hour-dark cycle at 22-24° C. of temperature, except during the experiment. To quantify urodynamic parameters in conscious rats, cystometrographic studies are performed according to the procedure previously reported (Guarneri et al., Pharmacol. Res. 24: 175, 1991).

Briefly, rats are anaesthetised by intraperitoneal administration of 3 ml/kg of Equithensin solution (pentobarbital 30 mg/kg and chloral hydrate 125 mg/kg) and placed in a supine position. An approximately-10-mm-long midline incision are made in the shaved and cleaned abdominal wall. The urinary bladder is gently freed from adhering tissues, emptied and then cannulated viaan incision in the bladder body, using a polyethylene cannula (0.58-mm internal diameter, 0.96-mm external diameter) which had been permanently sutured with silk thread. The cannula is exteriorised through a subcutaneous tunnel in the retroscapular area, where it is connected to a plastic adapter in order to avoid the risk of removal by the animal. For drug testing, the rats are utilised one day after implantation.

On the day of the experiment, the rats are placed in modified Bollman cages, i.e., restraining cages that are large enough to permit the rats to adopt a normal crouched posture, but narrow enough to prevent turning around. After a stabilisation period of about 20 minutes, the free tip of the bladder cannula is connected through a T-shaped tube to a pressure transducer (Statham P23XL) and to a peristaltic pump (Gilson minipuls 2) for continuos infusion of a warm (37° C.) saline solution into the urinary bladder, at a constant rate of 0.1 ml/minute. The intraluminal-pressure signal during infusion of saline into the bladder is continuously recorded on a polygraph (Rectigraph-8K San-ei with BM614/2 amplifier from Biomedica Mangoni). The cystometrogram is used to evaluate the urodynamic parameters of bladder volume capacity (BVC) and micturition pressure (MP). BVC (ml) is defined as the volume of saline infused into the bladder necessary to induce detrusor contraction followed by micturition. MP (mmHg) is defined as the maximal intravesical pressure caused by contraction during micturition. Basal BVC and MP values are evaluated as mean of the values observed in the cystometrograms recorded in an initial period of 30-60 minutes. Following determination of basal BVC and MP, the infusion is interrupted and the test compounds are administered orally by a stomach tube. Bladder infusion is resumed and changes in BVC and MP are evaluated from the mean values obtained in the cystometrograms observed during 1, 2, 3, 4 and 5 hours after treatment. Compounds are administered in a volume of 2 ml/kg and groups of control animals received the same amount of vehicle (0.5% methocel in water) orally.

Statistical Analysis

Data are expressed as mean±standard error. The percent changes of BVC and MP versus the basal values, as well as Δ values (difference in ml or mmHg) of BVC and MP (BVC or MP at time “×” minus basal value), are also evaluated for each rat/time. Data are reported as % changes versus basal values.

Statistical analysis on BVC and MP values, as well as on Δ values, are performed by S.A.S./STAT software, version 6.12. The observed differences between vehicle (control) and test treatments are evaluated on Δ values of BVC and MP, whereas the differences between the values at different times versus basal values are analyzed on original BVC and MP data.

### EXAMPLE 69

### Inhibition of Stereotypy (Rhythmic Forepaw Treading) Induced by 8-OH-DPAT in Rats (Post-synaptic Antagonism)

A. Method

The inhibitory effect of 5-HT_{1A}-receptor antagonists on stereotyped forepaw treading induced in rats by subcutaneous injection of 8-OH-DPAT was evaluated by the method of Tricklebank (Tricklebank et al., Eur. J. Pharmacol., 117: 15, 1985) with minor modifications as described below.

Male Sprague-Dawley rats[Crl: CD® (SD) IGS BR] weighing 150-175 g from Charles River Italia were used. The animals were housed with free access to food and water and maintained on a forced 12-hour-light/12-hour-dark cycle at 22-24° C. of temperature. On the day of the experiment, the rats were placed singly in clear plastic containers, 10-15 minutes before administration of the vehicle or compounds to be tested. For evaluation of antagonistic activity after oral administration, the compounds were administered 1 and 4 hours before induction of stereotypy by 8-OH-DPAT (1 mg/kg subcutaneously). Observation sessions last 30 seconds and were begun 3 minutes after 8-OH-DPAT treatment and repeated every 3 minutes over a period of 15 minutes.

The appearance of the symptom induced by postsynaptic stimulation of 5-HT_{1A} receptors was noted, and the intensity was scored using an intensity scale in which: 0=absent, 1=equivocal, 2=present and 3=intense. Behavioural scores for treated rats were accumulated throughout the observation time (5 observation periods) and expressed as mean values of 4 rats/dose. Change in mean values of treated animals in comparison with control (vehicle) group, expressed as per-cent inhibition, were used to quantify the antagonistic activity.

B. Result

The results obtained are shown in table 3.

The compounds of the present invention, in particular Ex. 45, showed potent and long-lasting inhibition of stereotypy induced by 8-OH-DPAT.

TABLE 3
Inhibition of forepaw treading induced by 8-OH-DPAT in rats
(post-synaptic antagonism)

| | | % Inhibition of | |
|---|---|---|---|
| | Dose | forepaw treading | |
| Example | (mg/kg p.o.) | 1 h | 4 h |
| Ex. 16 | 10 | 40 | 17 |
| Ex. 31 | 10 | 60 | 18 |
| Ex. 45 | 10 | 100 | 81 |
| Ex. 47 | 10 | 56 | 58 |
| Ex. 48 | 10 | 90 | 74 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties.

We claim

1. A compound of the formula I: 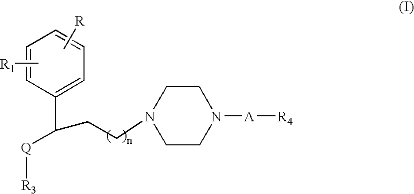
wherein
R represents hydrogen or one or more substituent selected from the group consisting of (C₁-C₆)-alkyl, (C₁-C₆)-alkoxy, (C₁-C₆)-alkylthio, hydroxy, halo, (C₂-C₆)-alkenyl, (C₂-C₆)-alkynyl, (C₁-C₆)-haloalkyl, (C₁-C₆)-haloalkoxy, (C₁-C₆)-hydroxyalkyl, alkoxyalkyl, nitro, amino, (C₁-C₆)-aminoalkyl, (C₁-C₆)-alkylamino-(C₁-C₆)-alkyl, (C₁-C₆)-alkylamino, di-(C₁-C₆)-alkylamino, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups;
R₁ represents a member selected from the group consisting of hydrogen, cycloalkyl, aryl, aryloxy, aralkyl, aralkoxy, heterocyclic, heterocycloxy, heterocycloalkyl and heterocycloalkoxy groups, each group being optionally substituted with one or more substituent R, defined as above;
Q represents —C(O)— or —CH(OR₂)— where R₂ represents a member selected from the group consisting of hydrogen, (C₁-C₆)-alkyl, (C₂-C₆)-alkenyl, (C₂-C₆)-alkynyl and cycloalkyl groups, wherein each group is optionally substituted with one or more groups selected from R₅ and R₆, where R₅ is selected from the group consisting of halo, (C₁-C₆)-alkoxy, (C₁-C₆)-haloalkoxy, cyano, (C₁-C₆)-alkoxycarbonyl, (C₁-C₆)-alkylcarbonyl, alkoxyalkyl, aminocarbonyl, N-(C₁-C₆)-alkylaminocarbonyl, N,N-di-(C₁-C₆)-alkylaminocarbonyl groups and R₆ is selected from the group consisting of aryl, heteroaryl, aryloxy, heteroaryloxy, arylalkoxy, and heteroarylalkoxy groups, each optionally substituted with R, or R₂ represents —C(O)— (C₁-C₆)-alkyl, —C(O)O—(C₁-C₆)-alkyl, —C(O)NR₇R₈ or —C(S)NR₇R₈ wherein R₇ and R₈ are independently hydrogen or (C₁-C₆)-alkyl;
R₃ represents hydrogen or a (C₁-C₆)-alkyl, (C₂-C₆)-alkenyl, (C₂-C₆)-alkynyl, cycloalkyl, aryl or heterocycle group, each group being optionally substituted with one or more substituent R or R₁, defined as above;
R₄ represents an aryl or heterocyclic group, each being optionally substituted with one or more substituent R, defined as above;
A represents a bond or (CH₂)ₙ; and
n=1 or 2,
provided that excluded are compounds wherein simultaneously Q represents —C(O)— or —CH(OR₂)— where R₂=hydrogen; R represents a hydrogen atom or one or more halogen atom, alkyl, alkoxy, haloalkyl, nitro, amino, alkylamino or di-alkylamino group; R₁ represents a hydrogen atom, unsubstituted phenyl or phenyl substituted with one or more halogen atom, alkyl or alkoxy group; R₄ is an unsubstituted aryl or unsubstituted heteroaryl group, or an aryl or heteroaryl group substituted with one or more substituent selected from the group consisting of alkyl, alkoxy, halogen, haloalkyl, nitro, amino, alkylamino, di-alkylamino, hydroxy, hydroxyalkyl, —CONR₇R₈, wherein R₇ and R₈ are independently hydrogen or alkyl, and —NHSO₂-alkyl groups; and R₃ represents an unsubstituted aryl, or unsubstituted heteroaryl, or an aryl or heteroaryl group substituted with one or more substituent selected from the group consisting of alkyl, alkoxy, halogen, haloalkyl, nitro, amino, alkylamino, di-alkylamino, phenyl, halophenyl, alkylphenyl and alkoxyphenyl groups;
provided further that also exlcuded are compounds wherein simultaneously Q represents —C(O)— or —CH(OR₂)— where R₂=hydrogen; R represents hydrogen, alkyl, alkoxy, halogen, haloalkyl, alklythio, alkenyl or alkynyl; R₁ represents hydrogen or unsubstituted cycloalkyl or cycloalkyl substituted with alkyl; R₄ represents an unsubstituted aryl or unsubstituted heteroaryl group, or an aryl or heteroaryl group substituted with one to three substituents selected from the group consisting of alkyl, alkoxy, halogen, haloalkyl, alklythio, alkenyl and alkynyl groups; and R₃ represents an unsubstituted phenyl, unsubstituted naphthyl or unsubstituted cycloalkyl group, or phenyl, naphthyl or cycloalkyl substituted with one or two substituents selected from the group consisting of alkyl, alkoxy, halo, haloalkyl, alklythio, alkenyl and alkynyl groups;
provided further that also excluded are compounds wherein simultaneously Q represents —C(O)—; R represents one more substituents selected from the group consisting of alkyl, alkoxy, alkylthio, halo, polyhaloalkyl, nitro, amino, alkylamino, alkylamino and cyano groups; R₁ represents a hydrogen atom; R₄ is a radical selected from the group consisting of indolyl, isoindolyl, quinolinyl, isoquinolinlyl, indazolyl and benzotriazolyl, or one of the foregoing radicals substituted with one or more substituent selected from the group consisting of alkyl, alkoxy, hydroxy, halo, haloalkyl, hydroxyalkyl, alkoxyalkyl, nitro, amino, alkylamino, di-alkylamino, alkoxycarbonyl, alkylcarbonyl, alkylcarbonylalkyl and alkanoyloxyalkyl groups; and R₃ represents a saturated heterocyclic ring comprising a nitrogen atom, through which said saturated heterocyclic ring is bonded to the adjacent carbonyl group at Q, and which may optionally include a further hetero atom, and which may also be optionally substituted with an alkyl group;
or an enantiomer, optical isomer, diastereomer, N-oxide, crystalline form, hydrate, solvate or pharmaceutically acceptable salt thereof.

2. The compound of claim 1 wherein
provided that, if Q represents —C(O)— or —CH(OH)— and R₃ represents a cycloalkyl, aryl or heteroaryl group, then
R represents one or more substituents selected from hydroxy, (C1-C₆)-haloalkoxy, (C1-C₆)-hydroxyalkyl, alkoxyalkyl, alkylaminoalkyl, acylamino, alkylsulphonylamino, aminosulphonyl, alkylaminosulphonyl, cyano, (C1-C₆)-aminocarbonyl, N-(C1-C₆)-alkylaminocarbonyl, N,N-di-(C1-C₆)-alkylaminocarbonyl, (C1-C₆)-alkoxycarbonyl, (C1-C₆)-alkylcarbonyl, alkylcarbonylalkyl, formyl, alkanoyloxyalkyl, alkylaminocarbonylamino, (C1-C₆)-alkylsulphinyl, (C1-C₆)-alkylsulphonyl and N,N-di-(C1-C₆)-alkylaminosulphonyl groups.

3. The compound of claim 1 wherein
R represents one or more member selected from the group consisting of hydroxy, (C₁-C₆)-haloalkoxy, (C₁-C₆)-hydroxyalkyl, alkoxyalkyl, (C₁-C₆)-aminoalkyl, (C₁-C₆)-alkylamino-(C₁-C₆)-alkyl, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups; or
R₁ represents a member selected from the group consisting of unsubstituted aryloxy, aralkyl, aralkoxy, heterocycloxy, heterocycloalkyl and heterocycloalkoxy groups, or a member selected from the group consisting of aryloxy, aralkyl, aralalkoxy, heterocycloxy, heterocycloalkyl, heterocycloalkoxy, aryl, heterocyclic and cycloalkyl groups substituted with one or more substituent selected from the group consisting of (C₁-C₆)-alkylthio, hydroxy, (C₂-C₆)-alkenyl, (C₂-C₆)-alkynyl, (C₁-C₆)-haloalkoxy, (C₁-C₆)-hydroxyalkyl, alkoxyalkyl, (C₁-C₆)-aminoalkyl, (C₁-C₆)-alkylamino-(C₁-C₆)-alkyl, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups.

4. The compound of claim 1 wherein R₃ represents a hydrogen atom or a (C₁-C₆)-alkyl, (C₂-C₆)-alkenyl, (C₂-C₆)-alkynyl group, each group being optionally substituted with one or more substituent selected from the group consisting of R and R₁.

5. The compound of claim 1 wherein R₄ represents a substituted aryl or substituted heterocyclic group, each group substituted with one or more substituent selected from the group consisting of (C₁-C₆)-haloalkoxy, alkoxyalkyl, (C₁-C₆)-aminoalkyl, (C₁-C₆)-alkylamino-(C₁-C₆)-alkyl, acylamino, aminosulphonyl, (C₁-C₆)-alkylaminosulphonyl, cyano, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups.

6. The compound of formula I wherein
R represents a hydrogen or halogen atom or (C₁-C₆)-alkoxy, (C₁-C₆)-haloalkoxy, N,N-di-(C₁-C₆)-aminocarbonyl or cyano group;
R₁ represents is a hydrogen atom;
Q represents —C(O)— or —CH(OR₂)— where R₂ represents a hydrogen atom or (C₁-C₆)-alkyl, (C₂-C₆)-alkenyl, (C₂-C₆)-alkynyl, —C(O)— (C₁-C₆)-alkyl, —C(O)O—(C₁-C₆)-alkyl, —C(O)NR₇R₈ or —C(S)NR₇R₈ wherein R₇ and R₈ are independently hydrogen or (C₁-C₆)-alkyl group;
R₃ represents a hydrogen atom or a (C₁-C₆)-alkyl, (C₂-C₆)-alkenyl, (C₂-C₆)-alkynyl, cycloalkyl, aryl or heterocyclic group;
R₄ represents are an aryl or heterocyclic group, each being optionally substituted with one or more substituent selected from the group consisting of halogen atom, (C₁-C₆)-alkoxy and (C₁-C₆)-haloalkoxy groups;
A represents a bond; and
n=1.

7. The compound of claim 1 wherein said compound is a member selected from the group consisting of
1-[4-cyclohexyl-3-(2-fluorophenyl)-4-methoxybutyl]-4-[2-(2,2,2-trifluoroethoxy)phenyl]piperazine;
1-(4-Fluoro-2-methoxyphenyl)-4-[4-oxo-3-(2-trifluoromethoxyphenyl)pentyl]piperazine;
1-(4-Fluoro-2-methoxyphenyl)-4-[4-hydroxy-3-(2-trifluoromethoxyphenyl)pentyl]piperazine;
1-[5-(2,3-Dihydro-1,4-benzodioxinyl)]-4-[4-oxo-3-(2-trifluoromethoxyphenyl)pentyl]piperazine;
1-[5-(2,3-Dihydro-1,4-benzodioxinyl)]-4-[4-hydroxy-3-(2-trifluoromethoxyphenyl)pentyl]piperazine;
1-[5-(2,3-Dihydro-1,4-benzodioxinyl)]-4-[4-hydroxy-3-(2-trifluoromethoxyphenyl)hexyl]piperazine;
1-[5-(2,3-Dihydro-1,4-benzodioxinyl)]-4-[4-hydroxy-3-(2-trifluoromethoxyphenyl)hex-5-enyl]piperazine;
1-[5-(2,3-Dihydro-1,4-benzodioxinyl)]-4-[4-hydroxy-5-methyl-3-(2-trifluoromethoxyphenyl)hexyl]piperazine;
1-[5-(2,3-Dihydro-1,4-benzodioxinyl)]-4-[4-methoxy-3-(2-trifluoromethoxyphenyl)-5-hexenyl]piperazine;
1-[5-(2,3-Dihydro-1,4-benzodioxinyl)]-4-[(4-methoxy-3-phenyl)heptyl]piperazine;
1-[5-(2,3-Dihydro-1,4-benzodioxinyl)]-4-[(4-methoxy-3-phenyl)pentyl]piperazine;
1-[5-(2,3-Dihydro-1,4-benzodioxinyl)]-4-[(4-propoxy-3-phenyl)heptyl]piperazine;
1-[3-(2-Cyanophenyl)-4-cyclohexyl-4-oxo-butyl]-4-[5-(2,3-dihydro-1,4-benzodioxinyl)]piperazine;
1-[3-(2-Cyanophenyl)-4-cyclohexyl-4-hydroxybutyl]-4-[5-(2,3-dihydro-1,4-benzodioxinyl)]piperazine;
1-[3-(2-Cyanophenyl)-4-cyclohexyl-4-hydroxybutyl]-4-[5-(2,3-dihydro-1,4-benzodioxinyl)]piperazine;
1-[3-(2-Cyanophenyl)-4-cyclohexyl-4-hydroxybutyl]-4-[5-(2,3-dihydro-1,4-benzodioxinyl)]piperazine;
1-[3-(2-Cyanophenyl)-4-cyclohexyl-4-oxobutyl]-4-(4-fluoro-2-methoxyphenyl)piperazine;
1-[3-(2-Cyanophenyl)-4-cyclohexyl-4-hydroxybutyl]-4-(4-fluoro-2-methoxyphenyl)piperazine;
1-(4-cyclohexyl-4-methoxy-3-phenylbutyl)-4-[5-(2,3-dihydro-1,4-benzodioxinyl)]piperazine;
1-(4-Cyclohexyl-4-methoxy-3-phenylbutyl)-4-(4-fluoro-2-methoxyphenyl)piperazine;
1-(4-Cyclohexyl-4-ethoxy-3-phenylbutyl)-4-[5-(2,3-dihydro-1,4-benzodioxinyl)]piperazine;
1-(4-Cyclohexyl-4-ethoxy-3-phenylbutyl)-4-(4-fluoro-2-methoxyphenyl)piperazine;
1-(4-Allyloxy-4-cyclohexyl-3-phenylbutyl)-4-[5-(2,3-dihydro-1,4-benzodioxinyl)]piperazine;
1-(4-Allyloxy-4-cyclohexyl-3-phenylbutyl)-4-(4-fluoro-2-methoxyphenyl)piperazine;
1-(4-Cyclohexyl-3-phenyl-4-propargyloxybutyl)-4-[5-(2,3-dihydro-1,4-benzodioxinyl)]piperazine;
1-(4-Cyclohexyl-3-phenyl-4-propargyloxybutyl)-4-(4-fluoro-2-methoxyphenyl)piperazine;
1-(4-Cyclohexyl-3-phenyl-4-propoxybutyl)-4-[5-(2,3-dihydro-1,4-benzodioxinyl)]piperazine;
1-[5-(2,3-Dihydro-1,4-benzodioxinyl)]-4-(4-hydroxy-3-phenyl)hexylpiperazine;
1-[5-(2,3-Dihydro-1,4-benzodioxinyl)]-4-(4-hydroxy-3-phenyl)heptyl]piperazine;
1-[5-(2,3-Dihydro-1,4-benzodioxinyl)]-4-(4-hydroxy-3-phenylhex-5-enyl]piperazine;
1-[5-(2,3-Dihydro-1,4-benzodioxinyl)]-4-(4-hydroxy-5-methyl-3-phenyl)hexyl]piperazine;
1-[5-(2,3-Dihydro-1,4-benzodioxinyl)]-4-(4-hydroxy-3-phenyl)pentyl]piperazine;
1-[5-(2,3-Dihydro-1,4-benzodioxinyl)]-4-(4-hydroxy-3-phenylhept-5-ynyl)piperazine;
1-[5-(2,3-Dihydro-1,4-benzodioxinyl)]-4-(4-hydroxy-3-phenylhept-5-enyl)piperazine;
1-[5-(2,3-Dihydro-1,4-benzodioxinyl)]-4-(4-hydroxy-3-phenylhex-5-ynyl)piperazine;
1-[5-(2,3-Dihydro-1,4-benzodioxinyl)]-4-(4-hydroxy-3-phenylhept-6-enyl)piperazine;
1-[5-(2,3-Dihydro-1,4-benzodioxinyl)]-4-(4-hydroxy-6-methyl-3-phenylhept-5-enyl)piperazine;
1-[5-(2,3-Dihydro-1,4-benzodioxinyl)]-4-(4-hydroxy-6-methyl-3-phenyl)heptyl]piperazine;
1-[5-(2,3-dihydro-1,4-benzodioxinyl)]-4-(4-hydroxy-3-phenylbutyl]piperazine;
1-[5-(2,3-Dihydro-1,4-benzodioxinyl)]-4-(4-hydroxy-3-phenylpentyl)piperazine;
1-[4-Cyclohexyl-3-(2-dimethylaminocarbonylphenyl)-4-oxobutyl]-4-[5-(2,3-dihydro-1,4-benzodioxinyl)]piperazine;
1-[4-Cyclohexyl-3-(2-dimethylaminocarbonylphenyl)-4-hydroxybutyl]-4-[5-(2,3-dihydro-1,4-benzodioxinyl)]piperazine;
1-[4-Cyclohexyl-3-(2-dimethylaminocarbonylphenyl)-4-oxobutyl]-4-(4-fluoro-2-methoxyphenyl)piperazine;
1-[4-Cyclohexyl-3-(2-dimethylaminocarbonylphenyl)-4-hydroxybutyl]-4-(4-fluoro-2-methoxyphenyl)piperazine;
1-[3-(2-Cyanophenyl)-4-oxopentyl]-4-[5-(2,3-dihydro-1,4-benzodioxinyl)]piperazine;
1-[4-Cyclohexyl-3-(2-trifluoromethoxyphenyl)-4-oxobutyl]-4-(4-indolyl)piperazine;
1-[4-Acetoxy-4-cyclohexyl-3-(2-fluorophenyl)butyl]-4-(2-methoxyphenyl)piperazine;
1-[4-Cyclohexyl-3-(2-fluorophenyl)-4-methoxycarbonyloxybutyl]-4-(2-methoxyphenyl)piperazine;
1-[4-Cyclohexyl-4-ethylaminocarbonyloxy-3-(2-fluorophenyl)butyl]-4-(2-methoxyphenyl)piperazine;
1-[4-Aminocarbonyloxy-4-cyclohexyl-3-(2-fluorophenyl)butyl]-4-(2-methoxyphenyl)piperazine;
1-[5-(2,3-Dihydro-1,4-benzodioxinyl)]-4-(4-hydroxy-5,5-dimethyl-3-phenyl)hexyl]piperazine;
1-(4-Fluoro-2-methoxyphenyl)-4-[(4-hydroxy-3-phenyl)hept-5-ynyl]piperazine;
1-(4-Fluoro-2-methoxyphenyl)-4-[(4-hydroxy-3-phenyl)hept-5-enyl]piperazine;
1-(4-Fluoro-2-methoxyphenyl)-4-[(4-hydroxy-3-phenyl)hept-5-enyl]piperazine;
1-[5-(2,3-Dihydro-1,4-benzodioxinyl)]-4-[(4-hydroxy-5-methyl-3-phenyl)hex-5-enyl]piperazine;
1-[5-(2,3-Dihydro-1,4-benzodioxinyl)]-4-[(4-hydroxy-6-methyl-3-phenyl)hept-6-enyl]piperazine;
1-[5-(2,3-Dihydro-1,4-benzodioxinyl)]-4-[[4-hydroxy-4-(2-thienyl)-3-phenyl]butyl]piperazine;
1-[5-(2,3-Dihydro-1,4-benzodioxinyl)]-4-[(4-hydroxy-3-phenyl)octyl]piperazine;
1-(4-Fluoro-2-methoxyphenyl)-4-[(4-methoxy-3-phenyl)hept-5-ynyl]piperazine;
1-(4-Fluoro-2-methoxyphenyl)-4-[(4-methoxy-3-phenyl)hept-5-enyl]piperazine;
1-[4-Cyclohexyl-3-(2-methoxymethylphenyl)-4-oxobutyl]-4-(4-fluoro-2-methoxyphenyl)piperazine;
1-[4-Cyclohexyl-4-hydroxy-3-(2-methoxymethylphenyl)-butyl]-4-(4-fluoro-2-methoxyphenyl)piperazine;
1-[5-(2,3-Dihydro-1,4-benzodioxinyl)]-4-[4-cyclohexyl-3-(2-methoxymethylphenyl)-4-oxobutyl]-piperazine;
1-[4-Cyclohexyl-4-hydroxy-3-(2-methoxymethylphenyl)-butyl]-4-(2,3-dihydro-1,4-benzodioxinyl)piperazine; and
1-[4-Cyclohexyl-4-methylaminothiocarbonyloxy-3-(2-fluorophenyl)butyl]-4-(2-methoxyphenyl)piperazine.

8. The compound according to claim 7 that is a member selected from the group consisting of
(RS,SR)-1-[3-(2-Cyanophenyl)-4-cyclohexyl-4-hydroxybutyl]-4-[5-(2,3-dihydro-1,4-benzodioxinyl)]piperazine;
(RS)-1-[3-(2-Cyanophenyl)-4-cyclohexyl-4-hydroxybutyl]-4-[5-(2,3-dihydro-1,4-benzodioxinyl)]piperazine;
(SR)-1-[3-(2-Cyanophenyl)-4-cyclohexyl-4-hydroxybutyl]-4-[5-(2,3-dihydro-1,4-benzodioxinyl)]piperazine;
(RS,SR)-1-[5-(2,3-Dihydro-1,4-benzodioxinyl)]-4-(4-hydroxy-3-phenylpentyl)piperazine;
(RS,SR) 1-[4-Acetoxy-4-cyclohexyl-3-(2-fluorophenyl)butyl]-4-(2-methoxyphenyl)piperazine;
(RS,SR) 1-[4-Cyclohexyl-3-(2-fluorophenyl)-4-methoxycarbonyloxybutyl]-4-(2-methoxyphenyl)piperazine;
(RS,SR) 1-[4-Cyclohexyl-4-ethylaminocarbonyloxy-3-(2-fluorophenyl)butyl]-4-(2-methoxyphenyl)piperazine;
(RS,SR) 1-[4-Aminocarbonyloxy-4-cyclohexyl-3-(2-fluorophenyl)butyl]-4-(2-methoxyphenyl)piperazine;
(E,Z)-1-(4-Fluoro-2-methoxyphenyl)-4-[(4-hydroxy-3-phenyl)hept-5-enyl]piperazine;
(E)-1-(4-Fluoro-2-methoxyphenyl)-4-[(4-hydroxy-3-phenyl)hept-5-enyl]piperazine; and
(RS,SR) 1-[4-Cyclohexyl-4-methylaminothiocarbonyloxy-3-(2-fluorophenyl)butyl]-4-(2-methoxyphenyl)piperazine.

9. A pharmaceutical composition comprising a compound of claim 1 or claim 7 and a pharmaceutically acceptable diluent, excipient or carrier.

10. The pharmaceutical composition of claim 9 which comprises at least one excipient selected from the group consisting of lubricants, plasticizers, colorants, absorption enhancers, and bactericides.

11. A method of treating neuromuscular dysfunction of the lower urinary tract in a mammal in need of such treatment, comprising administering an effective amount of a compound according to claim 1 to said mammal in need of such treatment.

12. The method of 11 wherein said mammal is a human.

13. The method of claim 12 wherein administration of said compound ameliorates a condition or symptom selected from the group consisting of urinary urgency, overactive bladder, increased urinary frequency, incontinence, mixed incontinence, urine leakage, enuresis, dysuria, urinary hesitancy and difficulty in emptying the urinary bladder.

14. The method of claim 12 wherein said compound is administered via a route selected from the group consisting of oral, enteral, intravenous, intramuscular, subcutaneous, transmucosal, transdermal and by-inhalation routes.

15. The method of claim 12, wherein said compound is administered in an amount of between about 0.01 and 25 mg/kg/day.

16. The method of claim 15 wherein said compound is administered in an amount of between about 0.1 and about 10 mg/kg/day.

17. The method of claim 16, wherein said compound is administered in an amount of between about 0.2 and about 5 mg/kg/day.

18. The method of claim 12, wherein said compound is administered in an amount of between about 50 and 400 mg/day.

19. The method of claim 18 wherein said compound is administered via an oral or transdermal route.

20. A method of reducing the frequency of urinary bladder contractions in a mammal in need of such treatment comprising administering an effective amount of a compound according to claim 1 to said mammal in need of such treatment.

21. The method of claim 20 wherein said mammal is a human.

22. The method of claim 11 or 21 further comprising administering said compound in combination with an antimuscarinic or α₁ antagonist.

23. The method of claim 22 wherein said antimuscarinic is selected from the group consisting of oxybutynin, tolterodine, darifenacin, and temiverine.

24. The method of claim 22 wherein said α₁ antagonist is selected from the group consisting of prazosin, doxazosin, terazosin, alfuzosin, and tamsulosin.

25. A method of treating neuromuscular dysfunction of the lower urinary tract in a mammal in need of such treatment, comprising administering an effective amount of a compound according to claim 7 to said mammal in need of such treatment.

26. The method of 25 wherein said mammal is a human.

27. A method of reducing the frequency of urinary bladder contractions in a mammal in need of such treatment comprising administering an effective amount of a compound according to claim 7 to said mammal in need of such treatment.

28. The method of claim 27 wherein said mammal is a human.

29. A method for treating disorders of the central nervous system caused by serotonergic dysfunction, comprising delivering an effective amount of a compound according to claim 1 or claim 7 to the environment of a 5-HT_{1A} serotonergic receptor.

30. The method of claim 29 wherein said compound is delivered via an extracorporeal route.

31. The method of claim 29 wherein said compound is delivered by administering the compound to a mammal possessing the 5-HT_{1A} serotonergic receptor.

32. A method for treating diseases associated with activity of a 5-HT_{1A} serotonergic receptor, which diseases are treated by reducing said activity, the method comprising exposing said receptor to an effective amount of a compound according to claim 1 or claim 7, thereby blocking said receptor and lowering the activity of said receptor.

33. A method of antagonizing the serotonin HT_{1A} receptor comprising administering to a patient in need of such treatment an effective amount of a compound of claim 1 or claim 7, thereby antagonizing said receptor.

34. A pharmaceutical composition suitable for administration to a mammal which comprises a compound of claim 1 or claim 7, wherein said compound has an enantiomeric enrichment (ee) of greater than 0%.

35. The composition of claim 34 which comprises a predetermined amount of at least one enantiomer of said compound.

36. A method for treating a disorder of the central nervous system caused by serotonergic dysfunction, comprising delivering an effective amount of a composition according to claim 34 to the environment of a 5-HT_{1A} serotonergic receptor.

37. The method of claim 36 wherein said disorder is selected from the group consisting of anxiety, depression, hypertension, sleep/wake cycle disorders, feeding disorders, behaviour disorders, sexual dysfunction and cognition disorders associated with stroke, injury, dementia, and originated by neurological development, attention-deficit hyperactivity disorders (ADHD), drug addiction, drug withdrawal, irritable-bowel syndrome and symptoms caused by withdrawal or partial withdrawal from the use of nicotine or tobacco.

38. The method of claim 36 wherein said composition is delivered via an extracorporeal route.

39. The method of claim 38 wherein said composition is delivered by administering the compound to a mammal possessing a 5-HT_{1A} serotonergic receptor.

40. A compound represented by the formula 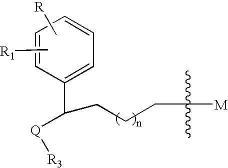
wherein
M represents the group 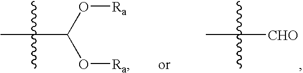
R represents hydrogen or one or more substituents selected from the group consisting of (C₁-C₆)-alkyl, (C₁-C₆)-alkoxy, (C₁-C₆)-alkylthio, hydroxy, halo, (C₂-C₆)-alkenyl, (C₂-C₆)-alkynyl, (C₁-C₆)-haloalkyl, (C₁-C₆)-haloalkoxy, (C₁-C₆)-hydroxyalkyl, alkoxyalkyl, nitro, amino, (C₁-C₆)-aminoalkyl, (C₁-C₆)-alkylamino-(C₁-C₆)-alkyl, (C₁-C₆)-alkylamino, di-(C₁-C₆)-alkylamino, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups;
R₁ represents a member selected from the group consisting of hydrogen, cycloalkyl, aryl, aryloxy, aralkyl, aralkoxy, heterocyclic, heterocycloxy, heterocycloalkyl and heterocycloalkoxy groups, each group being optionally substituted with one or more substituent R, defined as above;
Q represents —C(O)— or —CH(OR₂)— where R₂ represents a member selected from the group consisting of hydrogen, (C₁-C₆)-alkyl, (C₂-C₆)-alkenyl, (C₂-C₆)-alkynyl and cycloalkyl groups, wherein each group is optionally substituted with one or more groups selected from R₅ and R₆, where R₅ is selected from the group consisting of halo, (C₁-C₆)-alkoxy, (C₁-C₆)-haloalkoxy, cyano, (C₁-C₆)-alkoxycarbonyl, (C₁-C₆)-alkylcarbonyl, alkoxyalkyl, aminocarbonyl, N-(C₁-C₆)-alkylaminocarbonyl, N,N-di-(C₁-C₆)-alkylaminocarbonyl groups and R₆ is selected from the group consisting of aryl, heteroaryl, aryloxy, heteroaryloxy, arylalkoxy, and heteroarylalkoxy groups, each optionally substituted with R, or R₂ represents —C(O)—(C₁-C₆)-alkyl, —C(O)O—(C₁-C₆)-alkyl, —C(O)NR₇R₈ or —C(S)NR₇R₈ wherein R₇ and R₈ are independently hydrogen or (C₁-C₆)-alkyl;
R₃ represents hydrogen or a (C₁-C₆)-alkyl, (C₂-C₆)-alkenyl, (C₂-C₆)-alkynyl, cycloalkyl, aryl or heterocycle group, each group being optionally substituted with one or more substituent R or R₁, defined as above;
Rₐ represent (C₁-C₆)-alky groups that may be the same or different, or together
form an alkylene chain of 3 to 5 carbon; and
n=0 or 1,
provided that excluded are compounds wherein simultaneously Q represents —C(O)—; M represents —CHO; R represents hydrogen, alkyl, alkoxy, halogen, haloalkyl, alklythio, alkenyl or alkynyl; R₁ represents hydrogen or unsubstituted cycloalkyl or cycloalkyl substituted with alkyl; and R₃ represents an unsubstituted phenyl, unsubstituted naphthyl or unsubstituted cycloalkyl group, or phenyl, naphthyl or cycloalkyl substituted with one or two substituents selected from the group consisting of alkyl, alkoxy, halo, haloalkyl, alklythio, alkenyl and alkynyl groups

41. A compound represented by the formula 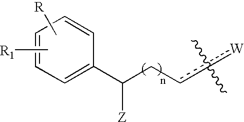
wherein
W represents the group 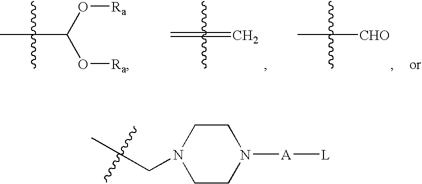
R represents hydrogen or one or more substituents selected from the group consisting of (C₁-C₆)-alkyl, (C₁-C₆)-alkoxy, (C₁-C₆)-alkylthio, hydroxy, halo, (C₂-C₆)-alkenyl, (C₂-C₆)-alkynyl, (C₁-C₆)-haloalkyl, (C₁-C₆)-haloalkoxy, (C₁-C₆)-hydroxyalkyl, alkoxyalkyl, nitro, amino, (C₁-C₆)-aminoalkyl, (C₁-C₆)-alkylamino-(C₁-C₆)-alkyl, (C₁-C₆)-alkylamino, di-(C₁-C₆)-alkylamino, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups;
R₁ represents a member selected from the group consisting of hydrogen, cycloalkyl, aryl, aryloxy, aralkyl, aralkoxy, heterocyclic, heterocycloxy, heterocycloalkyl and heterocycloalkoxy groups, each group being optionally substituted with one or more substituent R, defined as above;
Z represents a —CHO, cyano, or —CH(ORₐ)₂ group, [symbol]
represents a single or double bond,
Rₐ represent (C₁-C₆)-alky groups that may be the same or different, or together form an alkylene chain of 3 to 5 carbon; and
L represents an aryl or heterocyclic group, each being optionally substituted with one or more substituent R, defined as above;
A represents a bond or (CH₂)ₙ; and
n=0 or 1.
